(19) United States
(12) Patent Application Publication
(10) Pub. No.: US 2014/0004510 A1
(43) Pub. Date: Jan. 2, 2014

(54) METHODS AND COMPOSITIONS FOR PROGNOSING AND/OR DETECTING AGE-RELATED MACULAR DEGENERATION

(71) Applicants: Margaret M. DeAngelis, Bountiful, UT (US); Margaux Morrison, Boston, MA (US)

(72) Inventors: Margaret M. DeAngelis, Bountiful, UT (US); Margaux Morrison, Boston, MA (US)

(73) Assignee: MASSACHUSETTS EYE AND EAR INFIRMARY, Boston, MA (US)

(21) Appl. No.: 13/825,855

(22) PCT Filed: Sep. 23, 2011
(86) PCT No.: PCT/US2011/053069

§ 371 Date: Sep. 17, 2013

Related U.S. Application Data
(60) Provisional application No. 61/386,445, filed on Sep. 24, 2010.

(51) Int. Cl. C12Q 1/68

(52) U.S. Cl. CPC C12Q 1/6883
USPC 435/6.11

(57) ABSTRACT

The invention is based, in part, upon the discovery of single nucleotide polymorphisms (SNPs) and haplotypes located in promoter and intronic sequences (e.g., intron 2) of the roundabout, axon guidance receptor, homolog 1 (ROBO1) gene that are significantly associated with age-related macular degeneration (AMD) risk. The invention relates to methods and compositions for determining whether an individual is at risk of developing age-related macular degeneration by detecting whether the individual has a protective or risk variant of the ROBO1 gene.

### CROSS REFERENCE TO RELATED APPLICATIONS

This application claims the benefit of and priority to U.S. Provisional Patent Application No. 61/386,445, filed Sep. 24, 2010, the content of which is hereby incorporated by reference in its entirety.

### GOVERNMENT FUNDING

The work described in this application was sponsored, in part, by the National Eye Institute under Grant No. EY014458 and EY14104. The United States Government has certain rights in the invention.

### FIELD OF THE INVENTION

The methods and compositions disclosed herein relate to determining whether an individual is at risk of developing age-related macular degeneration by detecting whether the individual has a protective or risk variant of the ROBO1 gene.

### BACKGROUND

There are a variety of chronic intraocular disorders, which, if untreated, may lead to partial or even complete vision loss. One prominent chronic intraocular disorder is age-related macular degeneration, which is the leading cause of blindness amongst elderly Americans affecting a third of patients aged 75 years and older (Fine et al. (2000) N. ENGL. J. MED. 342: 483-492). There are two forms of age-related macular degeneration (“AMD”), a dry form and a wet (also known as a neovascular) form.

The dry form involves a gradual degeneration of a specialized tissue beneath the retina, called the retinal pigment epithelium, accompanied by the loss of the overlying photoreceptor cells. These changes result in a gradual loss of vision. The wet form is characterized by the growth of new blood vessels beneath the retina which can bleed and leak fluid, resulting in a rapid, severe and irreversible loss of central vision in the majority cases. This loss of central vision adversely affects one's everyday life by impairing the ability to read, drive and recognize faces. In some cases, the macular degeneration progresses from the dry form to the wet form, and there are at least 200,000 newly diagnosed cases a year of the wet form (Hawkins et al. (1999) MOL. VISION 5: 26-29). The wet form accounts for approximately 90% of the severe vision loss associated with age-related macular degeneration.

At this time, current diagnostic methods cannot accurately predict the risk of age-related macular degeneration for an individual. Unfortunately, the degeneration of the retina has already begun by the time age-related macular degeneration is diagnosed in the clinic. Further, most current treatments are limited in their applicability, and are unable to prevent or reverse the loss of vision especially in the case of the wet type, the more severe form of the disease (Miller et al. (1999) ARCH. OPHTHALMOL. 117(9): 1161-1173).

Currently, the treatment of the dry form of age-related macular degeneration includes administration of antioxidant vitamins and/or zinc. Treatment of the wet form of age-related macular degeneration, however, has proved to be more difficult.

Several methods have been approved in the United States of America for treating the wet form of age-related macular degeneration. Two are laser based approaches, and include laser photocoagulation and photodynamic therapy using a benzoporphyrin derivative photosensitizer known as Visudyne®. Two require the administration of therapeutic molecules that bind and inactivate or reduce the activity of Vascular Endothelial Growth Factor (VEGF), one is known as Lucentis® (ranibizumab), which is a humanized anti-VEGF antibody fragment, and the other is known as Macugen (pegaptanib sodium injection), which is an anti-VEGF aptamer.

During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss.

During photodynamic therapy, a benzoporphyrin derivative photosensitizer known as Visudyne® and available from QLT, Inc. (Vancouver, Canada) is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510).

Lucentis®, which is available from Genentech, Inc., CA, is a humanized therapeutic antibody that binds and inhibits or reduces the activity of VEGF, a protein believed to play a role in angiogenesis. Pegaptanib sodium, which is available from OSI Pharmaceuticals, Inc., NY, is a pegylated aptamer that targets VEGF165, the isoform believed to be responsible for primary pathological ocular neovascularization.

The variants and haplotypes most consistently associated with AMD are within the gene complement factor H (CFH) (1q32) and the locus containing the genes age-related maculopathy susceptibility 2 and HtrA serine peptidase 1 (ARMS2 and HTRA1) (10q26) (DeAngelis, et al. (2008) OPHTHALMOL, 115, 1209-1215; Dewan, et al. (2006) SCIENCE, 314, 989-992; Edwards, et al. (2005) SCIENCE, 308, 421-424; Hageman, et al. (2005) PROC. NATL. ACAD. SCI. USA, 102, 7227-7232; Haines, et al. (2005) SCIENCE, 308, 419-421; Jakobsdottir, et al. (2005) AM. J. HUM. GENET., 77, 389-407; Kanda, et al. (2007) PROC. NATL. ACAD. SCI. USA, 104, 16227-16232; Klein, et al. (2005) SCIENCE, 308, 385-389; Li, et al. (2006) NAT. GENET., 38, 1049-1054; Rivera, et al. (2005) HUM. MOL. GENET., 14, 3227-3236; Yang, et al. (2006) SCIENCE, 314, 992-993). These genes have been shown to have large influences on AMD risk in populations of various ethnicities, with variants on 10q26 being the most strongly associated with the neovascular AMD subtype (Fisher, et al. (2005) HUM. MOL. GENET., 14, 2257-2264; Shuler, et al. (2007) ARCH. OPHTHALMOL., 125, 63-67; Zhang, et al. (2008) BMC MED. GENET., 9, 51). Despite their large influence on AMD risk, the combination of these genes alone is insufficient to correctly predict the development and progression of this disease (Jakobsdottir, et al. (2009) PLoS GENET., 5, e1000337).

Therefore, there is still an ongoing need for methods of identifying individuals at risk of developing age-related macular degeneration so that such individuals can be monitored more closely and then treated to slow, stop or reverse the onset of age-related macular degeneration.

### SUMMARY

The methods and compositions disclosed herein are based, in part, upon the discovery of single nucleotide polymorphisms (SNPs) and haplotypes located in promoter and intronic sequences (e.g., intron 2) of the roundabout, axon guidance receptor, homolog 1 (ROBO1) gene that are significantly associated with age-related macular degeneration (AMD) risk. Variants at several polymorphic sites have been found to be associated with a risk of developing AMD as determined by statistical analysis, by virtue of haplotype analysis, and/or by the virtue of the fact that they cluster with variants at polymorphic sites identified by statistical or haplotype analysis. In addition, one haplotype block has been found to be associated with reduced risk of developing AMD.

Accordingly, in one aspect, disclosed herein is a method of determining a subject's, for example, a human subject's, risk of developing age-related macular degeneration. The method comprises detecting in a sample from a subject the presence or absence of an allelic variant at a polymorphic site of the ROBO1 gene that is associated with risk of developing AMD, such as a protective variant or a risk variant. If the subject has at least one protective variant, the subject is less likely to develop age-related macular degeneration than a person without the protective variant. If the subject has at least one risk variant, the subject is more likely to develop age-related macular degeneration than a person without the risk variant.

In one embodiment, a protective variant T>G (rs7615149) in the ROBO1 gene was identified that is associated with reduced risk of developing AMD (dry and/or neovascular forms of the disease).

In another embodiment, a protective variant C>T (rs59931439) in the ROBO1 gene was identified as associated with reduced risk of developing AMD (dry and/or neovascular forms of the disease).

In another embodiment, a risk variant T>C (rs9309833) in the ROBO1 gene was identified as associated with increased risk of developing AMD (dry and/or neovascular forms of the disease). However, when present in combination with variant G>A (rs8034864) of the RORA gene, risk variant T>C (rs9309833) in the ROBO1 gene was associated with decreased risk of developing AMD (dry and/or neovascular forms of the disease).

In another embodiment, a variant G>A (rs4513416) in the ROBO1 gene was identified as associated with risk of developing dry AMD. When present in combination with variant G>A (rs8034864) of the RORA gene, variant G>A (rs4513416) in the ROBO1 gene was associated with increased risk of developing dry AMD.

In another embodiment, a risk variant C>T (rs1387665) in the ROBO1 gene was identified as associated with increased risk of developing wet AMD. When present in combination with variant G>A (rs8034864) of the RORA gene, variant C>T (rs1387665) in the ROBO1 gene was associated with decreased risk of developing dry AMD.

In each of the foregoing embodiments, the common allele in the ROBO1 gene or in the RORA gene is denoted using the forward strand of the ROBO1 gene indicated in the Ensembl database.

In another aspect, the methods disclosed herein provide for determining a subject's, for example, a human subject's, risk of developing age-related macular degeneration by detecting in a sample from a subject the presence or absence of a haplotype in the ROBO1 gene (or in a region of the ROBO1 gene). If the subject has a protective haplotype, the subject is less likely to develop age-related macular degeneration than a person without the protective haplotype. If the subject has a risk haplotype, the subject is more likely to develop age-related macular degeneration than a person without the risk haplotype.

In one embodiment, a haplotype is defined by the alleles present at the polymorphic sites rs6548621 and rs7615149. The method comprises detecting a cytosine base or a thymine base at rs6548621 and a guanine base or thymine base at rs7615149. When the haplotype comprises a guanine in the forward sequence of rs7615149 and a thymine in the forward sequence of rs6548621 (e.g., in the Sibling Cohort) or a cytosine in the forward sequence of rs6548621 (e.g., in the Greek Cohort), the haplotype is a protective haplotype indicating that the subject is less likely to develop AMD than a person without this haplotype.

A variant sequence and/or a haplotype can be detected by standard techniques known in the art, which can include, for example, direct nucleotide sequencing, hybridization assays using a probe that anneals to the protective variant, to the risk variant, or to the common allele at the polymorphic site, restriction fragment length polymorphism assays, or amplification-based assays. Furthermore, it is contemplated that the polymorphic sites may be amplified prior to the detection steps. In certain embodiments, the detecting step can include an amplification reaction using primers capable of amplifying the polymorphic site.

In another aspect, disclosed herein is a method of assisting in diagnosing or assessing the risk of developing age-related macular degeneration. The method can include communicating a report indicating the presence or absence of at least one protective variant and/or the presence or absence of at least one risk variant at a polymorphic site of the ROBO1 gene in a sample from a subject, for example a human subject. The polymorphic site can include ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809. If the subject has at least one protective variant, the subject is less likely to develop age-related macular degeneration than a person without the protective variant. If the subject has at least one risk variant, the subject is more likely to develop AMD than a person without the risk variant. Alternatively, a variant (e.g., a protective variant or a risk variant), may be detected by a proxy or surrogate SNP that is in linkage disequilibrium with the protective variant.

In another aspect, disclosed herein is a method of assisting in diagnosing or assessing the risk of developing age-related macular degeneration. The method can include detecting in a sample from a subject the presence or absence of a haplotype in a region of the ROBO1 gene. If the subject has a risk haplotype, the subject is more likely to develop AMD than a person without the risk haplotype. If the subject has a protective haplotype, the subject is less likely to develop AMD than a person without the protective haplotype. A haplotype may be defined by polymorphic sites rs6548621 and rs7615149. Alternatively, a haplotype may be detected by a proxy or surrogate SNP that is in linkage disequilibrium with the haplotype, for example, a haplotype described herein.

In some embodiments, a protective variant and/or a risk variant of the ROBO1 gene, and/or a protective haplotype and/or a risk haplotype of the ROBO1 gene may be detected in combination with a protective variant and/or a risk variant at one or more of the following polymorphic sites: rs1061170 (CFH), rs800292 (CFH), rs10490924 (LOC387715), rs11200638 (ARMS2/HTRA1), rs2672598 (ARMS2/HTRA1), rs10664316 (ARMS2/HTRA1), rs1049331 (ARMS2/HTRA1), rs12900948 (RORA), rs4335725 (RORA), rs8034864 (RORA), and rs1045216 (PLEKHA1).

In another aspect, disclosed herein is a method of determining whether a subject is at risk of developing, or has, age-related macular degeneration, the method comprising measuring the amount of a ROBO1 gene product in a test sample obtained from the subject, wherein an amount of the ROBO1 gene product in the sample less than a control value is indicative that the subject is at risk of developing, or has, age-related macular degeneration. The method may further comprise measuring the amount of a RORA gene product in a test sample obtained from the subject, wherein an amount of the RORA gene product in the sample less that a control value is indicative that the subject is at risk of developing, or has, age-related macular degeneration.

In some embodiments, the method may further comprise measuring the amount of a gene product selected from the group consisting of a IGHM, NLRP2, PKP2, PLA2G4A, TANC1, and UCHL1 gene product, wherein an amount of the gene product in the sample less than a control value is indicative that the subject is at risk of developing, or has developed, age-related macular degeneration. Either additional or alternatively the method may further comprise measuring the amount of a gene product selected from the group consisting of a CREB5, CXCL13, ENPP2, FAM169A, IGKV1-5, IL1A, MMP7, PGS13, PRS6KA2, and UGT2B 17 gene product, wherein an amount of the gene product in the sample greater than a control value is indicative that the subject is at risk of developing, or has developed, age-related macular degeneration.

The test sample may be a tissue or body fluid sample. Exemplary body fluid samples include blood, serum, and plasma. Exemplary tissue samples include choroid or retina.

The foregoing aspects and embodiments may be more fully understood by reference to the following detailed description and claims.

### BRIEF DESCRIPTION OF THE DRAWINGS

FIG. 1A depicts the transcript variant 1 mRNA sequence of human ROBO1 (SEQ ID NO: 1), which encodes isoform 1 of human ROBO1.

FIG. 1B depicts the transcript variant 2 mRNA sequence of human ROBO1 (SEQ ID NO: 2) which encodes isoform 2 of human ROBO1.

FIG. 1C depicts the transcript variant 4 mRNA sequence of human ROBO1 (SEQ ID NO: 3) which encodes isoform 4 of human ROBO1.

FIG. 1D depicts the isoform 1 amino acid sequence of human ROBO1 (SEQ ID NO: 4).

FIG. 1E depicts the isoform 2 amino acid sequence of human ROBO1 (SEQ ID NO: 5).

FIG. 1F depicts the isoform 4 amino acid sequence of human ROBO1 (SEQ ID NO: 6).

FIG. 2A depicts the transcript variant 1 mRNA sequence of human RORA (SEQ ID NO: 7), which encodes isoform a of RORA.

FIG. 2B depicts the transcript variant 2 mRNA sequence of human RORA (SEQ ID NO: 8) which encodes isoform b of RORA.

FIG. 2C depicts the transcript variant 3 mRNA sequence of human RORA (SEQ ID NO: 9) which encodes isoform c of RORA.

FIG. 2D depicts the transcript variant 4 mRNA sequence of human RORA (SEQ ID NO: 10) which encodes isoform d of RORA.

FIG. 2E depicts the isoform a amino acid sequence of human RORA (SEQ ID NO: 11).

FIG. 2F depicts the isoform b amino acid sequence of human RORA (SEQ ID NO: 12).

FIG. 2G depicts the isoform c amino acid sequence of human RORA (SEQ ID NO: 13).

FIG. 2H depicts the isoform d amino acid sequence of human RORA (SEQ ID NO: 14).

FIG. 3 provides a chart of genes that were identified as associated with certain biological functional categories using Ingenuity Pathway Analysis. Nine genes that were most significantly identified with tissue development include PLA2G4A, IL1A, MMP7, PKP2, CXCL13, IGHM, ENPP2, ROBO1, and RORA; the genes that were most significantly associated with lipid metabolism include PLA2G4A, IL1A, RORA, IGHM and ENPP2; the genes most significantly associated with neurological disease include UCHL1, PLA2G4A, IL1A, RORA, IGHM, ENPP2 and RGS13; the genes most significantly associated with carbohydrate metabolism include PLA2G4A, MMP7, IL1A, IGHM and ENPP2; the genes most significantly associated with immunological disease include PLA2G4, IL1A, CXCL13, RORA, IGHM, ENPP2, RPS6KA2, RGS13, NLRP2 and ROBO1; the genes most significantly associated with cardiovascular disease include PLA2G4A, MMP7, IL1A, PKP2, RORA, RGS13, RPS6KA2, and ROBO1; and the genes most significantly associated with cell death include PLA2G4A, IL1A, MMP7, IGHM, RPS6KA2, and RORA.

FIG. 4 provides a schematic drawing of a network of genes and pathways associated with AMD. ROBO1, RORA, NLRP2, PLA2G4A, and PKP2 are down-regulated in affected siblings compared to unaffected siblings while CXCL13, RGS13, RPS6KA2, IL1A, IL1/IL6/TNF, and MMP7 are up-regulated in affected siblings compared to unaffected siblings. Solid lines indicate direct relationships and dotted lines indicate indirect relationships as identified in previously published literature (www.ingenuity.com/index.html). The individual shapes represent the family of molecule, for example, the shape of RORA (highlighted in a box) indicates a ligand-dependent nuclear receptor.

FIG. 5 provides a table of 18 genes that were identified by gene expression studies as upregulated or downregulated in 9 sibling pairs wherein one individual was affected with AMD and the other sibling was unaffected.

FIG. 6 depicts linkage disequilibrium (r²) between SNPs from the ROBO1 gene for wet or dry AMD in NESC (A) and in GREEK (B) cohort, showing a minimum of three distinct haplotype blocks: the first block encompassing the region between rs1387665 and rs4264688, the second between rs6548621 to rs9826366, and the third block including rs3923526, rs9309833, and rs7629503.

FIG. 7 depicts association results of ROBO1 SNPs for wet AMD in the NESC and GREEK cohorts, and in meta-analysis using an additive model. Alleles were provided from the plus (+) strand using the NCBI B36 assembly of dbSNP b126.

FIG. 8 depicts association results of ROBO1 SNPs for dry AMD in the NESC and GREEK cohorts, and in meta-analysis using an additive model. Alleles were provided from the plus (+) strand using the NCBI B36 assembly of dbSNP b126.

FIG. 9 depicts significant haplotypes in RORA for wet AMD in the NESC, GREEK, NHS-HPFS cohorts, and in meta-analysis using an additive model. Alleles were provided from the plus (+) strand using the NCBI B36 assembly of dbSNP b126.

FIG. 10 depicts a summary of interaction analysis of ROBO1 SNPs (rs4513416, rs7640053, rs7622444 and rs9309833) and a RORA SNP (rs8034864) for wet and dry AMD in the three cohorts, NESC, GREEK, NHS-HPFS, and in meta-analysis. Alleles were provided from the plus (+) strand using the NCBI B36 assembly of dbSNP b126.

FIG. 11 depicts estimated probabilities for different categories of genotypes between ROBO1 SNPs and a RORA SNP in meta-analysis. The X-axis shows the categories of genotypes for rs8034864 from the RORA gene, and the Y-axis shows the estimated probabilities of different genotypic groups for rs4513416 (A and B) and rs9309833 (B and C) from the ROBO1 gene after adjusting for covariates. Graphs for wet AMD are shown in A and C, and for dry AMD in B and D. Alleles were provided from the plus (+) strand using the NCBI B36 assembly of dbSNP b126.

FIG. 12 depicts RNA expression of ROBO1 in the macula and extramacula from normal donors and donors with AMD. Absolute expression of ROBO1 in the RPE-Choroid is plotted on the Y-axis. Values for the macula and extra macula are plotted for both normal eyes and eyes with all AMD subtypes.

### DETAILED DESCRIPTION

As discussed previously, the methods and compositions disclosed herein are based, in part, upon the discovery of protective and risk variants and protective and risk haplotypes of the ROBO1 gene that are significantly associated with AMD risk. In some embodiments, variants, T>G (rs7615149) and C>T (rs59931439), C>T (rs1387665), T>C (rs9309833), and G>A (rs4513416) in the ROBO1 gene, have been found to be associated with risk of developing of AMD as determined by statistical analysis, haplotype analysis, or by virtue of the fact that they cluster with variants at polymorphic sites identified by statistical or haplotype analysis.

In addition, one haplotype in ROBO1 associated with a reduced risk of developing the neovascular form of AMD. This protective haplotype is defined by the polymorphic sites rs6548621 and rs7615149.

Although the polymorphic sites ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809 are known, their association with the risk of developing AMD (dry and/or neovascular AMD), as determined by statistical analysis, haplotype analysis, or by virtue of the fact that they cluster with variants at polymorphic sites identified by statistical or haplotype analysis, heretofore were not known.

ROBO1 is a member of the immunoglobulin gene superfamily and encodes an integral membrane protein that functions in axon guidance and neuronal precursor cell migration. This receptor is activated by SLIT-family proteins, resulting in a repulsive effect on glioma cell guidance in the developing brain.

As used herein, the term “ROBO1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the ROBO1 gene reported in the NCBI gene database (at website www.ncbi.nlm.nih.gov) under gene ID: 6091, gene location accession no. NC_{—}000003.11 (78646389..79639060, complement) or a strand complementary thereto; (ii) the full length sequence of the ROBO1 gene reported in the NCBI gene database under gene ID: 6091, gene location accession no. NC_{—}000003.11 (78646389..79639060, complement); (iii) a naturally occurring allelic variant of one of the foregoing sequences; or (iv) a nucleic acid sequence complementary to one of the foregoing sequences. The ROBO1 gene may also include upstream regulatory regions including promoter, enhancer and silencing regions of ROBO1 including one or more of the following allelic variants: rs7629503, rs9309833, rs10865579, rs1393370, rs3923526, rs6548621, rs7615149. The ROBO1 gene may also include intronic sequences and downstream regulatory regions.

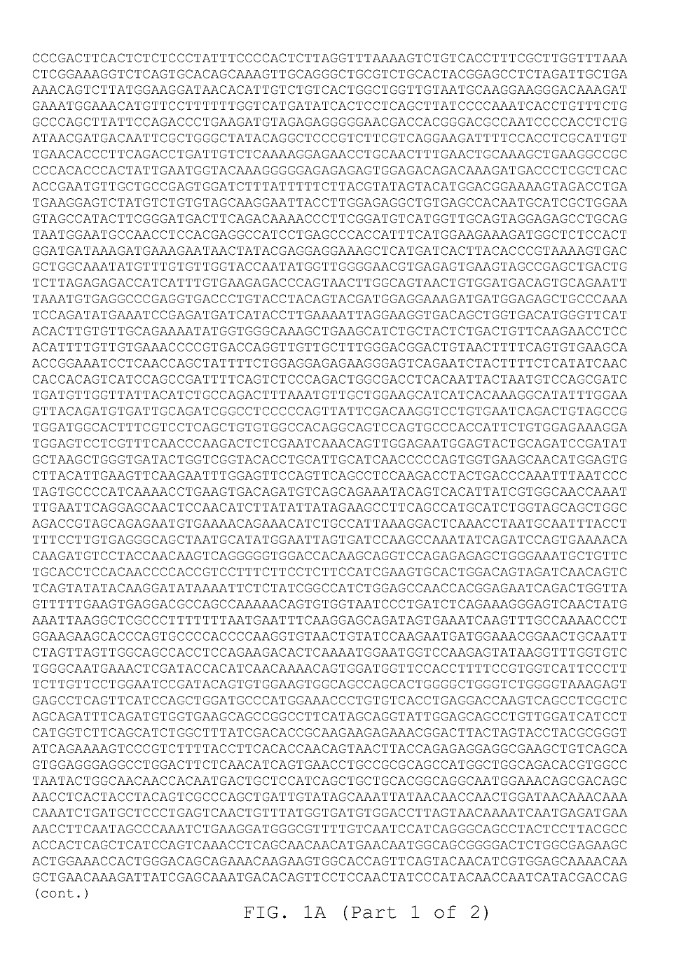

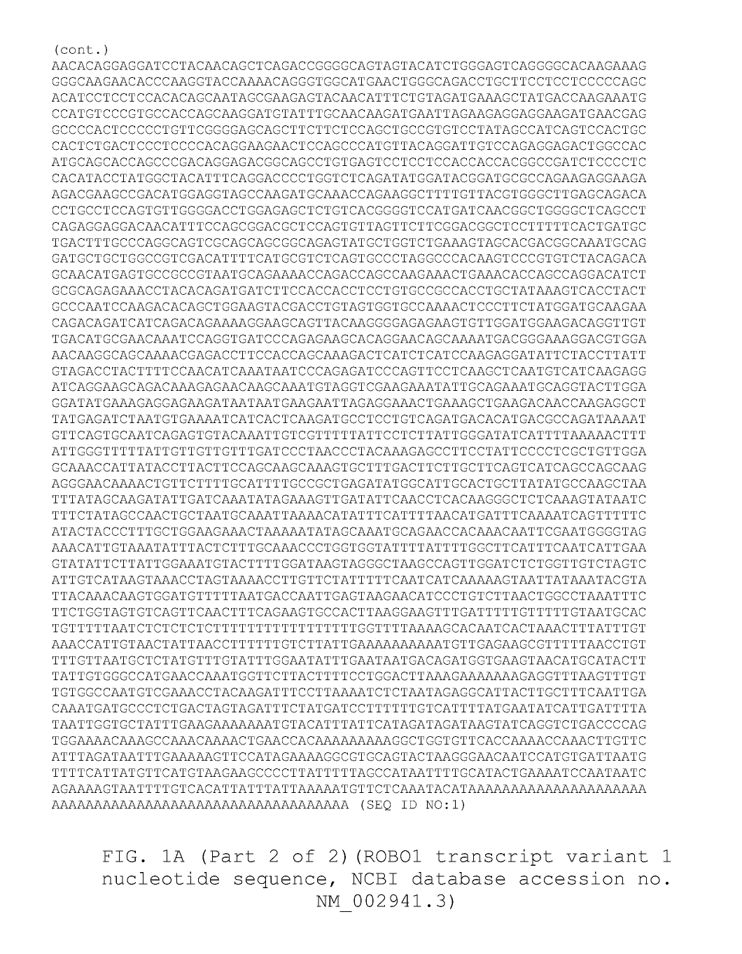

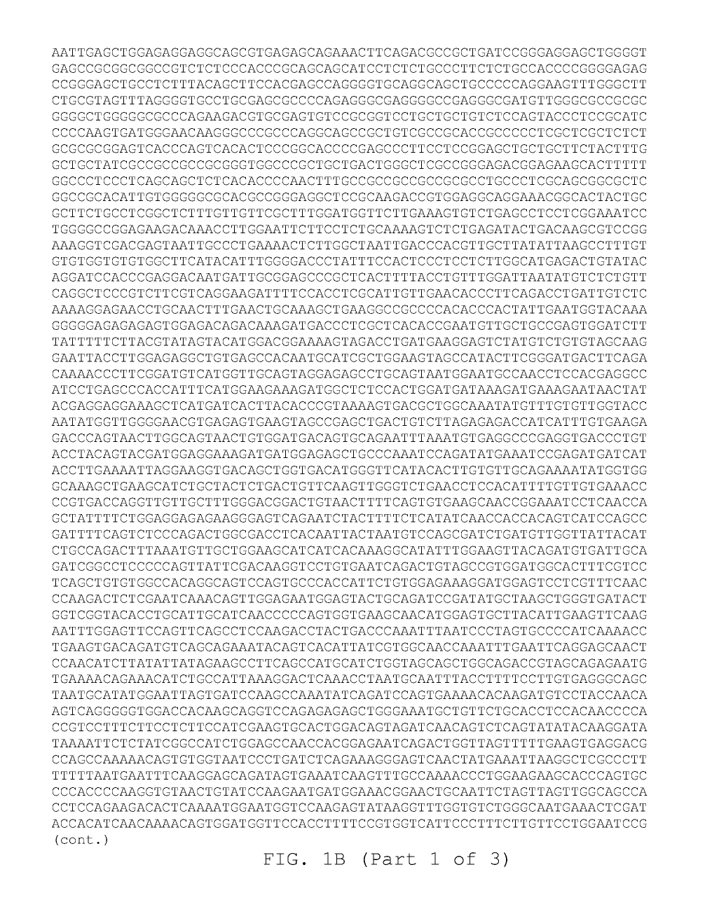

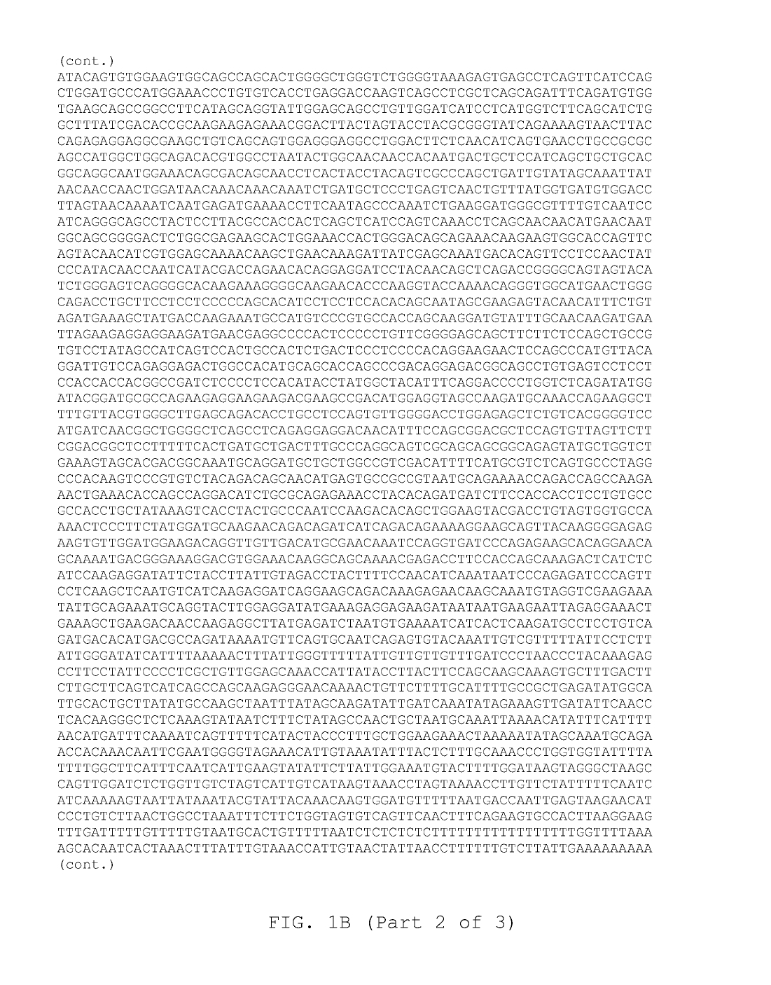

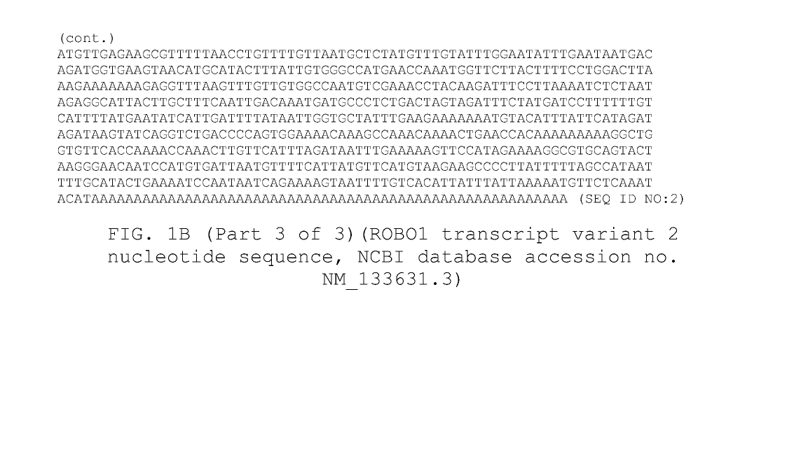

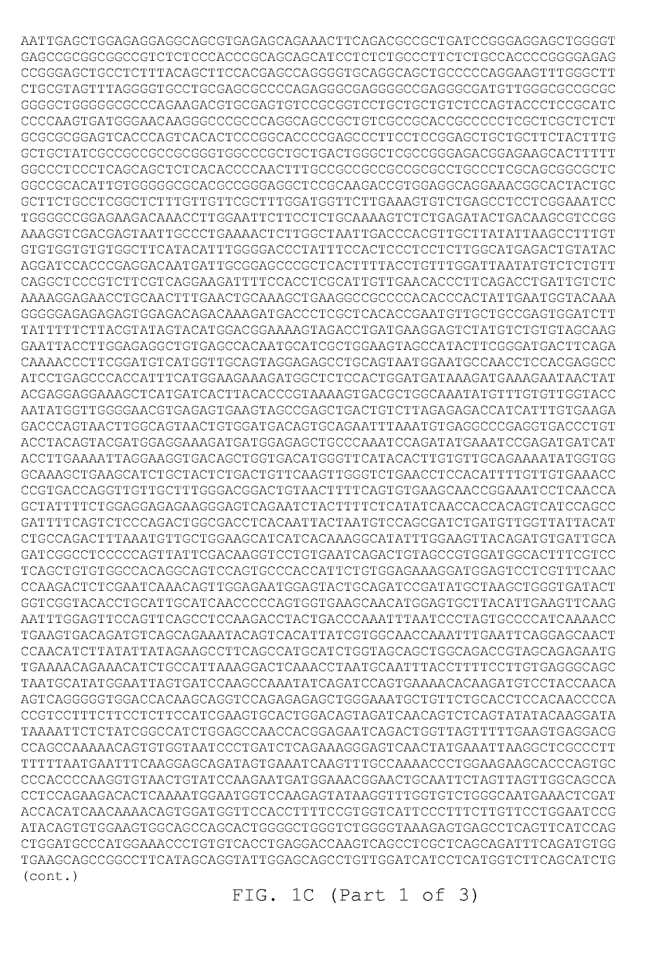

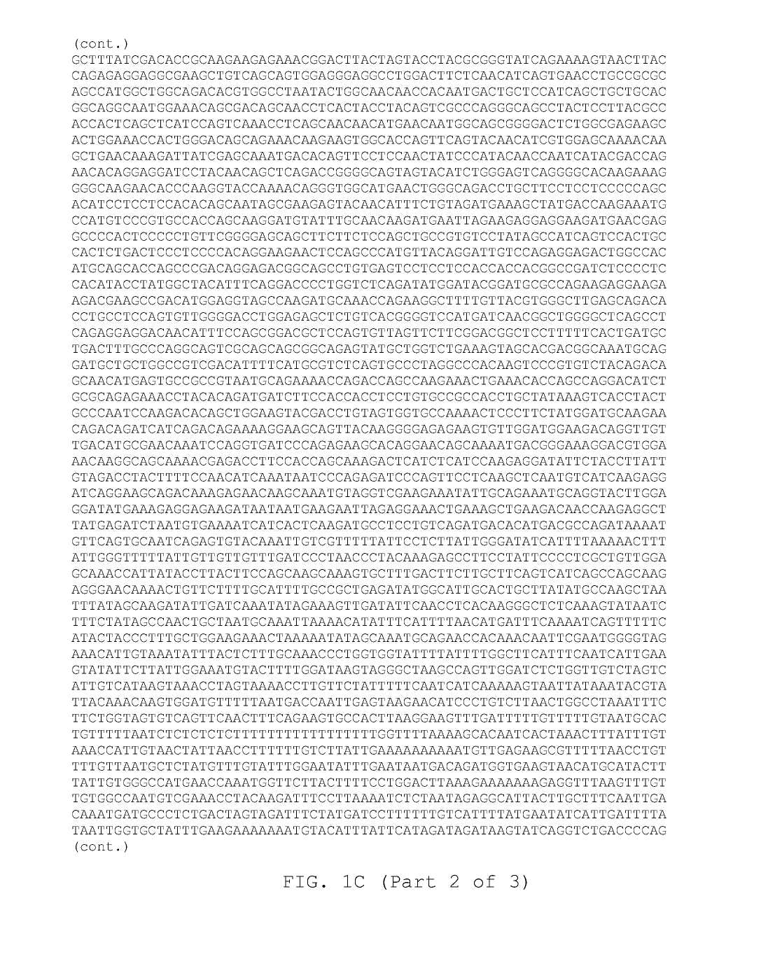

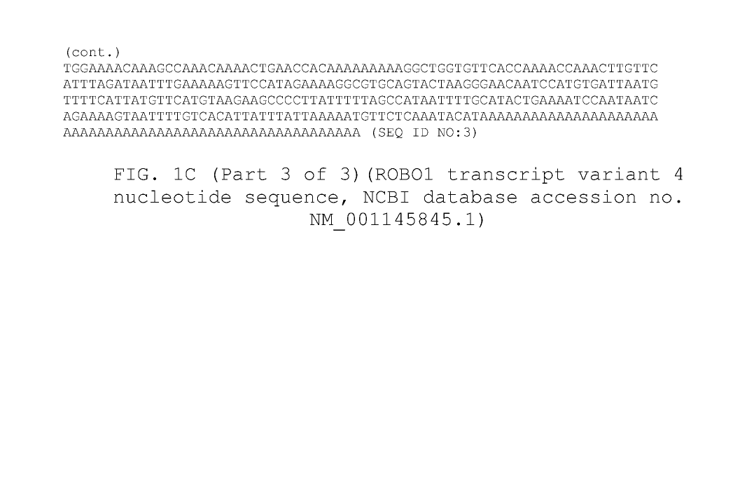

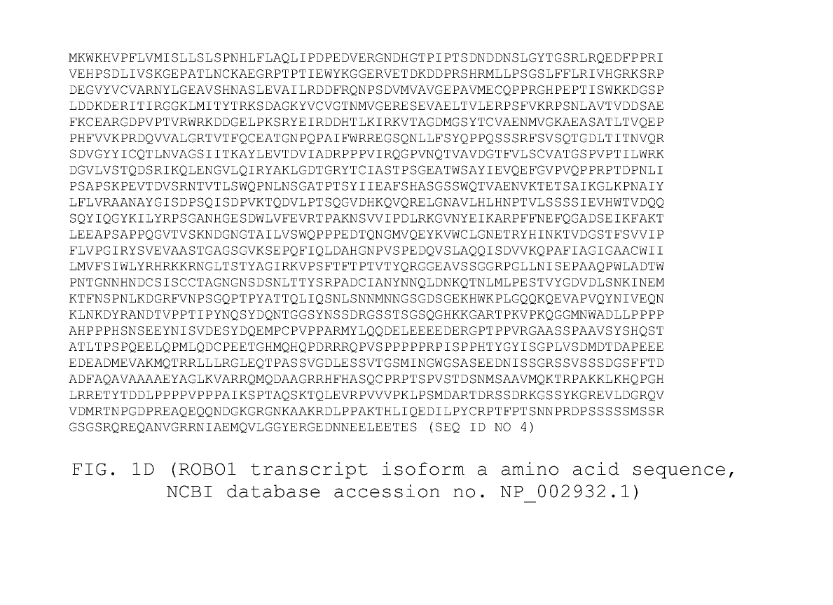

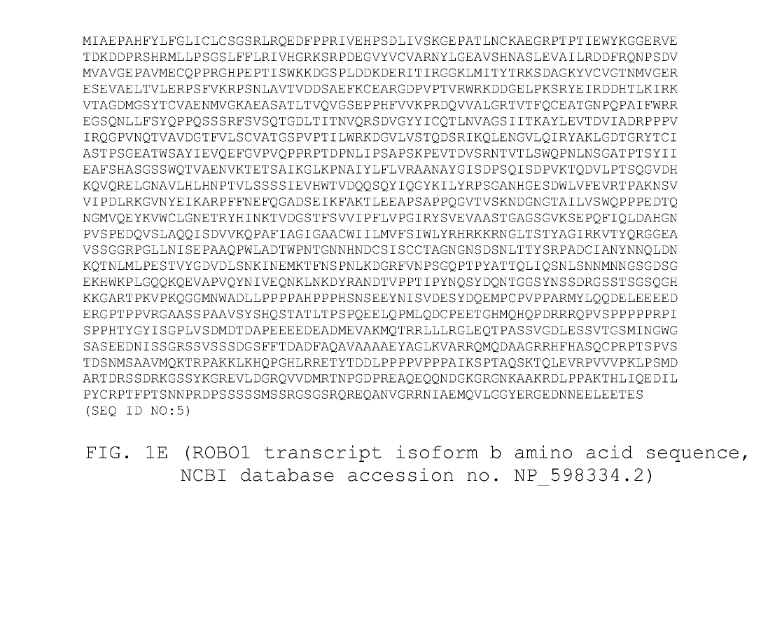

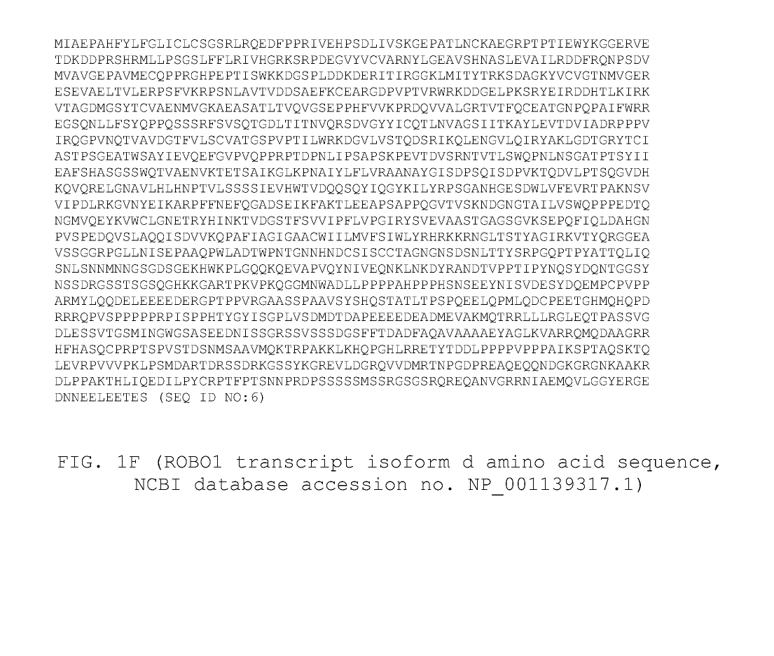

As used herein, a “ROBO1 gene product” is understood to mean (i) a nucleic acid sequence at least 75, at least 150, or at least 225 nucleotides in length that hybridizes under specific hybridization and washing conditions to the ROBO1 gene (either the sense or anti-sense sequence); (ii) a nucleic acid sequence that is at least 90%, more preferably at least 95%, and more preferably at least 98% identical to the mRNA sequence shown in one of FIGS. 1A-C, or a nucleic acid sequence that hybridizes under specific hybridization and washing conditions to the sequence shown in one of FIGS. 1A-C; or (iii) a peptide or protein at least 25, at least 50, or at least 75 amino acids in length that is at least 95%, more preferably at least 98%, and more preferably at least 99% identical to the amino acid sequence shown in one of FIGS. 1D-F.

Homology or identity is determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36, 290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases see Altschul et al., (1994) Nature Genetics 6, 119-129 which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89, 10915-10919, fully incorporated by reference). Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The nucleic acid encoding the human ROBO1 gene spans approximately 1,170,672 base pairs in length as reported in the NCBI gene database under gene ID: 6091, gene location accession no. NC_{—}000003.11 (78646389..79639060, complement). The gene is located on chromosome 3p12. The ROBO1 gene has been reported to generate at least three splicing transcript variants. Transcript variant 1 comprises 33 exons as reported in the NCBI nucleotide database under accession no. NM_{—}002941.3; the protein encoded by transcript variant 1 is 1651 amino acids in length as reported in the NCBI protein database under accession no. NP_{—}002932.1. Transcript variant 2 comprises 33 exons as reported in the NCBI nucleotide database under accession no. NM_{—}133631.3; the protein encoded by transcript variant 2 is 1606 amino acids in length as reported in the NCBI protein database under accession no. NP_{—}598334.2. Transcript variant 4 comprises 33 exons as reported in the NCBI nucleotide database under accession no. NM_{—}001145845.1; the protein encoded by transcript variant 4 is 1551 amino acids in length as reported in the NCBI protein database under accession no. NP_{—}001139317.1. Polymorphisms have been identified in the coding regions and untranslated regions of the exons, as well as in the introns and in the chromosome outside of the transcript region or regions of the ROBO1 gene. As examples of the polymorphisms in the ROBO1 gene, the NCBI SNP database reports 6989 specific polymorphic sites for the ROBO1 gene under gene ID: 6091. The mRNA sequences and the amino acid sequences of ROBO1 are set forth in FIGS. 1A-C and in FIGS. 1D-F, respectively.

### I. DEFINITIONS

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms.

A “polymorphic site” is the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in loci identified herein, including intragenic SNPs in exons, introns, or upstream or downstream regions of a gene (e.g., a promoter or enhancer), as well as SNPs that are located outside of gene sequences. Examples of such SNPs include, but are not limited to ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809.

The term “genotype” as used herein denotes one or more polymorphisms of interest found in an individual, for example, within a gene of interest. Diploid individuals have a genotype that comprises two different sequences (heterozygous) or one sequence (homozygous) at a polymorphic site.

The term “haplotype” refers to a DNA sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms on chromosome 3 near the ROBO1 gene, e.g. within the gene and/or within intergenic sequences (i.e., intervening intergenic sequences, upstream sequences, and downstream sequences that are in linkage disequilibrium with polymorphisms in the genic region). The term “haplotype” can refer to a set of single nucleotide polymorphisms (SNPs) found to be statistically associated on a single chromosome. A haplotype can also refer to a combination of polymorphisms (e.g., SNPs) and other genetic markers found to be statistically associated on a single chromosome. A haplotype, for instance, can also be a set of maternally inherited alleles, or a set of paternally inherited alleles, at any locus.

The term “genetic profile,” as used herein, refers to a collection of one or more polymorphic sites including ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809, optionally in combination with other genetic characteristics such as deletions, additions or duplications, and optionally combined with other polymorphic sites associated with AMD risk or protection. Thus, a genetic profile, as the phrase is used herein, is not limited to a set of characteristics defining a haplotype, and may include polymorphic sites from diverse regions of the genome. For example, a genetic profile for AMD includes one or a subset of single nucleotide polymorphisms such as ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809, optionally in combination with other genetic characteristics associated with AMD. It is understood that while one polymorphic site in a genetic profile may be informative of an individual's increased or decreased risk (i.e., an individual's propensity or susceptibility) to develop AMD, more than one polymorphic site in a genetic profile may and typically will be analyzed and will be more informative of an individual's increased or decreased risk of developing AMD. A genetic profile may include at least one SNP disclosed herein in combination with other polymorphisms or genetic markers and/or environmental factors (e.g., smoking or obesity) known to be associated with AMD. In some cases, a polymorphic site may reflect a change in regulatory or protein coding sequences that change gene product levels or activity in a manner that results in increased likelihood of development of disease. In addition, it will be understood by a person of skill in the art that one or more polymorphic sites that are part of a genetic profile may be in linkage disequilibrium with, and serve as a proxy or surrogate marker for, another genetic marker or polymorphism that is causative, protective, or otherwise informative of disease.

The term “gene,” as used herein, refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions).

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs. In certain embodiments, an allele is defined by the genotypes of at least 1, 2, 4, 8 or 16 or more SNPs, (including, but not limited to, ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809) in a gene.

A “causative” polymorphic site is a polymorphic site (e.g., a SNP) having an allele that is directly responsible for a difference in risk of development or progression of AMD. Generally, a causative polymorphic site has an allele producing an alteration in gene expression or in the expression, structure, and/or function of a gene product, and therefore is most predictive of a possible clinical phenotype. One such class includes polymorphic sites falling within regions of genes encoding a polypeptide product, i.e. “coding polymorphic sites” (e.g., “coding SNPs” (cSNPs)). These polymorphic sites may result in an alteration of the amino acid sequence of the polypeptide product (i.e., non-synonymous codon changes) and give rise to the expression of a defective or other variant protein. Furthermore, in the case of nonsense mutations, a polymorphic site may lead to premature termination of a polypeptide product. Such variant products can result in a pathological condition, e.g., genetic disease. Examples of genes in which a polymorphic site within a coding sequence causes a genetic disease include sickle cell anemia and cystic fibrosis.

Causative polymorphic sites do not necessarily have to occur in coding regions; causative polymorphic sites can occur in, for example, any genetic region that can ultimately affect the expression, structure, and/or activity of the protein encoded by a nucleic acid. Such genetic regions include, for example, those involved in transcription, such as polymorphic sites in transcription factor binding domains, polymorphic sites in promoter regions, in areas involved in transcript processing, such as polymorphic sites at intron-exon boundaries that may cause defective splicing, or polymorphic sites in mRNA processing signal sequences such as polyadenylation signal regions. Some polymorphic sites that are not causative polymorphic sites nevertheless are in close association with, and therefore segregate with, a disease-causing sequence. In this situation, the presence of an allele at the polymorphic site correlates with the presence of, or predisposition to, or an increased risk in developing the disease. These polymorphic sites, although not causative, are nonetheless also useful for diagnostics, disease predisposition screening, and other uses.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers may be present within the same gene or gene cluster.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium is influenced by a number of factors including genetic linkage, the rate of recombination, the rate of mutation, random drift, non-random mating, and population structure. “Linkage disequilibrium” or “allelic association” thus means the preferential association of a particular allele or genetic marker with another specific allele or genetic marker more frequently than expected by chance for any particular allele frequency in the population. A marker in linkage disequilibrium with a risk or protective variant, such as those at ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809, can be useful in detecting susceptibility to disease. A polymorphic variant that is in linkage disequilibrium with a causative, risk-associated, protective, or otherwise informative polymorphic variant or genetic marker is referred to as a “proxy” or “surrogate” polymorphic variant. A proxy polymorphic variant may be in at least 50%, 60%, or 70% in linkage disequilibrium with the causative polymorphic variant, and preferably is at least about 80%, 90%, and most preferably 95%, or about 100% in LD with the genetic marker.

A “nucleic acid,” “polynucleotide,” or “oligonucleotide” is a polymeric form of nucleotides of any length, may be DNA or RNA, and may be single- or double-stranded. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

“Hybridization probes” are nucleic acids capable of binding in a base-specific manner to a complementary strand of nucleic acid. Such probes include nucleic acids and peptide nucleic acids. Hybridization is usually performed under stringent conditions which are known in the art. A hybridization probe may include a “primer.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry and the instruments available from Applied Biosystems, Inc. (Foster City, Calif.); DuPont (Wilmington, Del.); or Milligen (Bedford, Mass.). When desired, the nucleic acids can be labeled using methodologies well known in the art such as described in U.S. Pat. Nos. 5,464,746; 5,424,414; and 4,948,882 all of which are herein incorporated by reference. In addition, the nucleic acids can comprise uncommon and/or modified nucleotide residues or non-nucleotide residues, such as those known in the art.

“Stringent” as used herein refers to hybridization and wash conditions at 50° C. or higher. Other stringent hybridization conditions may also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors may significantly affect the stringency of hybridization, including, among others, base composition, length of the nucleic acid strands, the presence of organic solvents, and the extent of base mismatching, the combination of parameters is more important than the absolute measure of any one.

The terms “susceptibility” and “risk” refer to either an increased or decreased likelihood of an individual developing a disorder (e.g., a condition, illness, disorder or disease) relative to a control and/or non-diseased population or to progressing from one form of a disorder to another relative to a control and/or a population having the initial form of the disorder. In one example, the control population may be individuals in the population (e.g., matched by age, gender, race and/or ethnicity) without the disorder, or without the genotype or phenotype assayed for. In another example, the control population may be individuals with the dry form of AMD (e.g., matched by age, gender, race and/or ethnicity), such as when considering risk of progressing from the dry form of AMD to the wet form of AMD.

The terms “diagnose” and “diagnosis” refer to the ability to determine or identify whether an individual has a particular disorder (e.g., a condition, illness, disorder or disease). The term “prognose” or “prognosis” refers to the ability to predict the course of the disease (including to predict the risk of developing the disease) and/or to predict the likely outcome of a particular therapeutic or prophylactic strategy.

The term “screen” or “screening” as used herein has a broad meaning. It includes processes intended for diagnosing or for determining the susceptibility, propensity, risk, or risk assessment of an asymptomatic subject for developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various polymorphic variants disclosed herein (and typically other polymorphic variants and genetic or behavioral characteristics) so as to determine where the individual lies on the spectrum of disease risk-neutrality-protection. Proxy polymorphic variants may substitute for any of these polymorphic variants. A sample such as a blood sample may be taken from the individual for purposes of conducting the genetic testing using methods known in the art or yet to be developed. Alternatively, if a health provider has access to a pre-produced data set recording all or part of the individual's genome (e.g. a listing of polymorphic variants in the individual's genome), screening may be done simply by inspection of the database, optimally by computerized inspection. Screening may further comprise the step of producing a report identifying the individual and the identity of alleles at the site of at least one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809 SNPs.

As used herein, the term “control value” means the level of gene expression or an amount of a gene product for a given gene of interest in a patient without AMD. By way of example, a ROBO1 gene product from a subject at risk of developing, or a subject who has, AMD is compared against the level of expression of a ROBO1 gene product in a subject without AMD (i.e., the control value for a ROBO1 gene product). In another example, a RORA gene product from a subject at risk of developing, or a subject who has, AMD is compared against the level of expression of a RORA gene product in a subject without AMD (i.e., the control value for a RORA gene product).

### II. PROGNOSIS AND DIAGNOSIS OF AMD BY DETECTING SINGLE NUCLEOTIDE POLYMORPHISMS

In one aspect, disclosed herein is a method of determining a subject's, for example, a human subject's, risk of developing age-related macular degeneration (AMD). The method comprises detecting in a sample, for example, a tissue, body fluid, or cell-containing sample, from a subject the presence or absence of an allelic variant at a polymorphic site of the ROBO1 gene that is associated with risk of developing AMD, such as a protective variant or a risk variant. In an exemplary embodiment, the method comprises determining whether the subject has a protective variant at a polymorphic site of the ROBO1 gene, wherein, if the subject has at least one protective variant, the subject is less likely to develop age-related macular degeneration than a subject without the protective variant. An exemplary protective variant is located in the promoter region of the ROBO1 gene.

In one exemplary embodiment, a protective variant T>G (rs7615149) in the ROBO1 gene was identified as associated with decreased risk of developing AMD. Throughout the specification, protective and risk variants are referred to using the following exemplary designation “T>G (rs7615149).” Using this convention, the first nucleotide base refers to the common allele (also referred to as the major allele) followed the “>” symbol then the variant allele (also referred to as the minor allele or rare allele). In some instances, the polymorphic site designation is provided in parentheses. It is contemplated herein that the skilled person would understand that the common and variant allele may be detected on either the forward or reverse strand of DNA. In some instances, the common and variant alleles and surrounding sequence provided herein were obtained from the forward strand as indicated in the Ensembl DNA database and in other instances the common and variant alleles and surrounding sequence provided herein were obtained from the forward strand as indicated in the NCBI DNA database, which is the reverse or reverse complement of the forward strand provided by Ensembl.

It is further contemplated herein that the skilled person would understand, based on a reference to the particular database, which allelic variants are relevant for a polymorphic site. In each of the foregoing embodiments, allelic variation maybe detected using the forward strand as indicated in the Ensembl DNA database or the forward strand as indicated and the NCBI DNA database.

In other embodiments, variants may be determined at the following polymorphic sites: rs6548621, rs7629503, rs9309833, rs10865579, rs1393370, rs3923526, rs59931439, rs13076006, rs7622444, rs6548625, rs7637338, 4513416, and rs1387665 in the ROBO1 gene, as described herein. In each of the embodiments below, the allelic variants at the denoted polymorphic sites are disclosed using the forward strand of the Ensembl database, unless otherwise indicated.

In an exemplary embodiment, the method comprises determining whether the subject has a protective variant at a polymorphic site of the ROBO1 gene, wherein if the subject has at least one protective variant, the subject is less likely to develop AMD than a subject without the protective variant. In one embodiment, a protective variant C>T (rs6548621) in the ROBO1 gene was identified as associated with decreased risk of developing wet AMD. In another embodiment, a protective variant C>T (rs59931439) in the ROBO1 gene was identified as associated with decreased risk of developing AMD. In another embodiment, a protective variant T>G (rs13076006) in the ROBO1 gene was identified as associated with decreased risk of developing wet AMD. In another embodiment, a protective variant A>G (rs6548625) in the ROBO1 gene was identified as associated with decreased risk of developing AMD. In another embodiment, a protective variant G>A (rs1393370) in the ROBO1 gene was identified as associated with decreased risk of developing AMD.

In an exemplary embodiment, the method comprises determining whether the subject has a risk variant at a polymorphic site of the ROBO1 gene, wherein if the subject has at least one risk variant, the subject is more likely to develop AMD than a subject without the risk variant. In one embodiment, a risk variant C>A (rs7629503) in the ROBO1 gene was identified as associated with increased risk of developing dry AMD. In another embodiment, a risk variant T>C (rs9309833) in the ROBO1 gene was identified as associated with increased risk of developing wet and/or dry AMD. However, when present in combination with variant G>A (rs8034864) of the RORA gene, risk variant T>C (rs9309833) in the ROBO1 gene was associated with decreased risk of developing wet and/or dry AMD. In another embodiment, a risk variant T>A (rs3923526) in the ROBO1 gene was identified as associated with increased risk of developing dry AMD. In another embodiment, a risk variant T>C (rs7622444) in the ROBO1 gene was identified as associated with increased risk of developing wet AMD. In another embodiment, a risk variant C>T (rs7637338) in the ROBO1 gene was identified as associated with increased risk of developing wet AMD. In another embodiment, a variant G>A (rs4513416) in the ROBO1 gene was identified as associated with risk of developing AMD. When present in combination with variant G>A (rs8034864) of the RORA gene, variant G>A (rs4513416) in the ROBO1 gene was associated with increased risk of developing dry AMD. In another embodiment, a risk variant C>T (rs1387665) in the ROBO1 gene was identified as associated with increased risk of developing AMD.

In another embodiment, a variant T>C (rs10865579) in the ROBO1 gene was identified as associated with the risk of developing AMD.

In each of the foregoing embodiments, the skilled person would understand that the allelic variants for each disclosed polymorphism could also be denoted using the reverse-complement sequence of the Ensembl DNA database, which corresponds to the forward sequence of the NCBI DNA database. For example, when the NCBI database is used, risk variant A>G (rs9309833) in the ROBO1 gene is associated with increased risk of developing wet and/or dry AMD. However, when present in combination with variant C>T (rs8034864) of the RORA gene, risk variant A>G (rs9309833) in the ROBO1 gene was associated with decreased risk of developing wet and/or dry AMD. In another example, when the NCBI database is used, variant C>T (rs4513416) in the ROBO1 gene was identified as associated with risk of developing AMD. When present in combination with variant C>T (rs8034864) of the RORA gene, variant C>T (rs4513416) in the ROBO1 gene was associated with increased risk of developing dry AMD. In another example, when the NCBI database is used, a risk variant G>A (rs1387665) in the ROBO1 gene was identified as associated with increased risk of developing AMD.

Exemplary sequences for variants in the ROBO1 gene are disclosed below. An exemplary protective variant is at a SNP, rs7615149 located in the promoter region of the ROBO1 gene. For example, the forward sequence comprises TAGACTCATATAACCATAACACAACCCAAGAATATTAATATCAGAGAGTATTTATA AGTGAAAAAGATGTCAATTTTCCTAATGAGTTTGAAAATATTGTATGGTATAAT[X₁₅]CTGAGACAGCAATTCAGATTTTTAAAAATCATACCATAGACGAGTACTTTGGTTTT TATGATTTCTATTCTTTTTATTGGTCACAGTTGTTTTATCACACACTGGAAATT (SEQ ID NO: 15) wherein X₁₅ is a thymine to a guanine substitution. T is the common allele, and G is the protective variant. Alternatively, the reverse complement sequence comprises AATTTCCAGTGTGTGATAAAACAACTGTGACCAATAAAAAGAATAGAAATCATAA AAACCAAAGTACTCGTCTATGGTATGATTTTTAAAAATCTGAATTGCTGTCTCAG[X₁₆]ATTATACCATACAATATTTTCAAACTCATTAGGAAAATTGACATCTTTTTCACTT ATAAATACTCTCTGATATTAATATTCTTGGGTTGTGTTATGGTTATATGAGTCTA (SEQ ID NO: 16) wherein X₁₆ is an adenine to a cytosine substitution. A is the common allele, and C is the protective variant. rs7615149 is a single nucleotide polymorphism with a T to a G substitution in the forward sequence or an A to a C substitution in the reverse complement sequence at chromosome 3 base pair position 79537773 in Ensembl Build 37.

Another protective variant is at a SNP, rs6548621, located in the promoter region of the ROBO1 gene. For example, the forward sequence comprises GTGAAAAAGTCATTGAGGTGGTGCTTCGTGAACTAGTTAAGAAAATAAAAATTCTG TAGGGCAGAGGTAGGCAAACATTGGCTAGACTTTGAGGACCATCCATTCTCTGT[X_{1 7}]ACTACATCTCAAAAACCATAGAACAGCAACATTTTGAAAATAATACAGCCATAG TCAATAGATAAACAAATGAGTGTGATAGTTTTCCAATAAAAAATGACTTATAAAAA (SEQ ID NO: 17) wherein X₁₇ is a cytosine to a thymine substitution. C is the common allele, and T is the variant allele. Alternatively, the reverse complement sequence comprises TTTTTATAAGTCATTTTTTATTGGAAAACTATCACACTCATTTGTTTATCTATTGACT ATGGCTGTATTATTTTCAAAATGTTGCTGTTCTATGGTTTTTGAGATGTAGT[X₁₈]AC AGAGAATGGATGGTCCTCAAAGTCTAGCCAATGTTTGCCTACCTCTGCCCTACAGA ATTTTTATTTTCTTAACTAGTTCACGAAGCACCACCTCAATGACTTTTTCAC (SEQ ID NO: 18) wherein X₁₈ is a guanine to an adenine substitution. G is the common allele, and A is the variant allele. rs6548621 is a single nucleotide polymorphism with a C to a T substitution in the forward sequence or a G to an A substitution in the reverse complement sequence at chromosome 3 base pair position 79550373 in Ensembl Build 37.

Another protective variant is at a SNP, rs59931439 located in intron 2 of the ROBO1 gene. For example, the forward sequence comprises TGTAGTCAAGGCGGACACCAGAAAGATTGTTAGTAAATAGGGTAGGAAGGCTAGG CCAATGTTATGCAGTGTTTAAATAGTAATGGTTAAGCCAATGCTTTAAAAATAAG[X₁₉]GATTAACTGTTTTCAAGTGATATACGAAGATATTTTGTGAATTCTTCTGCAGGC TCCCGTCTTCGTCAGGAAGATTTTCCACCTCGCATTGTTGAACACCCTTCAGACCT (SEQ ID NO: 19) wherein X₁₉ is a cytosine to a thymine substitution. C is the common allele, and T is the variant allele. Alternatively, the reverse complement sequence comprises AGGTCTGAAGGGTGTTCAACAATGCGAGGTGGAAAATCTTCCTGACGAAGACGGG AGCCTGCAGAAGAATTCACAAAATATCTTCGTATATCACTTGAAAACAGTTAATC[X₂₀]CTTATTTTTAAAGCATTGGCTTAACCATTACTATTTAAACACTGCATAACATTG GCCTAGCCTTCCTACCCTATTTACTAACAATCTTTCTGGTGTCCGCCTTGACTACA (SEQ ID NO: 20) wherein X₂₀ is a guanine to an adenine substitution. G is the common allele, and A is the variant allele. rs59931439 is a single nucleotide polymorphism with a C to a T substitution in the forward sequence or a G to an A substitution in the reverse complement sequence at chromosome 3 base pair position 78988130 in Ensembl Build 37.

Another protective variant is at a SNP, rs13076006 located in the promoter region of the ROBO1 gene. For example, the forward sequence comprises AATACAATGTCTTTGAAAAAGAAACGATGTCCAATTTTACTGTTCTTTAGTCCTTCT TAGAAACTACCTATTATTTGCCATTTGAAATTGTTCCTACGTTACAGAACTGT[X₂₁]A AAAATKTATGTGTTAGAACTCAGTTAGTTTTGGACAGCATAATGATGTAGAACAGT GTGTCTGAGGAAATATGGTGATGAATATATCACTGCTATAACTTGTCCAAAAT (SEQ ID NO: 21) wherein X₂₁ is a thymine to a guanine substitution. T is the common allele, and G is the variant allele. Alternatively, the reverse complement sequence comprises ATTTTGGACAAGTTATAGCAGTGATATATTCATCACCATATTTCCTCAGACACACTG TTCTACATCATTATGCTGTCCAAAACTAACTGAGTTCTAACACATAMATTTTT[X₂₂]A CAGTTCTGTAACGTAGGAACAATTTCAAATGGCAAATAATAGGTAGTTTCTAAGAA GGACTAAAGAACAGTAAAATTGGACATCGTTTCTTTTTCAAAGACATTGTATT (SEQ ID NO: 22) wherein X₂₂ is an adenine to a cytosine substitution. G is the common allele, and T is the variant allele. rs13076006 is a single nucleotide polymorphism with a T to a G substitution in the forward sequence or an A to a C substitution in the reverse complement sequence at chromosome 3 base pair position 79452636 in Ensembl Build 37.

Another protective variant is at a SNP, rs6548625 located in the promoter region of the ROBO1 gene. For example, the forward sequence comprises AGTAAAATATGTGATTCCATATTTGTAAAATRTTCTAAATGTTGAAATTCTTTTGAT AGACAGCAAAGGTACTTTAAGAACAAAAGCATGTTTCCTTAGATTCCATAAAA[X₂₃]TTCAATGAGTAGTTCATAATACTTAAGTGTTTATTTTAAATGTGTTCATTTTAGTGT CTGTGTTTGAAYTTGCTGAATGTATRCATTAAGCTACAATTTTATGGAAAACA (SEQ ID NO: 23) wherein X₂₃ is an adenine to a guanine substitution. A is the common allele, and G is the variant allele. Alternatively, the reverse complement sequence comprises TGTTTTCCATAAAATTGTAGCTTAATGYATACATTCAGCAARTTCAAACACAGACA CTAAAATGAACACATTTAAAATAAACACTTAAGTATTATGAACTACTCATTGAA[X_{2 4}]TTTTATGGAATCTAAGGAAACATGCTTTTGTTCTTAAAGTACCTTTGCTGTCTATC AAAAGAATTTCAACATTTAGAAYATTTTACAAATATGGAATCACATATTTTACT (SEQ ID NO: 24) wherein X₂₄ is a thymine to a cytosine substitution. T is the common allele, and C is the variant allele. rs6548625 is a single nucleotide polymorphism with an A to a G substitution in the forward sequence or a T to a C substitution in the reverse complement sequence at chromosome 3 base pair position 79563987 in Ensembl Build 37.

Another protective variant is at a SNP, rs1393370 located in the promoter region of the ROBO1 gene. For example, the forward sequence comprises CAGAATTACTCCATGGCTAATGGTTGGCTGAGGGAATTGACTAGGCTGATATGGTT TGTTCTGCTGAAAAAGATCTCCCATCCTGCAGCAGGTAGCCCTAGCTCCTTGGG[X₂₅]TTCCAAAGAACGGTAACAGAGCAAGCCCCTAAGCACAACCTTTTCCAGCTTCTTA TATCAAGTTTTCCAATATTTCCTTGGCAAAACTAAGTCTTATGGCCAACTCAAAA (SEQ ID NO: 25) wherein X₂₅ is a guanine to an adenine substitution. G is the common allele, and A is the variant allele. Alternatively, the reverse complement sequence comprises TTTTGAGTTGGCCATAAGACTTAGTTTTGCCAAGGAAATATTGGAAAACTTGATAT AAGAAGCTGGAAAAGGTTGTGCTTAGGGGCTTGCTCTGTTACCGTTCTTTGGAA[X₂₆]CCCAAGGAGCTAGGGCTACCTGCTGCAGGATGGGAGATCTTTTTCAGCAGAACAA ACCATATCAGCCTAGTCAATTCCCTCAGCCAACCATTAGCCATGGAGTAATTCTG (SEQ ID NO: 26) wherein X₂₆ is a cytosine to a thymine substitution. C is the common allele, and T is the variant allele. rs1393370 is a single nucleotide polymorphism with a G to an A substitution in the forward sequence or a C to a T substitution in the reverse complement sequence at chromosome 3 base pair position 79790293 in Ensembl Build 37.

An exemplary risk variant is at a SNP, rs7629503 located in the promoter region of the ROBO1 gene. For example, the forward sequence comprises CTATAGGAAATTGAGGTCCTAGAAGGCTAACTGACTAATTCAAAACTACATAGGAT AAAACTGTAGAAACAGTGTTAGTCACCGTACCTGCAATAGATATTTCACTTAAT[X₂₇]CCCACATAACCCTTTCAAAGTAGGCTTTATTAGATGTCTACAACACATGAAGAGA ATGAAGCTCAGAGAGTTTAAGGAAAATAGACATGACTATTCAGCCAAAAAGGGGC (SEQ ID NO: 27) wherein X₂₇ is a cytosine to an adenine substitution. C is the common allele, and A is the variant allele. Alternatively, the reverse complement sequence comprises GCCCCTTTTTGGCTGAATAGTCATGTCTATTTTCCTTAAACTCTCTGAGCTTCATTCT CTTCATGTGTTGTAGACATCTAATAAAGCCTACTTTGAAAGGGTTATGTGGG[X₂₈]A TTAAGTGAAATATCTATTGCAGGTACGGTGACTAACACTGTTTCTACAGTTTTATCC TATGTAGTTTTGAATTAGTCAGTTAGCCTTCTAGGACCTCAATTTCCTATAG (SEQ ID NO: 28) wherein X₂₈ is a guanine to a thymine substitution. G is the common allele, and T is the variant allele. rs7629503 is a single nucleotide polymorphism with a C to an A substitution in the forward sequence or a G to a T substitution in the reverse complement sequence at chromosome 3 base pair position 79813292 in Ensembl Build 37.

Another risk variant is at a SNP, rs9309833 located in the promoter region of the ROBO1 gene. For example, the forward sequence comprises ACTTGCATTTTCTTAAACACTCAGGATGTTTCATTCCTCTCGGCTTTTGTGTGTGTGT GTGTGTGTGTGTTTGTCCAGAATTCTGCCCCAAATGGTTCTCACTTTCTTAT[X₂₉]TTT TTAGCGATGTTTGAAAACACAAAACAAGTGTCACTTCTTCTGTGAAGACCTTCATG TTAAGAAAATAGGTTTAAGTATTCCTCCCTTTCTGATCATTTAATAATGCC (SEQ ID NO: 29) wherein X₂₉ is a thymine to a cytosine substitution. T is the common allele, and C is the variant allele. Alternatively, the reverse complement sequence comprises GGCATTATTAAATGATCAGAAAGGGAGGAATACTTAAACCTATTTTCTTAACATGA AGGTCTTCACAGAAGAAGTGACACTTGTTTTGTGTTTTCAAACATCGCTAAAAA[X₃₀]ATAAGAAAGTGAGAACCATTTGGGGCAGAATTCTGGACAAACACACACACACAC ACACACACAAAAGCCGAGAGGAATGAAACATCCTGAGTGTTTAAGAAAATGCAAG T (SEQ ID NO: 30) wherein X₃₀ is an adenine to a guanine substitution. A is the common allele, and G is the variant allele. rs9309833 is a single nucleotide polymorphism with a T to a C substitution in the forward sequence or an A to a G substitution in the reverse complement sequence at chromosome 3 base pair position 79811719 in Ensembl Build 37.

Another risk variant is at a SNP, rs3923526 located in the promoter region of the ROBO1 gene. For example, the forward sequence comprises GAGGTAATGTCTAAGTGGTCATTCATTCACACATGTAATTCACATATTCCATTCTGT ATCATTAGAAAATGGATTTTAATGCAAGAAGGGGTTGTTACGATTCAGAGCAC[X₃₁]GGCTCTCAAACTTTGCTACGTGTTAGAATCACCAAGGGAACTTTAACAATTTCAAT AACCAGGTAGCATCCAGACAAATTAAAACAATCTCCAAAAATGCCCAGGGTTAG (SEQ ID NO: 31) wherein X₃₁ is a thymine to an adenine substitution. T is the common allele, and A is the variant allele. Alternatively, the reverse complement sequence comprises CTAACCCTGGGCATTTTTGGAGATTGTTTTAATTTGTCTGGATGCTACCTGGTTATT GAAATTGTTAAAGTTCCCTTGGTGATTCTAACACGTAGCAAAGTTTGAGAGCC[X₃₂]GTGCTCTGAATCGTAACAACCCCTTCTTGCATTAAAATCCATTTTCTAATGATACAG AATGGAATATGTGAATTACATGTGTGAATGAATGACCACTTAGACATTACCTC (SEQ ID NO: 32) wherein X₃₂ is an adenine to a thymine substitution. A is the common allele, and T is the variant allele. rs3923526 is a single nucleotide polymorphism with a T to an A substitution in the forward sequence or an A to a T substitution in the reverse complement sequence at chromosome 3 base pair position 79784128 in Ensembl Build 37.

Another risk variant is at a SNP, rs7622444 located in the promoter region of the ROBO1 gene. For example, the forward sequence comprises AACTAAACAATTATATGCCAATAAAGCCCACATATTATAAATGTTTGTCTACAGAA TAAGAGAATAATGTGTAATTAACTTGACCAGCCTCCAACAAAACCCATGCTAAA[X₃₃]AGAAGAAGGTCACTTATTTTGATGAGCAGACTCTAATTGCTTCATTTATATTTTT GATTTTTTCTCAGAGATAATTAGAAAACGGATGCCRGATCCTGCATTCTGTTTTA (SEQ ID NO: 33) wherein X₃₃ is a thymine to a cytosine substitution. T is the common allele, and C is the variant allele. Alternatively, the reverse complement sequence comprises TAAAACAGAATGCAGGATCYGGCATCCGTTTTCTAATTATCTCTGAGAAAAAATCA AAAATATAAATGAAGCAATTAGAGTCTGCTCATCAAAATAAGTGACCTTCTTCT[X_{3 4}]TTTAGCATGGGTTTTGTTGGAGGCTGGTCAAGTTAATTACACATTATTCTCTTATT CTGTAGACAAACATTTATAATATGTGGGCTTTATTGGCATATAATTGTTTAGTT (SEQ ID NO: 34) wherein X₃₄ is an adenine to a guanine substitution. A is the common allele, and G is the variant allele. rs7622444 is a single nucleotide polymorphism with a T to a C substitution in the forward sequence or an A to a G substitution in the reverse complement sequence at chromosome 3 base pair position 79557927 in Ensembl Build 37.

Another risk variant is at a SNP, rs7637338 located in the promoter region of the ROBO1 gene. For example, the forward sequence comprises TTTAAGCTCTATGGCCAACCTGTTGARCTAGGTGTCCTATCTACAGACTGAGTGTAT GAATGGGTGGAAACAAGATGATGAAAATTACAGAGAGAACTGAATTAGACAAC[X_{3 5}]AGTTATTTGAAAATGCATATCCTTCGAGAATAGTAGAAAGTAAGTAGAGAAATTT ACTAATATATCCATCCAAAGGAATCCAAATTTTCTTCCTTGAGTGAGTAGAGTAT (SEQ ID NO: 35) wherein X₃₅ is a cytosine to a thymine substitution. C is the common allele, and T is the variant allele. Alternatively, the reverse complement sequence comprises ATACTCTACTCACTCAAGGAAGAAAATTTGGATTCCTTTGGATGGATATATTAGTA AATTTCTCTACTTACTTTCTACTATTCTCGAAGGATATGCATTTTCAAATAACT[X₃₆]GTTGTCTAATTCAGTTCTCTCTGTAATTTTCATCATCTTGTTTCCACCCATTCATACA CTCAGTCTGTAGATAGGACACCTAGYTCAACAGGTTGGCCATAGAGCTTAAA (SEQ ID NO: 36) wherein X₃₆ is a guanine to an adenine substitution. G is the common allele, and A is the variant allele. rs7637338 is a single nucleotide polymorphism with a C to a T substitution in the forward sequence or a G to an A substitution in the reverse complement sequence at chromosome 3 base pair position 79560604 in Ensembl Build 37.

Another variant is at a SNP, rs4513416 located in the promoter region of the ROBO1 gene. For example, the forward sequence comprises CTTACACTAACACTCTGCAGACTCTAGAAAATGAGATTCGTTTTTTTCCTTTGACAC ACTGTTTGTGGAAGTGCCCCTGAGTCATATCATTATATCTAAGATGACCAATT[X₃₇]CTTTTTCTGAGGATAGAAATTCAAGATGAAGTTATTTGAAGGACTAAGGAGAGTAA TGATGAATTTTTCATATGYTCTTATTCTATTTTCTCGCTGTAAAAAATGTATAA (SEQ ID NO: 37) wherein X₃₇ is a guanine to an adenine substitution. G is the common allele, and A is the variant allele. Alternatively, the reverse complement sequence comprises TTATACATTTTTTACAGCGAGAAAATAGAATAAGARCATATGAAAAATTCATCATT ACTCTCCTTAGTCCTTCAAATAACTTCATCTTGAATTTCTATCCTCAGAAAAAG[X₃₈]AATTGGTCATCTTAGATATAATGATATGACTCAGGGGCACTTCCACAAACAGTGTG TCAAAGGAAAAAAACGAATCTCATTTTCTAGAGTCTGCAGAGTGTTAGTGTAAG (SEQ ID NO: 38) wherein X₃₈ is a cytosine to a thymine substitution. C is the common allele, and T is the variant allele. rs4513416 is a single nucleotide polymorphism with a G to an A substitution in the forward sequence or a C to a T substitution in the reverse complement sequence at chromosome 3 base pair position 79490803 in Ensembl Build 37.

Another risk variant is at a SNP, rs1387665 located in the promoter region of the ROBO1 gene. For example, the forward sequence comprises TCACAAGGCCAGCCTAGATTTAAGGGATGGGAAAATGGACTTCGGCTCTTGATGG GAGCAGTCTCAGTCGCATTGGRTAGGACACAACATAGGGAAGTCATTAATTCGGA[X₃₉]GATCAGTGGAATCAATCTACCATATTTTCAAATAATATGGTAGATTATGAYATT AATCTACCATATTAAAWTAAAATTTTGCTAACCTAAGAAAAGGTTAGCAAAATGC A (SEQ ID NO: 39) wherein X₃₉ is a cytosine to a thymine substitution. C is the common allele, and T is the variant allele. Alternatively, the reverse complement sequence comprises TGCATTTTGCTAACCTTTTCTTAGGTTAGCAAAATTTTAWTTTAATATGGTAGATTA ATRTCATAATCTACCATATTATTTGAAAATATGGTAGATTGATTCCACTGATCPC[X₄₀]T CCGAATTAATGACTTCCCTATGTTGTGTCCTAYCCAATGCGACTGAGACTGCTCCC ATCAAGAGCCGAAGTCCATTTTCCCATCCCTTAAATCTAGGCTGGCCTTGTGA (SEQ ID NO: 40) wherein X₄₀ is a guanine to an adenine substitution. G is the common allele, and A is the variant allele. rs1387665 is a single nucleotide polymorphism with a C to a T substitution in the forward sequence or a G to an A substitution in the reverse complement sequence at chromosome 3 base pair position 79429811 in Ensembl Build 37.

Another variant is at a SNP, rs10865579 located in the promoter region of the ROBO1 gene. For example, the forward sequence comprises TCCCCCATCAGAATTACTACAATAGAATATATGGGGGTGGGGCACTTGAGTCCACA TATTAACAGAATCTATTCCAGGTGTAACTAGGAACAGGGAGTTTATCACAACAA[X_{4 1}]TGCTCTCCAATTCAGTCAGATCAATATGGCACTTAATTTAGCATTTGGGGGAGGA GCCATTTGCAAAGCTTTTTAGATCTTATTTTGTGTCTTCCCAGATTACCGTGCTT (SEQ ID NO: 41) wherein X₄₁ is a thymine to a cytosine substitution. T is the common allele, and C is the variant allele. Alternatively, the reverse complement sequence comprises AAGCACGGTAATCTGGGAAGACACAAAATAAGATCTAAAAAGCTTTGCAAATGGC TCCTCCCCCAAATGCTAAATTAAGTGCCATATTGATCTGACTGAATTGGAGAGCA[X₄₂]AAGCACGGTAATCTGGGAAGACACAAAATAAGATCTAAAAAGCTTTGCAAATG GCTCCTCCCCCAAATGCTAAATTAAGTGCCATATTGATCTGACTGAATTGGAGAGC A (SEQ ID NO: 42) wherein X₄₂ is an adenine to a guanine substitution. A is the common allele, and G is the variant allele. rs10865579 is a single nucleotide polymorphism with a T to a C substitution in the forward sequence or an A to a G substitution in the reverse complement sequence at chromosome 3 base pair position 79811006 in Ensembl Build 37.

In another aspect, methods are provided for determining a subject's, for example, a human subject's, risk of developing age-related macular degeneration. The method comprises detecting in a sample from a subject the presence or absence of a haplotype in the ROBO1 gene. If the subject has a protective haplotype, the subject is less likely to develop age-related macular degeneration than a person without the protective haplotype. If the subject has a risk haplotype, the subject is more likely to develop age-related macular degeneration than a person without the risk haplotype.

In one embodiment, a haplotype is defined by the alleles present at the polymorphic sites rs6548621 and rs7615149. The method comprises detecting a cytosine or thymine base at rs6548621 and a guanine or thymine base at rs7615149. When the haplotype comprises a guanine in the forward sequence of rs7615149 and a cytosine or thymine in the forward sequence of rs6548621, the haplotype is a protective haplotype indicating that the subject is less likely to develop AMD than a person without this haplotype.

In some embodiments, a protective variant and/or a risk variant of the ROBO1 gene, and/or a protective haplotype and/or a risk haplotype of the ROBO1 gene may be detected in combination with a protective variant and/or a risk variant (and/or a protective and/or risk haplotype) at one or more of the following polymorphic sites: rs1061170 (CFH), rs800292 (CFH), rs10490924 (LOC387715), rs11200638 (ARMS2/HTRA1), rs2672598 (ARMS2/HTRA1), rs10664316 (ARMS2/HTRA1), rs1049331 (ARMS2/HTRA1), rs12900948 (RORA), rs4335725 (RORA), rs8034864 (RORA), and rs1045216 (PLEKHA1).

In one embodiment, a RORA haplotype is defined by the alleles present at the polymorphic sites rs12900948, rs730754, and rs8034864. The method comprises detecting an adenine base or guanine base at rs12900948, an adenine or guanine base at rs730754, and a cytosine base or adenine base at rs8034864. When the haplotype comprises an adenine in the forward sequence of rs12900948, an adenine in the forward sequence of rs730754, and a cytosine in the forward sequence of rs8034864, the haplotype is a risk haplotype indicating that the subject is more likely to develop AMD than a person without this haplotype.

In another embodiment, a RORA haplotype is defined by the alleles present at the polymorphic sites rs17237514 and rs4335725. The method comprises detecting an adenine or guanine base at rs17237514 and an adenine or guanine base at rs4335725. When the haplotype comprises an adenine in the forward sequence of rs17237514 and an adenine in the forward sequence of rs4335725, the haplotype is a protective haplotype indicating that the subject is less likely to develop AMD than a person without this haplotype.

The presence of a protective and/or risk variant (and/or a protective and/or risk haplotype) can be determined by standard nucleic acid detection assays including, for example, conventional SNP detection assays, which may include, for example, amplification-based assays, probe hybridization assays, restriction fragment length polymorphism assays, and/or direct nucleic acid sequencing. Exemplary protocols for preparing and analyzing samples of interest are discussed in the following sections.

### A. Preparation of Samples for Analysis

Polymorphisms can be detected in a target nucleic acid sample from an individual under investigation. In general, genomic DNA can be analyzed, which can be selected from any biological sample that contains genomic DNA or RNA. For example, genomic DNA can be obtained from peripheral blood leukocytes using standard approaches (QIAamp DNA Blood Maxi kit, Qiagen, Valencia, Calif.). Nucleic acids can be harvested from other samples, for example, cells in saliva, cheek scrapings, amniotic fluid, placental tissue, urine, hair, skin, blood, biopsies of the retina, kidney, or liver or other organs or tissues. Methods for purifying nucleic acids from biological samples suitable for use in diagnostic or other assays are known in the art.

Alternatively, an individual's genetic profile may be analyzed by inspecting a data set indicative of genetic characteristics previously derived from analysis of the individual's genome. A data set indicative of an individual's genetic characteristics may include a complete or partial sequence of the individual's genomic DNA, or a SNP map. Inspection of the data set including all or part of the individual's genome may optimally be performed by computer inspection. Screening may further comprise the step of producing a report identifying the individual and the identity of alleles at the site of at least one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809 SNPs, and/or proxy polymorphic sites.

### B. Detection of Polymorphisms in Target Nucleic Acids

The identity of bases present at the polymorphic sites ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809, can be determined in an individual using any of several methods known in the art. The polymorphisms can be detected by direct sequencing, amplification-based assays, probe hybridization-based assays, restriction fragment length polymorphism assays, denaturing gradient gel electrophoresis, single-strand conformation polymorphism analyses, and denaturing high performance liquid chromatography. Other methods to detect nucleic acid polymorphisms include the use of: Molecular Beacons (see, e.g., Piatek et al. (1998) NAT. BIOTECHNOL. 16:359-63; Tyagi and Kramer (1996) NAT. BIOTECHNOL. 14:303-308; and Tyagi et al. (1998) NAT. BIOTECHNOL. 16:49-53), the Invader assay (see, e.g., Neri et al. (2000) ADV. NUCL. ACID PROTEIN ANALYSIS 3826: 117-125 and U.S. Pat. No. 6,706,471), and the Scorpion assay (Thelwell et al. (2000) NUCL. ACIDS RES. 28:3752-3761 and Solinas et al. (2001) NUCL. ACIDS RES. 29:20).

The design and use of allele-specific probes for analyzing polymorphisms are described, for example, in EP 235,726, and WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes are designed to hybridize to a segment of target DNA such that the polymorphic site aligns with a central position of the probe.

Probe-based genotyping can be carried out using a “TaqMan” or “5′-nuclease assay,” as described in U.S. Pat. Nos. 5,210,015; 5,487,972; and 5,804,375; and Holland et al. (1988) PROC. NATL. ACAD. SCI. USA 88:7276-7280, each incorporated herein by reference. Examples of other techniques that can be used for polymorphic site genotyping include, but are not limited to, Amplifluor, Dye Binding-Intercalation, Fluorescence Resonance Energy Transfer (FRET), Hybridization Signal Amplification Method (HSAM), HYB Probes, Invader/Cleavase Technology (Invader/CFLP), Molecular Beacons, Origen, DNA-Based Ramification Amplification (RAM), rolling circle amplification, Scorpions, Strand displacement amplification (SDA), oligonucleotide ligation (Nickerson et al. (1990) PROC. NATL ACAD. SCI. USA 87:8923-8927) and/or enzymatic cleavage. Popular high-throughput polymorphic variant detection (e.g., SNP variant detection) methods also include template-directed dye-terminator incorporation (TDI) assay (Chen and Kwok (1997) NUCL. ACIDS RES. 25:347-353), the 5′-nuclease allele-specific hybridization TaqMan assay (Livak et al. (1995) NATURE GENET. 9:341-342), and the allele-specific molecular beacon assay (Tyagi et al. (1998) NATURE BIOTECH. 16:49-53).

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference. In some embodiments multiple assays are conducted using a microfluidic format. (See, e.g., Unger et al. (2000) SCIENCE 288:113-6.)

The design and use of allele-specific primers for analyzing polymorphisms are described, for example, in WO 93/22456. Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works particularly well when the polymorphic site is at the extreme 3′-end of the primer, because this position is most destabilizing to elongation from the primer.

The primers, once selected, can be used in standard PCR protocols in conjunction with another common primer that hybridizes to the upstream non-coding strand of the ROBO1 gene at a specified location upstream from the polymorphism. The common primers are chosen such that the resulting PCR products can vary from about 100 to about 300 bases in length, or about 150 to about 250 bases in length, although smaller (about 50 to about 100 bases in length) or larger (about 300 to about 500 bases in length) PCR products are possible. The length of the primers can vary from about 10 to 30 bases in length, or about 15 to 25 bases in length.

Primers or probes can be labeled by incorporating a label detectable by spectroscopic, photochemical, biochemical, immunochemical, radiological, radiochemical or chemical means. Useful labels include ³²P, fluorescent dyes, electron-dense reagents, enzymes, biotin, or haptens and proteins for which antisera or monoclonal antibodies are available.

Many of the methods for detecting polymorphisms involve amplifying DNA or RNA from target samples (e.g., amplifying the segments of the ROBO1 gene of an individual using ROBO1-specific primers) and analyzing the amplified gene segments. This can be accomplished by standard polymerase chain reaction (PCR and RT-PCR) protocols or other methods known in the art, and described in U.S. Pat. Nos. 4,683,195; 4,683,202; and 4,965,188; each incorporated herein by reference. Other suitable amplification methods include the ligase chain reaction (Wu and Wallace (1988) GENOMICS 4:560-569); the strand displacement assay (Walker et al. (1992) PROC. NATL. ACAD. SCI. USA 89:392-396, Walker et al. (1992) NUCL. ACIDS RES. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al. (1989) PROC. NATL. ACAD. SCI. USA 86:1173-1177); and self-sustained sequence replication (3SR) (Guatelli et al. (1990) PROC. NATL. ACAD. SCI. USA 87:1874-1878 and WO 92/08800); each incorporated herein by reference. Alternatively, methods that amplify the probe to detectable levels can be used, such as QB-replicase amplification (Kramer et al. (1989) NATURE, 339:401-402, and Lomeli et al. (1989) CLIN. CHEM. 35:1826-1831, both of which are incorporated herein by reference). A review of known amplification methods is provided in Abramson et al. (1993) CURRENT OPINION IN BIOTECHNOLOGY 4:41-47, incorporated herein by reference.

Amplification products generated using any of the above methods can be analyzed by the use of denaturing gradient gel electrophoresis. Different alleles can be identified based on sequence-dependent melting properties and electrophoretic migration in solution. See Erlich, ed., PCR Technology, Principles and Applications for DNA Amplification, Chapter 7 (W.H. Freeman and Co, New York, 1992). Upon generation of an amplified product, polymorphisms of interest can be identified by DNA sequencing methods, such as the chain termination method (Sanger et al. (1977) PROC. NATL. ACAD. SCI. 74:5463-5467) or PCR-based sequencing. See Sambrook et al., MOLECULAR CLONING, A LABORATORY MANUAL (2nd Ed., CSHP, New York 1989) and Zyskind et al., RECOMBINANT DNA LABORATORY MANUAL (Acad. Press, 1988).

Other useful analytical techniques that can detect the presence of a polymorphism in the amplified product include single-strand conformation polymorphism (SSCP) analysis, denaturing gradient gel electropohoresis (DGGE) analysis, and/or denaturing high performance liquid chromatography (DHPLC) analysis. In such techniques, different alleles can be identified based on sequence- and structure-dependent electrophoretic migration of single stranded PCR products. Amplified PCR products can be generated according to standard protocols, and heated or otherwise denatured to form single stranded products, which may refold or form secondary structures that are partially dependent on base sequence. An alternative method, referred to herein as a kinetic-PCR method, in which the generation of amplified nucleic acid is detected by monitoring the increase in the total amount of double-stranded DNA in the reaction mixture, is described in Higuchi et al. (1992) BIO/TECHNOLOGY, 10:413-417, incorporated herein by reference.

Polymorphic variant detection can also be accomplished by direct PCR amplification, for example, via Allele-Specific PCR (AS-PCR), which is the selective PCR amplification of one of the alleles to detect a polymorphic variant (e.g., a SNP variant). Selective amplification is usually achieved by designing a primer such that the primer will match/mismatch one of the alleles at the 3′-end of the primer. The amplifying may result in the generation of ROBO1 allele-specific oligonucleotides, which span any of the SNPs, including, for example, ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809. The ROBO1-specific primer sequences and ROBO1 allele-specific oligonucleotides may be derived from the coding (exons) or non-coding (promoter, 5′ untranslated, introns or 3′ untranslated) regions of the ROBO1 gene. Polymorphic variant detection also can be accomplished using restriction fragment length polymorphism (RFLP) analysis, where the presence or absence of a particular variant at a polymorphic site creates or eliminates a restriction site for a particular endonuclease, creating a different pattern of fragment lengths, depending upon the variant present, when nucleic acid containing the polymorphic variant is exposed to the endonuclease.

A wide variety of other methods are known in the art for detecting polymorphisms in a biological sample. See, e.g., U.S. Pat. No. 6,632,606; Shi (2002) AM. J. PHARMACOGENOMICS 2:197-205; Kwok et al. (2003) CURR. ISSUES BIOL. 5:43-60). Detection of the single nucleotide polymorphic form (i.e., the presence or absence of the variant at ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809), alone and/or in combination with each other and/or in combination with additional ROBO1 gene polymorphisms, may increase the probability of an accurate diagnosis.

In one embodiment, screening involves determining the presence or absence of the variant at ROBO1_Ser162Ser. In another embodiment, screening involves determining the presence or absence of the variant at rs7615149. In another embodiment, screening involves determining the presence or absence of the variant at rs6548621. In another embodiment, screening involves determining the presence or absence of the variant at rs7629503. In another embodiment, screening involves determining the presence or absence of the variant at rs9309833. In another embodiment, screening involves determining the presence or absence of the variant at rs10865579. In another embodiment, screening involves determining the presence or absence of the variant at rs1393370. In another embodiment, screening involves determining the presence or absence of the variant at rs3923526. In another embodiment, screening involves determining the presence or absence of the variant at rs59931439. In another embodiment, screening involves determining the presence or absence of the variant at rs7640053. In another embodiment, screening involves determining the presence or absence of the variant at rs13090440. In another embodiment, screening involves determining the presence or absence of the variant at rs4680962. In another embodiment, screening involves determining the presence or absence of the variant at rs4510348. In another embodiment, screening involves determining the presence or absence of the variant at rs9810404. In another embodiment, screening involves determining the presence or absence of the variant at rs4513416. In another embodiment, screening involves determining the presence or absence of the variant at rs7624099. In another embodiment, screening involves determining the presence or absence of the variant at rs9853257. In another embodiment, screening involves determining the presence or absence of the variant at rs4284943. In another embodiment, screening involves determining the presence or absence of the variant at rs13058752. In another embodiment, screening involves determining the presence or absence of the variant at rs13076006. In another embodiment, screening involves determining the presence or absence of the variant at rs4680960. In another embodiment, screening involves determining the presence or absence of the variant at rs1546037. In another embodiment, screening involves determining the presence or absence of the variant at rs1387665. In another embodiment, screening involves determining the presence or absence of the variant at rs6548625. In another embodiment, screening involves determining the presence or absence of the variant at rs7637338. In another embodiment, screening involves determining the presence or absence of the variant at rs4279056. In another embodiment, screening involves determining the presence or absence of the variant at rs9871445. In another embodiment, screening involves determining the presence or absence of the variant at rs9826366. In another embodiment, screening involves determining the presence or absence of the variant at rs9848827. In another embodiment, screening involves determining the presence or absence of the variant at rs9832405. In another embodiment, screening involves determining the presence or absence of the variant at rs723766. In another embodiment, screening involves determining the presence or absence of the variant at rs9873952. In another embodiment, screening involves determining the presence or absence of the variant at rs7626242. In another embodiment, screening involves determining the presence or absence of the variant at rs7622444. In another embodiment, screening involves determining the presence or absence of the variant at rs7622888. In another embodiment, screening involves determining the presence or absence of the variant at rs4264688. In another embodiment, screening involves determining the presence or absence of the variant at rs7623809.

The analysis of ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809 may be combined with each other and/or may be combined with analysis of polymorphisms in other genes associated with AMD, detection of protein markers of AMD (see, e.g., U.S. Patent Application Publication Nos. US2003/0017501 and US2002/0102581 and International Patent Application Publication Nos. WO0184149 and WO0106262), assessment of other risk factors of AMD (such as family history), with ophthalmological examination, and with other assays and procedures.

Screening also can involve detecting a haplotype which includes two or more polymorphic variants. In an exemplary embodiment, a haplotype is defined by the alleles present at rs6548621 and rs7615149. If the subject has the protective variant (a guanine) at rs7615149 and a thymine or cytosine at rs6548621, then the subject has a reduced risk of developing AMD (e.g., neovascular AMD) relative to the person without the haplotype. Additional polymorphic variants that may be included in a haplotype include those described herein and/or additional ROBO1 gene polymorphisms, and/or other genes associated with AMD and/or other risk factors. The polymorphic variants include, but are not limited to, those at ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809.

For the two or more polymorphic variants, one determines if the risk variant is present or absent (for risk variant polymorphic variants) and/or if the common allele is present or absent (for protective variants) in order to diagnose a subject for being at increased risk of developing AMD. Conversely, for the two or more polymorphic variants, one can determine if the common allele is present or absent (for risk variants) and/or the protective variant is present or absent (for protective variants) in order to diagnose a subject for being at reduced risk of developing AMD.

A polymorphic variant (e.g., a SNP variant) either individually or within a genetic profile for AMD as described herein (e.g., ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809) may be detected directly or indirectly. Direct detection refers to determining the presence or absence of a specific polymorphic variant identified in the genetic profile using a suitable nucleic acid, such as an oligonucleotide in the form of a probe or primer as described above. Alternatively, direct detection can include querying a pre-produced database comprising all or part of the individual's genome for a specific polymorphic variant in the genetic profile. Other direct methods are described herein and are known to those skilled in the art. Indirect detection refers to determining the presence or absence of a specific polymorphic variant identified in the genetic profile by detecting a surrogate or proxy polymorphic variant that is in linkage disequilibrium with the polymorphic variant in the individual's genetic profile. Detection of a proxy polymorphic variant is indicative of a polymorphic variant of interest and is increasingly informative to the extent that the polymorphic variants are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

It is also understood that a genetic profile as described herein may include one or more nucleotide polymorphism(s) that are in linkage disequilibrium with a polymorphism that is causative of disease. In this case, the polymorphic variant in the genetic profile is a surrogate polymorphic variant for the causative polymorphism.

Genetically linked polymorphic variants, including surrogate or proxy polymorphic variants, can be identified by methods known in the art. Non-random associations between polymorphisms (including single nucleotide polymorphisms, or SNPs) at two or more loci are measured by the degree of linkage disequilibrium (LD). The degree of linkage disequilibrium is influenced by a number of factors including genetic linkage, the rate of recombination, the rate of mutation, random drift, non-random mating and population structure. Moreover, loci that are in LD do not have to be located on the same chromosome, although most typically they occur as clusters of adjacent variations within a restricted segment of DNA. Polymorphisms that are in complete or close LD with a particular disease-associated polymorphic variant are also useful for screening, diagnosis, and the like.

### C. Protein-Based or Phenotypic Detection of Polymorphism

Where polymorphisms are associated with a particular phenotype, then individuals that contain the polymorphism can be identified by checking for the associated phenotype. For example, where a polymorphism causes an alteration in the structure, sequence, expression and/or amount of a protein or gene product, and/or size of a protein or gene product, the polymorphism can be detected by protein-based assay methods.

Protein-based assay methods include electrophoresis (including capillary electrophoresis and one- and two-dimensional electrophoresis), chromatographic methods such as high performance liquid chromatography (HPLC), thin layer chromatography (TLC), hyperdiffusion chromatography, and mass spectrometry.

Where the structure and/or sequence of a protein is changed by a polymorphism of interest, one or more antibodies that selectively bind to the altered form of the protein can be used. Such antibodies can be generated and employed in detection assays such as fluid or gel precipitin reactions, immunodiffusion (single or double), immunoelectrophoresis, radioimmunoassay (RIA), enzyme-linked immunosorbent assays (ELISAs), immunofluorescent assays, Western blotting and others.

### III. KITS

In certain embodiments, one or more oligonucleotides are provided in a kit or on device (e.g., an array) useful for detecting the presence of a predisposing or a protective polymorphism in a nucleic acid sample of an individual whose risk for AMD is being assessed. A useful kit can contain oligonucleotides specific for particular alleles of interest as well as instructions for their use to determine risk for AMD. In some cases, the oligonucleotides may be in a form suitable for use as a probe, for example, fixed to an appropriate support membrane. In other cases, the oligonucleotides can be intended for use as amplification primers for amplifying regions of the loci encompassing the polymorphic sites, as such primers are useful in a preferred embodiment. Alternatively, useful kits can contain a set of primers comprising an allele-specific primer for the specific amplification of alleles. As yet another alternative, a useful kit can contain antibodies to a protein that is altered in expression levels, structure and/or sequence when a polymorphism of interest is present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

In one embodiment, a kit or device for diagnosing susceptibility to age-related macular degeneration (AMD) in a subject comprising oligonucleotides that distinguish alleles at at least one polymorphic site in the ROBO1 gene associated with risk of developing AMD. The oligonucleotides may distinguish alleles at at least one polymorphic site selected from the group consisting of ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809. In an exemplary embodiment, the oligonucleotides are primers for nucleic acid amplification of a region spanning a ROBO1 gene polymorphic site selected from the group consisting of ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809. In another exemplary embodiment, the oligonucleotides are probes for nucleic acid hybridization of a region spanning a ROBO1 gene polymorphic site selected from the group consisting of ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and rs7623809.

In certain embodiments, a kit or device may include oligonucleotides that distinguish alleles at more than one polymorphic site in the ROBO1 gene. For example the kit or device may include oligonucleotides that distinguish alleles, for example, at rs6548621 and rs7615149.

In still other embodiment, a kit or device may include oligonucleotides that distinguish alleles at rs1061170 (CFH), rs800292 (CFH), rs10490924 (LOC387715), rs11200638 (ARMS2/HTRA1), rs2672598 (ARMS2/HTRA1), rs10664316 (ARMS2/HTRA1), rs1049331 (ARMS2/HTRA1), rs12900948 (RORA), rs4335725 (RORA), rs8034864 (RORA), and rs1045216 (PLEKHA1) or other alleles associated with AMD.

### V. ANALYSIS SYSTEMS AND REPORTS

In a further aspect, disclosed herein is a system for analyzing one or more SNPs selected from the group of ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 comprising: reagents to detect (directly or indirectly) in a sample from the patient the presence or absence of one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 SNPs (including the presence or absence of a specific variant at a particular SNP); hardware to perform detection of the SNPs; and a processor to execute stored instruction sequences (for example, software) that analyze the detected information (e.g., to identify and/or calculate a level of one or more SNPs), to determine if the patient is at risk of developing, or has, AMD, and/or to determine if the patient is responsive to a treatment. The reagents to detect one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 SNPs (including the presence or absence of a specific variant at a particular SNP) may be, for example, any of those described herein, including primers, probes, and other molecules that bind to and/or amplify one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 SNPs (including a specific variant at a particular SNP) and/or a proxy polymorphic site (including a proxy polymorphic variant). The hardware is preferably a machine or computer to perform the detection step, and the processor may be by, for example, part of a computer or machine specifically configured to perform the analysis described herein.

Suitable software and processors are well known in the art and are commercially available. The program may be embodied in software and stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

After detecting (including detecting the presence or absence of) one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 SNPs (including the presence or absence of a specific variant at a particular SNP), and producing the assay results, findings, diagnoses, predictions and/or treatment, they are typically recorded and/or communicated to, for example, medical professionals and/or patients. In certain embodiments, the assay results, findings, diagnoses, predictions and/or treatment recommendations are communicated to the patient, directly, or to the patient's treating physician, after the assay and analysis is completed. The assay results, findings, diagnoses, predictions and/or treatment recommendations may be communicated to medical professionals and/or patients by any means of communication, such as a written report (e.g., on paper), an auditory report, or an electronic record.

Communication may be facilitated by use electronic forms of communication and/or by use of a computer, such as in case of email or telephone communications. In certain embodiments, the communication containing assay results, findings, diagnoses, predictions and/or treatment recommendations may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments, all or some of the method steps, including the assaying of samples, diagnosing/prognosing of diseases, and communicating of assay results, findings, diagnoses, predictions and/or treatment recommendations, may be carried out in diverse (e.g., foreign) jurisdictions. For example, in some embodiments the assays are performed, or the assay results analyzed, in a country or jurisdiction which differs from the country or jurisdiction to which the assay results, findings, diagnoses, predictions and/or treatment recommendations are communicated.

To facilitate diagnosis, the presence, absence, and/or level of one or more of the ROBO1_Ser162Ser, rs7615149, rs6548621, rs7629503, rs9309833, rs10865579, rs1393370, rs3923526, rs59931439, rs7640053, rs13090440, rs4680962, rs4510348, rs9810404, rs4513416, rs7624099, rs9853257, rs4284943, rs13058752, rs13076006, rs4680960, rs1546037, rs1387665, rs6548625, rs7637338, rs4279056, rs9871445, rs9826366, rs9848827, rs9832405, rs723766, rs9873952, rs7626242, rs7622444, rs7622888, rs4264688, and/or rs7623809 SNPs (including the presence, absence, and/or level of a specific variant at a particular SNP) and/or of a proxy polymorphic site (including the presence, absence, and/or level of a proxy polymorphic variant) can be displayed on a display device or contained electronically or in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

The methods disclosed herein, when practiced for commercial diagnostic purposes, generally produce a report or summary of the presence or absence of one or more of the SNPs described herein (including the presence or absence of a specific variant at a particular SNP) and/or a proxy polymorphic site (including the presence or absence of a proxy polymorphic variant). The methods disclosed herein also can produce a report comprising one or more predictions and/or diagnoses concerning a patient, for example whether the patient is at risk of developing, or has, dry or neovascular AMD.

The methods and reports disclosed herein can further include storing the report in a database. Alternatively, the method can further create a record in a database for the subject and populate the record with data. Reports can include a paper report, an auditory report, or an electronic record. It is contemplated that the report is provided to a physician and/or the patient. The receiving of the report can further include establishing a network connection to a server computer that includes the data and report and requesting the data and report from the server computer. The methods provided herein may also be automated in whole or in part.

In another aspect, the methods disclosed herein provide an article of manufacture having a computer-readable medium with computer-readable instructions embodied thereon for performing the methods and implementing the systems described herein. In particular, the stored instruction sequences of the present disclosure may be embedded on a computer-readable medium, such as, but not limited to, a floppy disk, a hard disk, an optical disk, a magnetic tape, a PROM, an EPROM, CD-ROM, or DVD-ROM or downloaded from a server. The stored instruction sequences may be embedded on the computer-readable medium in any number of computer-readable instructions, or languages such as, for example, FORTRAN, PASCAL, C, C++, Java, C#, Tcl, BASIC and assembly language. Further, the computer-readable instructions may, for example, be written in a script, macro, or functionally embedded in commercially available software (such as, e.g., EXCEL or VISUAL BASIC).

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the method remains operable. Moreover, two or more steps or actions may be conducted simultaneously.

### IV. PROGNOSIS AND DIAGNOSIS OF AMD BY DETERMINING GENE EXPRESSION LEVELS

Also disclosed herein is a method of determining whether a subject (e.g., a human subject) is at risk of developing, or has, age-related macular degeneration (AMD), for example, dry AMD or neovascular (wet) AMD by determining (e.g., measuring) the gene expression of one or more genes associated with AMD as discussed below. The method includes the steps of: (a) measuring the amount of a ROBO1 gene product in a test sample harvested from the mammal; and (b) comparing the amount of the gene or gene product against a control value, wherein an amount of the gene or gene product in the sample greater than the control value is indicative that the mammal is at risk of developing, or has, AMD. The method may further comprise (c) measuring the amount of a RORA gene product in a test sample harvested from the mammal; and (d) comparing the amount of the gene or gene product against a control value, wherein an amount of the gene or gene product in the sample greater than the control value is indicative that the mammal is at risk of developing, or has, AMD.

RORA is understood to be a nuclear receptor involved in many pathophysiological processes such as cerebellar ataxia, inflammation, atherosclerosis and angiogenesis. (Chauvet et al. (2004) “The gene encoding human retinoic acid-receptor-related orphan receptor α is a target for hypoxia-inducible factor 1,” BIOCHEM J 384(1):79-85.) As used herein, the term “RORA gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the RORA gene as reported in the NCBI gene database under gene ID: 6095, gene location accession no. NC_{—}000015.8 (58576755..59308794, complement) or a strand complementary thereto; (ii) the full length sequence of the RORA gene reported in the NCBI gene database under gene ID: 6095, gene location accession no. NC_{—}000015.8 (58576755..59308794, complement); (iii) a naturally occurring allelic variant of one of the foregoing sequences; or (iv) a nucleic acid sequence complementary to one of the foregoing sequences.

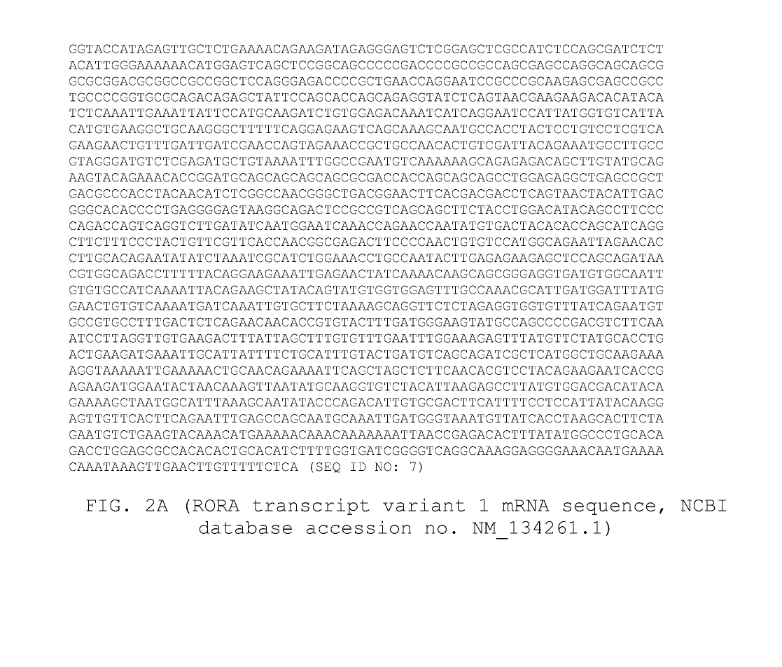

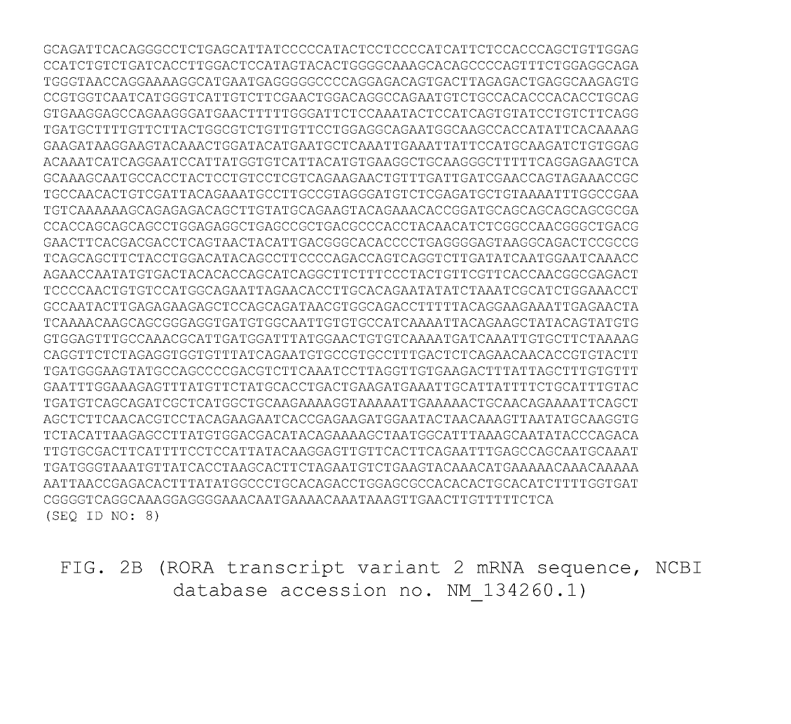

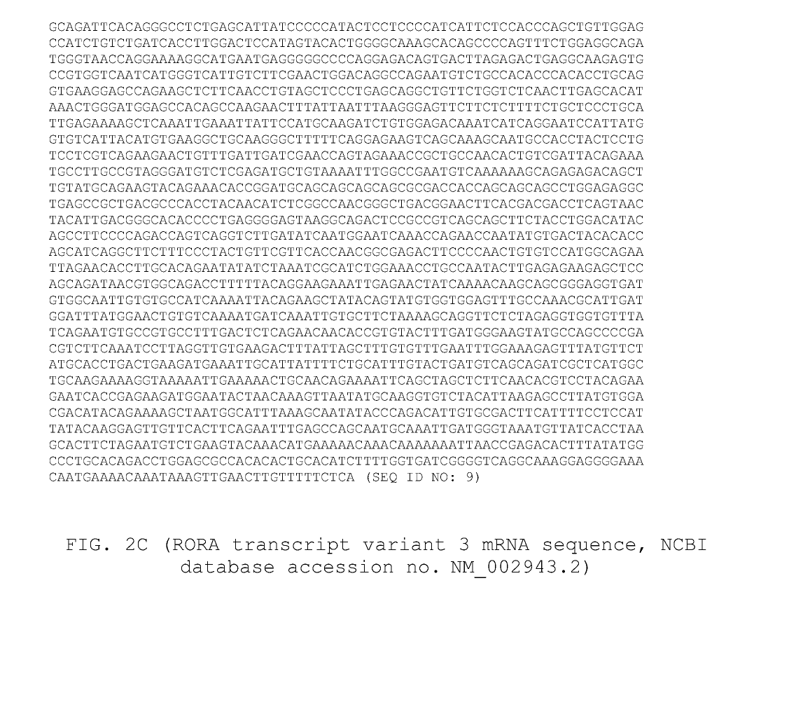

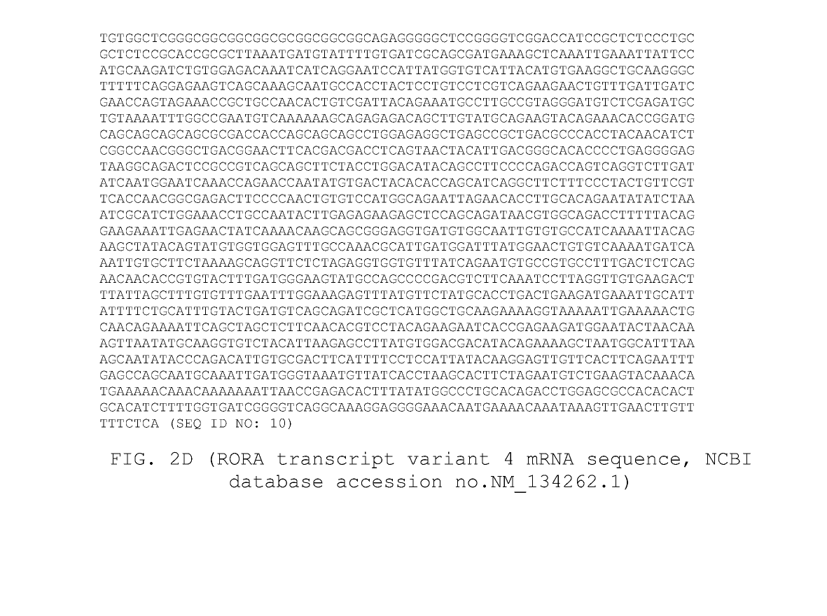

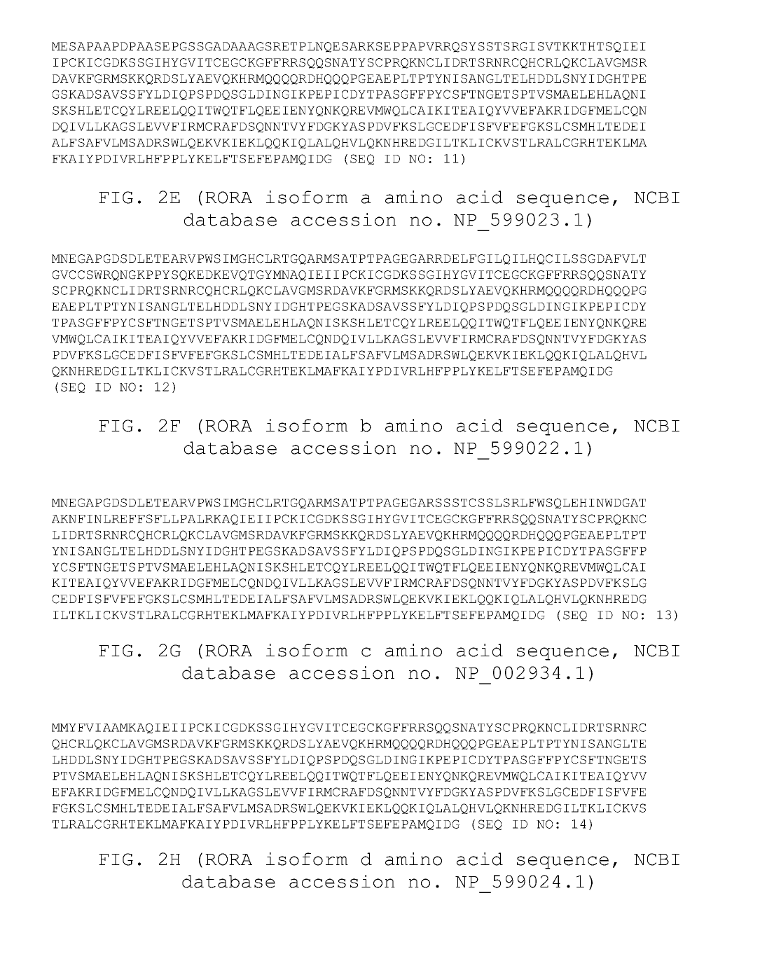

As used herein, a “RORA gene product” is understood to mean (i) a nucleic acid, for example, a sequence at least 75, at least 150, or at least 225 nucleotides in length that hybridizes under specific hybridization and washing conditions to the RORA gene (either the sense or anti-sense sequence); (ii) a nucleic acid sequence that is at least 90%, more preferably at least 95%, and more preferably at least 98% identical to the mRNA sequence shown in one of FIGS. 2A-D, or a nucleic acid sequence that hybridizes under specific hybridization and washing conditions to the sequence shown in one of FIGS. 2A-D; or (iii) a peptide or protein at least 25, at least 50, or at least 75 amino acids in length that is at least 95%, more preferably at least 98%, and more preferably at least 99% identical to the amino acid sequence shown in one of FIGS. 2E-H.

The nucleic acid encoding human RORA gene spans approximately 732 kb in length as reported in the NCBI gene database under gene ID: 6095, gene location accession no. NC_{—}000015.8 (58576755..59308794, complement). The RORA gene has been reported to generate four splicing transcript variants. The transcript variant 1 comprises eleven exons as reported in the NCBI nucleotide database under accession no. NM_{—}134261; the protein encoded by transcript variant 1 is 523 amino acids in length as reported in the NCBI protein database under accession no. NP_{—}599023. The transcript variant 2 comprises twelve exons as reported in the NCBI nucleotide database under accession no. NM_{—}134260; the protein encoded by transcript variant 2 is 556 amino acids in length as reported in the NCBI protein database under accession no. NP_{—}599022. Transcript variant 3 comprises eleven exons as reported in the NCBI nucleotide database under accession no. NM_{—}002943; the protein encoded by transcript variant 3 is 548 amino acids in length as reported in the NCBI protein database under accession no. NP_{—}002934. Transcript variant 4 comprises ten exons as reported in the NCBI nucleotide database under accession no. NM_{—}134262; the protein encoded by transcript variant 4 is 468 amino acids in length as reported in the NCBI protein database under accession no. NP_{—}599024.

It is understood that the RORA gene may have more transcript variants. For example, it has been suggested that the RORA gene may generate at least fifteen transcript variants (see the ECGENE database, available at the web site, genome.ewha.ac.kr/ECgene/, under entry H15C5901). Polymorphisms have also been identified in the coding regions and untranslated regions of the exons, as well as in the introns and in the chromosome outside of the transcript region or regions of the RORA gene. As examples of the polymorphisms in the RORA gene, the NCBI SNP database reports 5,746 specific polymorphic sites for the RORA gene under gene ID: 6095. The mRNA sequences and the amino acid sequences of RORA are set forth in FIGS. 2A-D and in FIGS. 2E-H, respectively.

In certain embodiments, additional gene products may also be measured from the following genes: CREB5 (reported in the NCBI gene database under gene ID: 9586, gene location accession no. NC_{—}000007.13 (28338940..28865511)), CXCL13 (reported in the NCBI gene database under gene ID: 10563, gene location accession no. NC_{—}000004.10 (78651931..78752010)), ENPP2 (reported in the NCBI gene database under gene ID: 5168, gene location accession no. NC_{—}000008.9 (120638500..120720287, complement)), FAM169A (also known as KIAA0888, reported in the NCBI gene database under gene ID: 26049, gene location accession no. NC_{—}000005.8 (74109155..74198371, complement)), IGKV1-5 (reported in the NCBI gene database under gene ID: 28299, gene location accession no. NC_{—}000002.11 (89246819..89247294, complement)), IL1A (reported in the NCBI gene database under gene ID: 3552, gene location accession no. NC_{—}000002.10 (113247963..113259442, complement)), MMP7 (reported in the NCBI gene database under gene ID: 4316, gene location accession no. NC_{—}000011.8 (101896449..101906688, complement)), RGS13 (reported in the NCBI gene database under gene ID: 6003, gene location accession no. NC_{—}000001.9 (190871905..190896013)), RPS6KA2 (reported in the NCBI gene database under gene ID: 6196, gene location accession no. NC_{—}000006.10 (166742844..167195761, complement)), UGT2B17 (reported in the NCBI gene database under gene ID: 7367, gene location accession no. NC_{—}000004.11 (69402902..69434245, complement)), CRIM1 (reported in the NCBI gene database under gene ID: 51232, gene location accession no. NC_{—}000002.10 (36436901..36631782) (available at the web site, www.ncbi.nlm.nih.gov)), CXCR4 (reported in the NCBI gene database under gene ID: 7852, gene location accession no. NC_{—}000002.10 (136588389..136592195, complement)), C5orf26 (reported in the NCBI gene database under gene ID: 114915, gene location accession no. NC_{—}000005.8 (111524125..111524816)), IGHG3 (reported in the NCBI gene database under gene ID: 3502, gene location accession no. NC_{—}000014.7 (105303296..105308787, complement)), IGLJ3 (reported in the NCBI gene database under gene ID: 28831, gene location accession no. NC_{—}000022.9 (21577168..21577205)), SHQ1 (reported in the NCBI gene database under gene ID: 55164, gene location accession no. NC_{—}000003.10 (72881118..72980288, complement)), DNAJC6 (reported in the NCBI gene database under gene ID: 9829, gene location accession no. NC_{—}000001.9 (65503018..65654140)), C6orf105 (reported in the NCBI gene database under gene ID: 84830, gene location accession no. NC_{—}000006.10 (11821895..11887052, complement)), NALP1 (reported in the NCBI gene database under gene ID: 22861, gene location accession no. NC_{—}0000017.9 (5345443..5428556, complement)), IGHM ((reported in the NCBI gene database under gene ID: 3507, gene location accession no. NC_{—}000014.8 (106318037..106322322, complement)), NLRP2 (also known as NALP2, reported in the NCBI gene database under gene ID: 55655, gene location accession no. NC_{—}000019.8 (60169579..60204318)), PKP2 (reported in the NCBI gene database under gene ID: 5318, gene location accession no. NC_{—}000012.10 (32834947..32941047, complement)), PLA2G4A (reported in the NCBI gene database under gene ID: 5321, gene location accession no. NC_{—}000001.9 (185064655..185224736)), TANC1 (reported in the NCBI gene database under gene ID: 85461, gene location accession no. NC_{—}000002.10 (159533392..159797416)), UCHL1 (reported in the NCBI gene database under gene ID: 7345, gene location accession no. NC_{—}000004.10 (40953686..40965203)), ABCA1 (reported in the NCBI gene database under gene ID: 19, gene location accession no. NC_{—}000009.10 (106583104..106730257, complement)), VCAN (reported in the NCBI gene database under gene ID: 1462, gene location accession no. NC_{—}000005.8 (82803339..82912737)), and/or FAM38B (reported in the NCBI gene database under gene ID: 63895, gene location accession no. NC_{—}000018.8 (10660850..10687814, complement)).

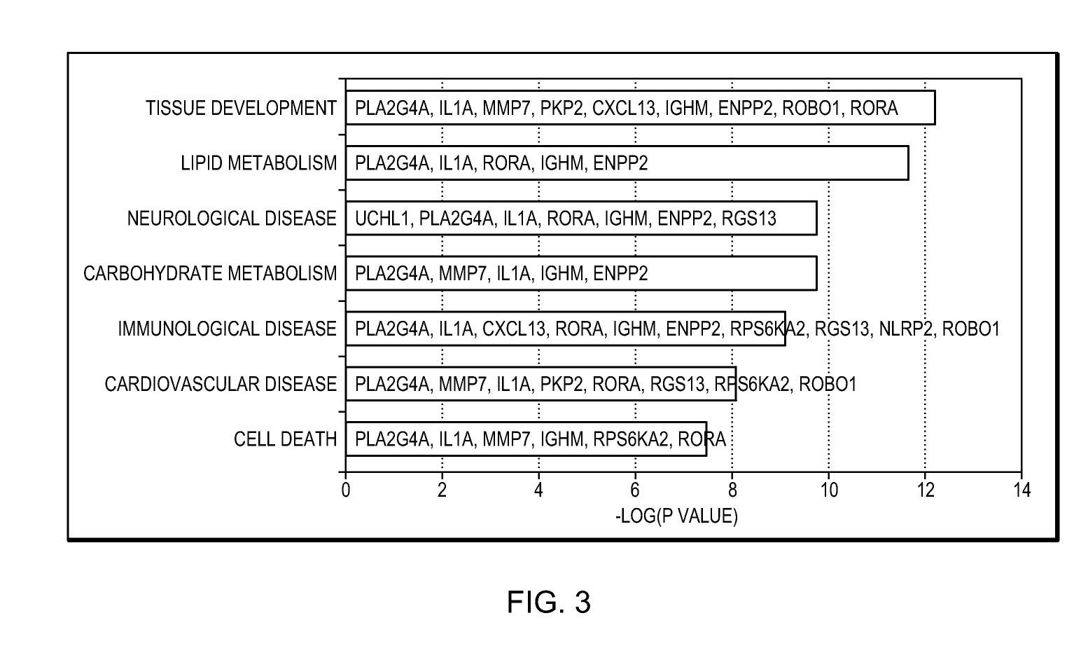

For example, but without limitation, one or more gene products to be measured can be selected according to those grouped in a particular network, as shown in Table 1, or according to those grouped by a particular biological function, as shown in Table 2 or in FIG. 3. Moreover, any of the molecules shown in Table 1 can be used in combination as groups of markers. It should be understood that any one or more of the upregulated markers can be combined with any one or more downregulated marker, as well.

TABLE 1

| | | | Focus | |
|---|---|---|---|---|
| Network | Molecules in Network | Score | Molecules | functions |
| 1 | ABCA1, cholesterol sulfate, CXCL13, | 33 | 12 | Tissue Morphology, |
| | CXCR4, DEFB104A, DEFB4 (includes | | | Dermatological Diseases |
| | EG: 56519), DOK5, ERK, FCGR1B, | | | and Conditions, Organ |
| | FCGR1C, IGHG3, IL1, IL1/IL6/TNF, | | | Morphology |
| | IL1A, IL1F5, IL1F6, IL1F7, IL1F8, | | | |
| | IL1F9, IL1F10, LDL, Mapk, MMP7, | | | |
| | NFkB (complex), NALP2, P38 MAPK, | | | |
| | PELI2, PLA2G4A, RGS13, RORA, RPS6KA2, | | | |
| | S100A3, Tgf beta, TRIB1, VCAN | | | |
| 2 | ALDH1A1, COL4A1, CRIM1, DSP, | 8 | 4 | Protein Synthesis, |
| | EEF1D, EIF3C, EIF4A1, EIF5A, | | | Drug Metabolism, |
| | ELAVL2, ENPP2, IGFBP7, KRT5, | | | Lipid Metabolism |
| | MYCN, NMI, PKP2, retinoic acid, | | | |
| | RPL3, RPL4, RPL6, RPL11, RPL29, | | | |
| | RPL23A (includes EG: 6147), RPS3, | | | |
| | RPS16, RPS19, RPS20, RPS4X, | | | |
| | SLC38A2, TPI1, UCHL1, USP3, | | | |
| | ZBTB17, ZEB2, ZFAND5, ZNF217 | | | |
| 3 | APOA1, FAM169A | 3 | 1 | Antigen Presentation, |
| | | | | Carbohydrate Metabolism, |
| | | | | Cardiovascular Disease |
| 4 | MIRN93 (includes EG: 407050), TANC1 | 3 | 1 | Cancer, Reproductive |
| | | | | System Disease |
| 5 | DNAJC, DNAJC6,Hsp22/Hsp40/Hsp90, MIRN128-1 | 2 | 1 | |
| | (includes EG: 406915), MIRN128-2 | | | |
| | (includes EG: 406916) | | | |
| 6 | FAM38B, MIRN34C (includes EG: 407042), | 2 | 1 | Cancer, Gastrointestinal |
| | MIRN98 (includes EG: 407054), MIRNLET7A1, | | | Disease, Hepatic System |
| | MIRNLET7A2, MIRNLET7A3, MIRNLET7B | | | Disease |
| | (includes EG: 406884), MIRNLET7C, | | | |
| | MIRNLET7F1 (includes EG: 406888), | | | |
| | MIRNLET7F2 (includes EG: 406889), | | | |
| | MIRNLET7G (includes EG: 406890) | | | |

TABLE 2

| Biological Function | P-Value | Molecules |
|---|---|---|
| Genetic Disorder | 4.29 × 10⁻⁶-3.59 × 10⁻² | IL1A, MMP7, PKP2, CXCR4, VCAN, |
| | | ABCA1, UCHL1, PLA2G4A, IGHG3, |
| | | CXCL13, RORA, ENPP2, RGS13, NALP2, |
| | | CRIM1 |
| Tissue Development | 4.52 × 10⁻⁶-3.61 × 10⁻² | PLA2G4A, IL1A, PKP2, CXCL13, CXCR4, |
| | | ENPP2, VCAN |
| Cellular Function and | 9.04 × 10⁻⁶-1.76 × 10⁻² | IL1A, CXCL13, CXCR4, ABCA1 |
| Maintenance | | |
| Cellular Movement | 9.04 × 10⁻⁶-3.98 × 10⁻² | PLA2G4A, IL1A, MMP7, CXCL13, CXCR4, |
| | | ENPP2, VCAN |
| Hematological System | 9.04 × 10⁻⁶-3.86 × 10⁻² | PLA2G4A, IL1A, CXCL13, RORA, CXCR4, |
| Development and | | ABCA1 |
| Function | | |
| Humoral Immune | 9.04 × 10⁻⁶-3.86 × 10⁻² | PLA2G4A, IL1A, MMP7, IGHG3, CXCL13, |
| Response | | RORA, CXCR4 |
| Lipid Metabolism | 1.32 × 10⁻⁵-3.98 × 10⁻² | PLA2G4A, MMP7, IL1A, RORA, ENPP2, |
| | | ABCA1 |
| Molecular Transport | 1.32 × 10⁻⁵-3.98 × 10⁻² | PLA2G4A, MMP7, IL1A, CXCL13, RORA, |
| | | CXCR4, ENPP2, ABCA1 |
| Small Molecule | 1.32 × 10⁻⁵-3.98 × 10⁻² | PLA2G4A, IL1A, MMP7, RORA, ENPP2, |
| Biochemistry | | RGS13, VCAN, ABCA1 |
| Carbohydrate Metabolism | 5.4 × 10⁻³-3.36 × 10⁻² | PLA2G4A, MMP7, IL1A, ENPP2, ABCA1 |
| Respiratory System | 5.4 × 10⁻⁵-3.79 × 10⁻³ | PLA2G4A, IL1A, ABCA1 |
| Development and | | |
| Function | | |
| Tissue Morphology | 5.4 × 10⁻⁵-3.86 × 10⁻² | PLA2G4A, MMP7, IL1A, CXCL13, CXCR4, |
| | | ABCA1 |
| Hematological Disease | 7.53 × 10⁻⁵-3.86 × 10⁻² | PLA2G4A, MMP7, IL1A, PKP2, CXCL13, |
| | | CXCR4, RORA, ABCA1 |
| Skeletal and Muscular | 1.17 × 10⁻⁴-3 × 10⁻² | PLA2G4A, IL1A, CXCL13, CXCR4, |
| Disorders | | RPS6KA2 |
| Immunological Disease | 1.25 × 10⁻⁴-3.12 × 10⁻² | PLA2G4A, IL1A, CXCL13, RORA, CXCR4, |
| | | RGS13, NALP2, ABCA1 |
| Reproductive System | 1.42 × 10⁻⁴-3 × 10⁻² | UCHL1, PLA2G4A, IL1A, MMP7, CXCL13, |
| Disease | | CXCR4, CRIM1, VCAN |
| Cancer | 2.83 × 10⁻⁴-3.67 × 10⁻² | PLA2G4A, MMP7, IL1A, IGHG3, CXCL13, |
| | | CXCR4, ENPP2, CRIM1, VCAN |
| Cell-To-Cell Signaling | 2.83 × 10⁻⁴-3.98 × 10⁻² | UCHL1, IL1A, MMP7, CXCL13, PKP2, |
| and Interaction | | CXCR4, VCAN, ABCA1 |
| Cellular Growth and | 3.56 × 10⁻⁴-3 × 10⁻² | UCHL1, PLA2G4A, MMP7, IL1A, CXCR4, |
| Proliferation | | ENPP2, VCAN |
| Cardiovascular Disease | 4.76 × 10⁻⁴-3.49 × 10⁻² | PLA2G4A, MMP7, IL1A, PKP2, CXCR4, |
| | | ABCA1 |
| Metabolic Disease | 4.82 × 10⁻⁴-1.13 × 10⁻² | IL1A, RORA, ABCA1 |
| Cell Death | 6.87 × 10⁻⁴-3 × 10⁻² | PLA2G4A, MMP7, IL1A, CXCR4, |
| | | RPS6KA2, VCAN |
| Connective Tissue | 6.87 × 10⁻⁴-3 × 10⁻² | PLA2G4A, MMP7, IL1A, CXCL13, CXCR4, |
| Disorders | | ENPP2, RPS6KA2 |
| Inflammatory Disease | 9.27 × 10⁻⁴-3 × 10⁻² | PLA2G4A, MMP7, IL1A, CXCL13, CXCR4, |
| | | ABCA1 |
| Cardiovascular System | 9.79 × 10⁻⁴-3.98 × 10⁻² | PLA2G4A, IL1A, CXCL13, PKP2, CXCR4, |
| Development and | | ENPP2, VCAN |
| Function | | |
| Cell Morphology | 9.79 × 10⁻⁴-3.86 × 10⁻² | PLA2G4A, IL1A, CXCR4 |
| Cellular Development | 9.79 × 10⁻⁴-3.86 × 10⁻² | IL1A, RORA, CXCR4, RPS6KA2, VCAN |
| Dermatological Diseases | 9.99 × 10⁻⁴-3 × 10⁻² | IL1A, CXCL13, CXCR4, RGS13 |
| and Conditions | | |
| Skeletal and Muscular | 1.03 × 10⁻³-3.98 × 10⁻² | PLA2G4A, MMP7, IL1A, PKP2, CXCR4, |
| System Development and | | ENPP2, RGS13 |
| Function | | |
| Tumor Morphology | 1.03 × 10⁻³-3 × 10⁻² | IL1A, MMP7, CXCR4, ENPP2 |
| Drug Metabolism | 1.14 × 10⁻³-3.86 × 10⁻² | PLA2G4A, IL1A, ABCA1 |
| Gastrointestinal Disease | 1.14 × 10⁻³-2.02 × 10⁻² | PLA2G4A, IL1A, MMP7, IGHG3 |
| Cell-mediated Immune | 1.2 × 10⁻³-2.5 × 10⁻² | PLA2G4A, IL1A, MMP7, IGHG3, CXCL13, |
| Response | | RORA, CXCR4 |
| Hematopoiesis | 1.2 × 10⁻³-3 × 10⁻² | IL1A, MMP7, CXCL13, RORA, CXCR4 |
| Lymphoid Tissue | 1.2 × 10⁻³-3 × 10⁻² | IL1A, CXCL13, RORA, CXCR4 |
| Structure and | | |
| Development | | |
| Organismal Injury and | 1.2 × 10⁻³-3.86 × 10⁻² | PLA2G4A, MMP7, IL1A, PKP2, CXCR4, |
| Abnormalities | | ABCA1 |
| Nervous System | 1.26 × 10⁻³-2.87 × 10⁻² | UCHL1, IL1A, CXCR4, RORA |
| Development and | | |
| Function | | |
| Organ Development | 1.26 × 10⁻³-2.66 × 10⁻² | PLA2G4A, CXCL13, PKP2, RORA, CXCR4, |
| | | VCAN, ABCA1 |
| Cellular Assembly and | 1.27 × 10⁻³-3.86 × 10⁻² | UCHL1, PLA2G4A, IGHG3, CXCR4, |
| Organization | | ENPP2, VCAN, ABCA1 |
| Cellular Compromise | 1.27 × 10⁻³-3.12 × 10⁻² | CXCR4, RGS13, ABCA1 |
| Connective Tissue | 1.27 × 10⁻³-3.98 × 10⁻² | PLA2G4A, IL1A, CXCL13, ENPP2, VCAN |
| Development and | | |
| Function | | |
| Embryonic Development | 1.27 × 10⁻³-3.12 × 10⁻² | CXCR4, ENPP2, RPS6KA2, ABCA1 |
| Endocrine System | 1.27 × 10⁻³-1.51 × 10⁻² | IL1A, CXCR4 |
| Development and | | |
| Function | | |
| Endocrine System | 1.27 × 10⁻³-8.83 × 10⁻³ | MMP7, IL1A, CXCR4 |
| Disorders | | |
| Gene Expression | 1.27 × 10⁻³-4.04 × 10⁻² | PLA2G4A, IL1A, RORA |
| Hair and Skin | 1.27 × 10⁻³-3.12 × 10⁻² | IL1A, RORA, ABCA1 |
| Development and | | |
| Function | | |
| Immune Cell Trafficking | 1.27 × 10⁻³-2.26 × 10⁻² | PLA2G4A, MMP7, IL1A, CXCL13, CXCR4 |
| Inflammatory Response | 1.27 × 10⁻³-3.73 × 10⁻² | PLA2G4A, MMP7, IL1A, IGHG3, CXCL13, |
| | | CXCR4, ABCA1 |
| Ophthalmic Disease | 1.27 × 10⁻³-1.27 × 10⁻³ | VCAN |
| Organ Morphology | 1.27 × 10⁻³-1.89 × 10⁻² | PLA2G4A, IL1A, CXCL13, PKP2, RORA, |
| | | ABCA1 |
| Reproductive System | 1.27 × 10⁻³-2.75 × 10⁻² | PLA2G4A, CXCR4, ABCA1 |
| Development and | | |
| Function | | |
| Vitamin and Mineral | 1.27 × 10⁻³-1.83 × 10⁻² | CXCL13, CXCR4, ABCA1 |
| Metabolism | | |
| Respiratory Disease | 2 × 10⁻³-3.86 × 10⁻² | PLA2G4A, MMP7, ABCA1 |
| Cell Signaling | 2.23 × 10⁻³-3.98 × 10⁻² | IL1A, CXCL13, CXCR4, RORA, RGS13, |
| | | RPS6KA2, ABCA1 |
| Amino Acid Metabolism | 2.53 × 10⁻³-2.5 × 10⁻² | IL1A, VCAN |
| Cell Cycle | 2.53 × 10⁻³-5.06 × 10⁻³ | IL1A, RPS6KA2 |
| Developmental Disorder | 2.53 × 10⁻³-1.26 × 10⁻² | PLA2G4A, MMP7 |
| Infection Mechanism | 2.53 × 10⁻³-3 × 10⁻² | CXCR4 |
| Infectious Disease | 2.53 × 10⁻³-2.11 × 10⁻² | IL1A, CXCR4, CRIM1 |
| Neurological Disease | 2.53 × 10⁻³-1.26 × 10⁻² | UCHL1, PLA2G4A, IL1A, RORA, CXCR4, |
| | | ENPP2, CRIM1, VCAN, ABCA1 |
| Organismal Development | 2.53 × 10⁻³-4.1 × 10⁻² | PLA2G4A, IL1A |
| Renal and Urological | 2.53 × 10⁻³-3.79 × 10⁻³ | IL1A, ABCA1 |
| Disease | | |
| Antigen Presentation | 2.97 × 10⁻³-3.12 × 10⁻² | PLA2G4A, IL1A, MMP7, IGHG3, CXCL13, |
| | | CXCR4, ABCA1 |
| Hypersensitivity Response | 3.79 × 10⁻³-8.83 × 10⁻³ | IL1A |
| Nucleic Acid Metabolism | 5.06 × 10⁻³-3.98 × 10⁻² | RORA, RGS13, ABCA1 |
| Hepatic System | 6.32 × 10⁻³-6.32 × 10⁻³ | IL1A |
| Development and | | |
| Function | | |
| Hepatic System Disease | 7.57 × 10⁻³-1.26 × 10⁻² | IL1A, MMP7 |
| Organismal Functions | 7.57 × 10⁻³-7.57 × 10⁻³ | IL1A |
| Behavior | 1.01 × 10⁻²-3.61 × 10⁻² | UCHL1 |
| Protein Synthesis | 1.01 × 10⁻²-1.88 × 10⁻² | ABCA1 |
| Post-Translational | 1.38 × 10⁻²-3.61 × 10⁻² | UCHL1, MMP7, RPS6KA2, ABCA1 |
| Modification | | |
| RNA Damage and Repair | 2.13 × 10⁻²-2.13 × 10⁻² | ILIA |
| RNA Post-Transcriptional | 2.13 × 10⁻²-2.13 × 10⁻² | IL1A |
| Modification | | |

The corresponding control values can be the median amount of the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B gene products present in samples of similar origin as the test sample harvested from individuals without AMD. When the diagnostic method is for predicting whether an individual with the dry form of age-related macular degeneration is at risk of developing the wet form of age-related macular degeneration, the control value can be the median amount of the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B gene products present in samples of similar origin as the test sample harvested from individuals diagnosed as having the dry form of age-related macular degeneration.

The test sample can be any appropriate sample, for example, a tissue or body fluid sample. The body fluid sample, for example, can be selected from blood, serum, plasma, lacrimal fluid, vitreous, aqueous, and synovial fluid. The tissue sample, for example, can be selected from the group consisting of conjunctiva, cornea, sclera, uvea, retina, choroid, neovascular tissue, and optic nerve. The tissue sample can also include a plurality of cells, for example, 10-1000 cells, harvested from one of the foregoing tissues.

### A. Protein Detection of Gene Products

The presence and/or amount of a marker protein, for example, the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B protein, in a sample may be detected, for example, by combining the sample with a binding moiety capable of binding specifically to the marker protein. The binding moiety may comprise, for example, a member of a ligand-receptor pair, i.e., a pair of molecules capable of specific binding interactions. The binding moiety may comprise, for example, a member of a specific binding pair, such as antibody-antigen, enzyme-substrate, nucleic acid-nucleic acid, protein-nucleic acid, protein-protein or other specific binding pairs known in the art. Binding proteins may be designed which have enhanced affinity for the marker protein. Optionally, the binding moiety may be linked with a detectable label, such as an enzymatic, fluorescent, radioactive, phosphorescent or colored particle label. The labeled complex may be detected, e.g., visually or with the aid of a machine, for example, a spectrophotometer or other detector.

The marker proteins also may be detected using one- and two-dimensional gel electrophoresis techniques available in the art, such as those disclosed, for example, in Sambrook and Maniatis et al. eds. (1989) Molecular Cloning: A Laboratory Manual, Cold Spring Harbor Press. In one-dimensional gel electrophoresis, the proteins are usually separated according to their molecular weight. In two-dimensional gel electrophoresis, the proteins are first separated in a pH gradient gel according to their isoelectric point. The resulting gel then is placed on a second polyacrylamide gel, and the proteins separated according to molecular weight (see, for example, O'Farrell (1975) J. BIOL. CHEM. 250: 4007-4021).

The resulting gel pattern may then be compared with a standard gel pattern derived from a control sample (harvested, for example, from an individual without the angiogenic disorder, for example, without the ocular disorder, such as age-related macular degeneration, that is under study or from an individual with the dry form of age-related macular degeneration, as the case may be) and run under the same or similar conditions. The standard may be stored or obtained in an electronic database of electrophoresis patterns. The presence of a greater amount of a CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, and/or NALP1 protein or a decreased amount of a ROBO1, RORA, IGHM, NLRP2, PKP2, PLA2G4A, TANC1, UCHL1, ABCA1, VCAN, and/or FAM38B protein in the two-dimensional gel of the test sample compared to a control provides an indication that the individual has, or is at risk of developing, the disorder under study. The detection of two or more proteins in the two-dimensional gel electrophoresis pattern further enhances the accuracy of the assay. For example, assaying for an increased amount of one, two, three, four, five, six, or more of the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, and NALP1 proteins and/or a decreased amount of one, two, three, four, or more of the ROBO1, RORA, IGHM, NLRP2, PKP2, PLA2G4A, TANC1, UCHL1, ABCA1, VCAN, and FAM38B proteins provides a stronger indication that the individual has or is at risk of developing the disorder under study.

Furthermore, a CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B protein in a sample may be detected using any of a wide range of immunoassay techniques available in the art such as enzyme linked immunosorbent assays (ELISAs), Western blots, immunoprecipitations and immunofluorescence. For example, the skilled artisan may take advantage of the sandwich immunoassay format to detect if an individual has or is at risk of developing one or more angiogenic disorders, for example, an ocular angiogenic disorder, for example, a disorder associated with choroidal neovascularization, for example, age-related macular degeneration. Alternatively, the skilled artisan may use conventional immuno-histochemical procedures for detecting the presence of CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and FAM38B in a tissue sample, for example, using one or more labeled binding proteins, for example, a labeled antibody.

In a sandwich immunoassay, two antibodies capable of binding the marker protein are used, e.g., one immobilized onto a solid support, and one free in solution and labeled with detectable chemical compound. Examples of chemical labels that may be used for the second antibody include radioisotopes, fluorescent compounds, and enzymes or other molecules which generate colored or electrochemically active products when exposed to a reactant or enzyme substrate. When a sample containing the marker protein is placed in this system, the marker protein binds to both the immobilized antibody and the labeled antibody, to form a “sandwich” immune complex on the support's surface. The complexed marker protein is detected by washing away non-bound sample components and excess labeled antibody, and measuring the amount of labeled antibody complexed to protein on the support's surface.

Both the sandwich immunoassay and the tissue immunohistochemical procedure are highly specific and very sensitive, provided that labels with good limits of detection are used. A detailed review of immunological assay design, theory and protocols can be found in numerous texts in the art, including Butt, ed. (1984) Practical Immunology, Marcel Dekker, New York and Harlow et al., eds. (1988) Antibodies, A Laboratory Approach, Cold Spring Harbor Laboratory.

In general, immunoassay design considerations include preparation of antibodies (e.g., monoclonal or polyclonal antibodies) having sufficiently high binding specificity for the marker or target protein to form a complex that can be distinguished reliably from products of nonspecific interactions. As used herein, the term “antibody” is understood to mean an intact antibody (for example, polyclonal or monoclonal antibody); an antigen binding fragment thereof, for example, an Fab, Fab′ and (Fab′)₂ fragment; and a biosynthetic antibody binding site, for example, an sFv, as described in U.S. Pat. Nos. 5,091,513; and 5,132,405; and 4,704,692. A binding moiety, for example, an antibody, is understood to bind specifically to the target, for example, the CREB5, CXCL13, ENPP2, FAM169A (also known as KIAA0888), IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2 (also known as NALP2), PKP2, PLA2G4A, TANC1, UCHL1, ABCA1, VCAN, or FAM38B protein, for example, when the binding moiety has a binding affinity for the target greater than about 10⁵ M⁻¹, more preferably greater than about 10⁷ M⁻¹.

Antibodies against the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, or FAM38B proteins which are useful in assays for detecting if an individual has or is at risk of developing age-related macular degeneration may be generated using standard immunological procedures well known and described in the art. (See, e.g., Butt, N. R., ed. (1984) Practical Immunology, Marcel Dekker, New York). Briefly, an isolated CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, or FAM38B protein or fragment thereof is used to raise antibodies in a xenogeneic host, such as a mouse, goat or other suitable mammal.

The CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, or FAM38B protein or fragment thereof is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt, N. R., ed. (1984) Practical Immunology, Marcel Dekker, New York.

### B. Nucleic Acid Detection of Gene Products

The presence and/or amount of a CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B nucleic acid molecule (including, for example, polymorphic variants, promoter regions, introns, exons, and untranslated regions of the genes and/or gene products, and/or fragments thereof), for example, a mRNA, encoding a CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B protein may be determined using a labeled binding moiety capable of specifically binding the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B nucleic acid, respectively. The binding moiety may comprise, for example, a protein, a nucleic acid or a peptide nucleic acid. Additionally, a target nucleic acid, such as an mRNA encoding CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B protein, may be determined by conducting, for example, a Northern blot analysis using labeled oligonucleotides, e.g., nucleic acid fragments, complementary to and capable of hybridizing specifically with at least a portion of a target nucleic acid.

More specifically, gene probes comprising complementary RNA or DNA to the target nucleotide sequences or mRNA sequences encoding the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and FAM38B proteins may be produced using established recombinant techniques or oligonucleotide synthesis. The probes hybridize with complementary nucleic acid sequences presented in the test sample, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be useful in standard hybridization assays. Choices of probe length and sequence allow one to choose the degree of specificity desired. Hybridization is carried out at from 50° to 65° C. in a high salt buffer solution, formamide or other agents to set the degree of complementarity required. Furthermore, the state of the art is such that probes can be manufactured to recognize essentially any DNA or RNA sequence. For additional particulars, see, for example, Berger et al. (1987) “Guide to Molecular Techniques,” METHODS OF ENZYMOL 152.

A wide variety of different labels coupled to the probes may be employed in the protein and nucleic acid assays described herein. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescence. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′hydroxyl termini within either strand of the double-stranded fragment. Simultaneously, an exonuclease removes the nucleotide residue from the 5′phosphoryl side of the nick. The sequence of replacement nucleotides is determined by the sequence of the opposite strand of the duplex. Thus, if labeled nucleotides are supplied, DNA polymerase will fill in the nick with the labeled nucleotides. For smaller probes, known methods involving 3′ end labeling may be used. Furthermore, there are currently commercially available methods of labeling DNA with fluorescent molecules, catalysts, enzymes, or chemiluminescent materials. Biotin labeling kits are commercially available. This type of system permits the probe to be coupled to avidin which in turn is labeled with, for example, a fluorescent molecule, enzyme, antibody, etc. For further disclosure regarding probe construction and technology, see, for example, Sambrook et al. (1982) Molecular Cloning, A Laboratory Manual Cold Spring Harbor, N.Y.

The oligonucleotide selected for hybridizing to the target nucleic acid, whether synthesized chemically or by recombinant DNA methodologies, is isolated and purified using standard techniques and then preferably labeled (e.g., with ³⁵S or ³²P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridization and washing conditions. Specific hybridization and washing conditions include hybridization in, for example, 50% formamide, 5×SSPE, 2×Denhardt's solution, 0.1% SDS at 42° C., as described in Sambrook et al. (1989) supra, followed by washing in, for example, 2×SSPE, 0.1% SDS at 68° C., and/or 0.1×SSPE, 0.1% SDS at 68° C. Other useful procedures known in the art include solution hybridization, and dot and slot RNA hybridization. Optionally, the amount of the target nucleic acid present in a sample is then quantitated by measuring the radioactivity of hybridized fragments, using standard procedures known in the art.

In addition, it is anticipated that using a combination of appropriate oligonucleotide primers, i.e., more than one primer, the skilled artisan may determine the level of expression of a target gene by standard polymerase chain reaction (PCR) procedures, for example, by quantitative PCR. Conventional PCR based assays are discussed, for example, in Innes et al. (1990) PCR Protocols; A guide to methods and Applications, Academic Press and Innes et al. (1995) PCR Strategies, Academic Press, San Diego, Calif. Alternatively, the level of gene expression of the CREB5, CXCL13, ENPP2, FAM169A, IGKV1-5, IL1A, MMP7, RGS13, RPS6KA2, UGT2B17, CRIM1, CXCR4, C5orf26, IGHG3, IGLJ3, SHQ1, DNAJC6, C6orf105, NALP1, ROBO1, RORA, IGHM, NLRP2, PKP2, PLA2G4A, TANC1, UCHL1, ABCA1, VCAN, and/or FAM38B genes in the test sample and a control sample can be quantified by Northern blot analysis as known in the art.

In light of the foregoing description, the specific non-limiting examples presented below are for illustrative purposes and not intended to limit the scope of the invention in any way.

### EXAMPLES

### Example 1

### Identification of Genes and Pathways Associated with AMD

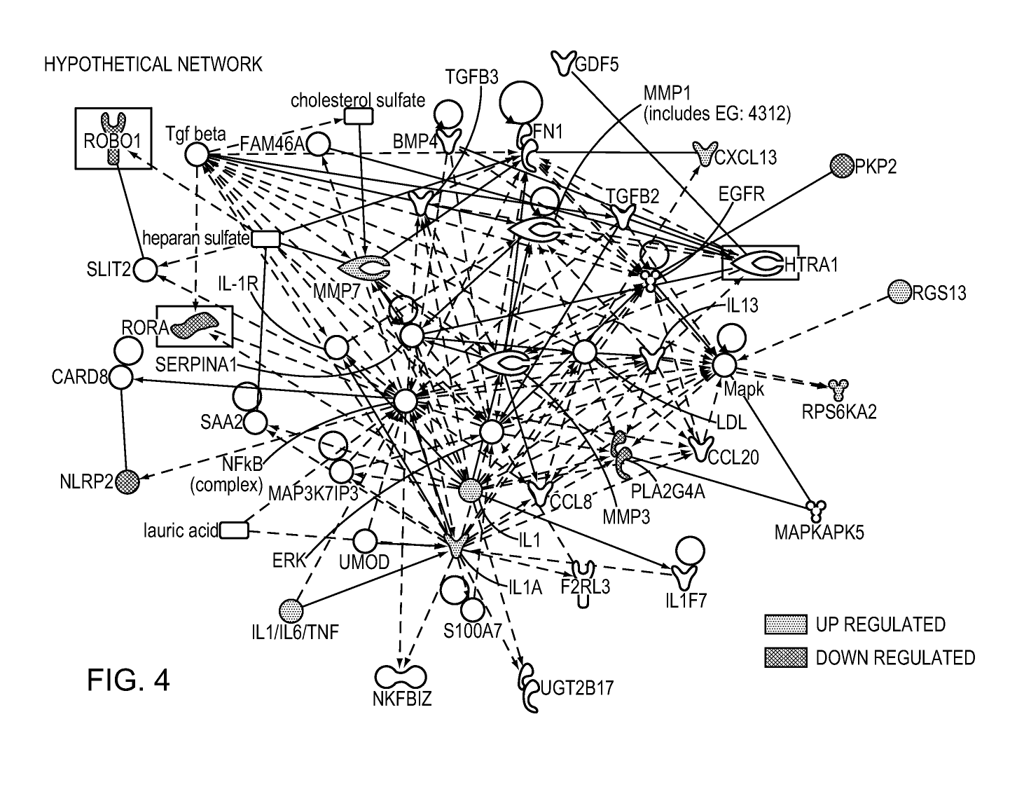

To identify novel genes and pathways associated with AMD, microarray gene expression was performed with Affymetrix U133A 2.0 PLUS on RNA from lymphoblastoid cell lines on patients with neovascular AMD and their unaffected siblings with no evidence of AMD (average age of subjects ≧75 years). This cohort has been previously described in detail (DeAngelis M M et al. (2007) OPHTHALMOLOGY; Zhang H et al., (2008) BMC MED GENET 9:51; DeAngelis M M et al. (2004) ARCH OPHTHALMOL 122:575-580; DeAngelis M M et al. (2007) ARCH OPHTHALMOL 125:49-54). Each sibling pair, of northern European ancestry, was matched for smoking history, age, gender, body mass index cardiovascular history, hypertension, and hypercholesterolemia, factors that could influence for factors that could influence their gene expression profiles. Genes (identified by at least 2 statistical methods after Bonferroni correction) that were statistically significant and had at least a 2-fold change between 9 sibpairs were chosen for further analysis. From our gene expression analysis coupled with our linkage analysis, along with pathways/network analysis (www.ingenuity.com/) a pathway/network of candidate genes was identified (FIGS. 3-4) (Silveira A C et al. (2010) VISION RESEARCH 50(7):698-715). These candidate genes include RAR-related orphan receptor A (“RORA”); cysteine-rich motor neuron 1, also known as cysteine rich transmembrane BMP regulator 1 (choroid like) (“CRIM1”); chemokine (C-X-C motif) receptor 4 (“CXCR4”); chromosome 5 open reading frame 26 (“C5orf26”); immunoglobulin heavy constant gamma 3 (G3m marker) (“IGHG3”); NACHT, leucine rich repeat and PYD containing 2, also known as NLR family, pyrin domain containing 2 or NLRP2 (“NALP2”); phospholipase A2, group IVA (cytosolic, calcium-dependent) (“PLA2G4A”); immunoglobulin lambda joining 3 (“IGLJ3”); regulator of G-protein signaling 13 (“RGS13”); chemokine (C-X-C motif) ligand 13 (B-cell chemoattractant) (“CXCL13”); ribosomal protein S6 kinase, 90 kDa, polypeptide 2 (“RPS6KA2”); matrix metalloproteinase 7 (matrilysin, uterine), also known as matrix metallopeptidase 7 (“MMP7”); Interleukin 1, alpha (“IL1A”); ATP-binding cassette, sub-family A, member 1 (“ABCA1”); Versican (“VCAN”); Small nucleolar RNAs of the box H/ACA family quantitative accumulation protein 1 (“SHQ1”); ubiquitin carboxyl-terminal esterase L1 (ubiquitin thiolesterase) (“UCHL1”); tetratricopeptide repeat, ankyrin repeat and coiled-coil containing 1 (“TANC1”); plakophilin 2 (“PKP2”); DnaJ (Hsp40) homolog, subfamily C, member 6 (“DNAJC6”); KIAA0888, also known as LOC26049 (“KIAA0888”); ectonucleotide pyrophosphatase/phosphodiesterase 2 (autotaxin) (“ENPP2”); family with sequence similarity 38, member B (“FAM38B”); chromosome 6 open reading frame 105 (“C6orf105”); and NLR family, pyrin domain containing 1 or NLRP1 (“NALP1”)

Within this network, the individual genes that were identified by gene expression are CXCL13, IL1A, MMP7, PKP2, PLA2G4A, NLRP2, RGS13, ROBO1, RORA, and RPS6KA2. This set of genes was simultaneously analyzed with linkage data previously obtained from our laboratory to investigate genomic convergence (Silveira A C et al. (2010) VISION RESEARCH 50(7):698-715).

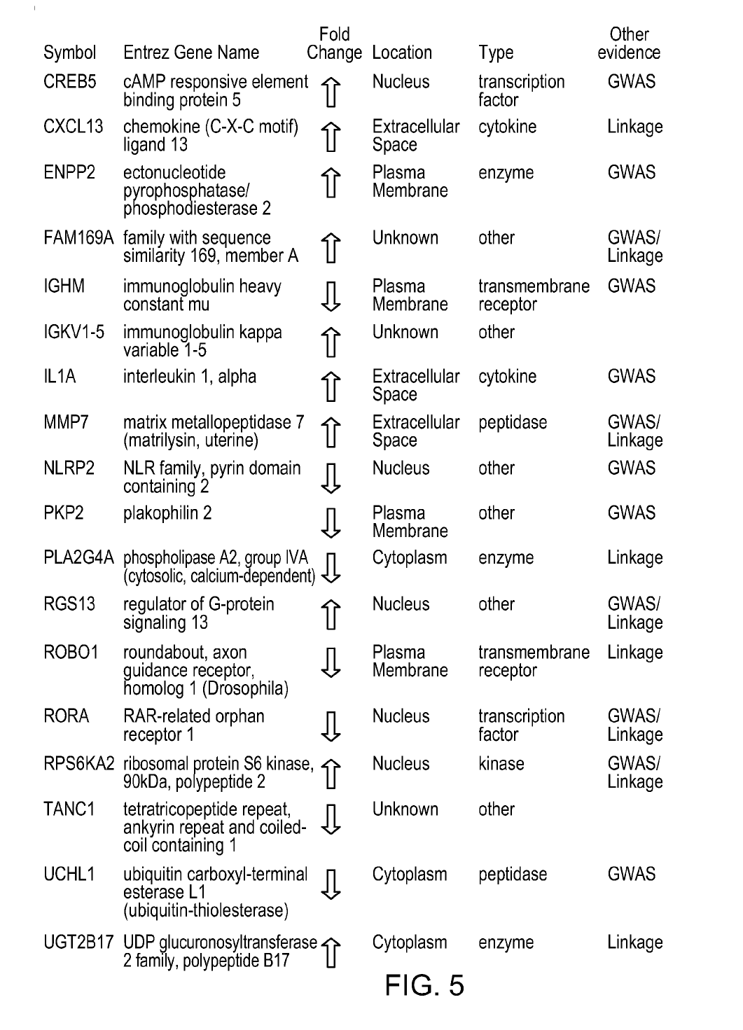

Based on the results of these studies, biological plausibility in AMD etiology, and significant decreased gene expression in affected patients compared to their unaffected siblings the candidate genes, RORA and ROBO1, were chose for further analysis. For example, in a family based cohort, ROBO1 was identified as containing a protective ROBO1 promoter haplotype that is significantly associated with neovascular AMD risk (p≦10⁻³) after correction for multiple testing. ROBO1, similar to RORA, was also observed to have decreased gene expression in patients when compared to their unaffected siblings (FIG. 5) and to interact with ARMS2/HTRA1. RT-PCR analyses were performed to confirm that both RORA and ROBO1 gene expression levels are down-regulated by 2 fold in affected patients compared to unaffected patients.

### Example 2

### Variants in the ROBO1 Gene Alter the Risk of AMD

This example describes the identification of alleles in ROBO1 that are associated with the development of AMD (e.g., dry and/or neovascular AMD). It also identifies the biological relevance of polymorphic variants in the ROBO1 gene, particularly, in the promoter of the ROBO1 gene.

Thirty-seven ROBO1 SNPs (Table3) were tested for their association with all AMD subtypes within the Sibling Cohort, using the minor allele, as defined as the allele occurring less frequently in the normal siblings. Tests for association were performed using the Likelihood Ratio Test (LRT) in the program UNPHASED, using the model for sibships. Of these 37 SNPs, 17 SNPs were identified as associated with All AMD subtypes when compared to their normal siblings, and also when looking at AMD as a quantitative trait (p<0.1). These same 37 SNPs were tested for their association with AMD subtypes in our unrelated cohort from Central Greece, and the results are shown here. One SNP that was significant in both cohorts, rs59931439, is found in intron 2 of the ROBO1 gene. In addition, numerous SNPS were significant in the Sibling Cohort when comparing the different AMD subtypes alone to normals.

TABLE 3

| SNP | Location^{a} | BP^{b} |
|---|---|---|
| rs723766 | 3′UTR | 78,657,774 |
| ROBO1_Ser162Ser | exon 3 | 78,987,766 |
| rs59931439 | intron 2 | 78,988,130 |
| rs1387665 | 5′ UTR/promoter | 79,429,811 |
| rs1546037 | 5′ UTR/promoter | 79,434,134 |
| rs4510348 | 5′ UTR/promoter | 79,438,446 |
| rs4680960 | 5′ UTR/promoter | 79,449,566 |
| rs13076006 | 5′ UTR/promoter | 79,452,636 |
| rs4680962 | 5′ UTR/promoter | 79,461,529 |
| rs13090440 | 5′ UTR/promoter | 79,465,496 |
| rs13058752 | 5′ UTR/promoter | 79,470,851 |
| rs7624099 | 5′ UTR/promoter | 79,475,253 |
| rs4513416 | 5′ UTR/promoter | 79,490,803 |
| rs4284943 | 5′ UTR/promoter | 79,495,754 |
| rs9810404 | 5′ UTR/promoter | 79,505,072 |
| rs9853257 | 5′ UTR/promoter | 79,524,548 |
| rs7640053 | 5′ UTR/promoter | 79,531,271 |
| rs7615149 | 5′ UTR/promoter | 79,537,773 |
| rs7622888 | 5′ UTR/promoter | 79,541,896 |
| rs4264688 | 5′ UTR/promoter | 79,546,348 |
| rs6548621 | 5′ UTR/promoter | 79,550,373 |
| rs7622444 | 5′ UTR/promoter | 79,557,927 |
| rs9832405 | 5′ UTR/promoter | 79,559,914 |
| rs7637338 | 5′ UTR/promoter | 79,560,604 |
| rs6548625 | 5′ UTR/promoter | 79,563,987 |
| rs7626242 | 5′ UTR/promoter | 79,567,274 |
| rs7623809 | 5′ UTR/promoter | 79,568,973 |
| rs9873952 | 5′ UTR/promoter | 79,573,229 |
| rs9871445 | 5′ UTR/promoter | 79,577,616 |
| rs4279056 | 5′ UTR/promoter | 79,581,250 |
| rs9848827 | 5′ UTR/promoter | 79,586,304 |
| rs9826366 | 5′ UTR/promoter | 79,588,523 |
| rs3923526 | 5′ UTR/promoter | 79,784,128 |
| rs1393370 | 5′ UTR/promoter | 79,790,293 |
| rs10865579 | 5′ UTR/promoter | 79,811,006 |
| rs9309833 | 5′ UTR/promoter | 79,811,719 |
| rs7629503 | 5′ UTR/promoter | 79,813,292 |

^{a}Location is based on the isoform b of the ROBO1 gene, whereas all the SNPs are located in intron 3 on the isoform a of the gene.
^{b}Base pair position (BP) was obtained using the NCBI B36 assembly of dbSNP b126.

ROBO1 SNPs that were individually identified as associated with a subject's risk of developing AMD are shown in Table 4. Values have been adjusted for age, sex and smoking.

TABLE 4

| | | Sibling Cohort | | Greek Cohort | |
|---|---|---|---|---|---|
| | Al- | AH AMD | Quantitative | All AMD | Quantitative |
| Name | lele | p value | p value | p value | p value |
| rs9826366 | C | 0.1521 | 0.0752 | 0.3411 | 0.9426 |
| rs6548625 | G | 0.2028 | 0.0959 | 0.5145 | 0.7893 |
| rs7622444 | C | 0.4297 | 0.0964 | 0.9874 | 0.7106 |
| rs7615149 | G | 0.1063 | 0.0305 | 0.5719 | 0.8199 |
| rs7640053 | G | 0.0851 | 0.0335 | 0.5113 | 0.9388 |
| rs9853257 | A | 0.1717 | 0.0511 | 0.5657 | 0.9972 |
| rs9810404 | G | 0.1089 | 0.0393 | 0.8742 | 0.8880 |
| rs4284943 | C | 0.1955 | 0.0877 | 0.9568 | 0.7037 |
| rs4513416 | A | 0.1425 | 0.0563 | 0.7666 | 0.9171 |
| rs7624099 | G | 0.1594 | 0.0444 | 0.6576 | 0.9621 |
| rs13058752 | C | 0.1519 | 0.0659 | 0.9496 | 0.7989 |
| rs13090440 | T | 0.0868 | 0.0239 | 0.8811 | 0.7965 |
| rs4680962 | A | 0.1294 | 0.0546 | 0.9493 | 0.7950 |
| rs13076006 | G | 0.1495 | 0.0598 | 0.6660 | 0.9758 |
| rs4680960 | A | 0.1598 | 0.0685 | 0.9275 | 0.8149 |
| rs4510348 | A | 0.1235 | 0.0275 | 0.7516 | 0.9555 |
| rs59931439 | T | 0.0161 | 0.0049 | 0.0086 | 0.0268 |

Additional SNPs that were determined to be associated with AMD in the Sibling Cohort using the Likelihood Ratio Test (LRT) in the program UNPHASED include rs4279056, rs9871445, rs7637338, rs6548621, rs1546037, rs1387665, and rs4335725. Additional SNPs that were determined to be associated with AMD in the Greek Cohort using the Likelihood Ratio Test (LRT) in the program UNPHASED include rs730754, rs9848827, rs9832405, rs723766, rs9873952, rs7626242 and rs9832405.

### Example 3

### ROBO1 Haplotype Replication: Neovascular AMD vs. Dry AMD

Eighteen SNPs were identified as located in the promoter region of ROBO1 that were associated with Neovascular AMD when compared to siblings with Dry AMD. In order to further narrow down the region of association, sliding window haplotype analysis was performed using the SNPs p<0.1.

Table 5 identifies the location in base pairs and the gene location of certain ROBO1 SNPs identified as associated with AMD. The common and variant alleles are also provided for two cohorts (e.g., alleles in the Sibling Cohort includes 226 discordant and 87 concordantly affected sib pairs from New England and the alleles in the Greek Cohort include 261 unrelated subjects from central Greece (139 affected and 121 unaffected). Variant alleles for both the Sibling Cohort and the Greek Cohort are presented using the forward strand of the Ensembl DNA database.

TABLE 5

| | | | Alleles in | Alletes in |
|---|---|---|---|---|
| | Location | Location in | Sibling | Greek |
| SNP | (bp) | gene | Cohort | Cohort |
| rs7629503 | 79,813,292 | 5′/promoter | C > A | C > A |
| rs9309833 | 79,811,719 | 5′/promoter | T > C | T > C |
| rs10865579 | 79,811,006 | 5′/promoter | T > C | T > C |
| rs1393370 | 79,790,293 | 5′/promoter | G > A | G > A |
| rs3923526 | 79,784,128 | 5′/promoter | T > A | T > A |
| rs6548621 | 79,550,373 | 5′/promoter | C > T | C > T |
| rs7615149 | 79,537,773 | 5′/promoter | T > G | T > G |
| rs59931439 | 78,988,130 | intron 2 | C > T | C > T |

A haplotype in the Sibling Cohort (n=657) was identified that decreases risk of developing neovascular AMD in those siblings with dry AMD (see H4 in Table 6). The protective haplotype is defined by the alleles present at rs6548621 and rs7615149.

TABLE 6

| | ROBO1 | ROBO1 | | Odds | | Overall |
|---|---|---|---|---|---|---|
| Haplotype | rs6548621 | rs7615149 | Freq | Ratio | p value | p value |
| H1 | C | T | 0.613 | 1.000 | 0.0481 | 0.0278 |
| H2 | T | T | 0.002 | 0.000 | 0.3038 | |
| H3 | C | G | 0.074 | 1.059 | 0.1926 | |
| H4 | T | G | 0.310 | 0.863 | 0.0145 | |

This same haplotype block, containing SNPs rs6548621 and rs7615149, was also found to be significant in the Greek Cohort (see H2 in Table 7).

TABLE 7

| | ROBO1 | ROBO1 | | Odds | | Overall |
|---|---|---|---|---|---|---|
| Haplotype | rs6548621 | rs7615149 | Freq | Ratio | p value | p value |
| H1 | C | T | 0.581 | 1.000 | 0.7780 | 0.0174 |
| H2 | C | G | 0.075 | 0.351 | 0.0045 | |
| H3 | T | G | 0.344 | 1.196 | 0.1982 | |

Although the significant haplotype was not the same alleles as in the Sibling Cohort, this significant haplotype is defined by two SNPs helps us narrow down the ROBO1 gene from 1,155,518 base pairs to a 12,600 base pair region in the promoter of the ROBO1 gene for direct sequencing.

### Example 4

### ROBO1 Statistical Interaction with RORA and HTRA1

Because ROBO1 was hypothesized to be in a network with RORA and ARMS2/HTRA, the genotyped SNPs in ROBO1 were tested for their statistical interaction with SNPS in the RORA gene and ARMS2/HTRA1 loci. Using a test for gene-gene interaction in the program UNPHASED, SNPs in the promoter of the ROBO1 gene were found that significantly interacted with RORA rs8034864 and HTRA1 promoter SNP rs2672598 in both the Sibling Cohort and the Greek Cohort.

Five SNPs (rs730754, rs8034864, rs12900948, rs17237514, rs4335725) in RORA that previously showed association with neovascular AMD in three diverse cohorts and 16 SNPs in ROBO1 that were moderately significant in the family cohort (P<0.05) were used to test gene-gene interaction. Tests of all models including one of the 16 ROBO1 SNPs, one of the 5 RORA SNPs and an interaction term in the two cohorts analyzed separately using the program UNPHASED revealed significant interaction between 9 SNPs in ROBO1 and rs8034864 in RORA after adjustment for multiple testing (meta P<6×10⁻⁴). No other SNPs in RORA showed significant interaction with ROBO1 SNPs at the permuted significance threshold of P<0.001. These findings suggest that the effects of the ROBO1 and RORA genes on neovascular AMD risk are not independent.

Table 8 shows the statistical interaction of ROBO1 SNP rs9309833 with RORA SNP rs8034864 (Sibling Cohort, p=0.0027; Greek Cohort, p=0347). Table 8 also shows the statistical interaction of ROBO1 SNPs rs7629503, rs10865579, rs1393370, rs3923526 with HTRA1 SNP rs2672598.

TABLE 8

| | RORA rs8034864 (C/A) | | HTRA1 rs2672598 (C/T) | |
|---|---|---|---|---|
| | SIBS | GREEKS | SIBS | GREEKS |
| ROBO1 SNP | “A” | “A” | “C” | “T” |
| rs7629503 “A” | 0.0507 | 0.4765 | 0.0201 | 0.0152 |
| rs9309833 “C” | 0.0027 | 0.0347 | 0.0269 | 0.0741 |
| rs10865579 “C” | 0.0401 | 0.3620 | 0.0163 | 0.0110 |
| rs1393370 “A” | 0.0040 | 0.1416 | 0.0077 | 0.0059 |
| rs3923526 “A” | 0.0040 | 0.1755 | 0.0078 | 0.0108 |

This statistical interaction provides some evidence of these genes interacting and operating within the same pathway to underlie AMD pathophysiology.

### Example 5

### Association of ROBO1 SNPs with Wet and/or Dry AMD

Association of ROBO1 SNPs with wet and/or dry AMD was further investigated by including data from a third cohort, the Nurses' Health Study and Health Professionals Follow-up Study (NHS-HPFS), in addition to The New England Sibling Cohort and the Greek Cohort. A description of the three cohorts (the Sibling Cohort, the Greek Cohort, and the NHS-HPFS cohort) is shown in Table 9. All analyses included age and sex distribution as covariates in order to control for their confounding effects. Details of recruitment, diagnostic criteria and subject classification for the NESC are described elsewhere (Silveira A C et al. (2010) VISION RESEARCH 50(7):698-715; DeAngelis et al. (2007) ARCH. OPHTHALMOL 125: 49-54). In brief, at least one individual from each family had the neovascular (wet) form of AMD in at least one eye after excluding patients with a retinal pigment epithelium detachment, myopia, ocular histoplasmosis syndrome, angioid streaks, choroidal rupture, any hereditary retinal diseases other than AMD, and previous laser treatment for retinal conditions other than AMD. A total of 352 wet AMD probands, 106 early/intermediate dry probands (Age Related Eye Disease Study [AREDS] category 2 and 3), and 198 normal siblings from 284 families comprising 352 wet AMD sibpairs and 76 early/intermediate dry sibpairs were available for this study. All but 87 of the sibpairs were discordant for AMD. The GREEK cohort was enrolled at the University Hospital of Larissa outpatient medical clinics in central Greece. The diagnosis of AMD in this cohort was confirmed by optical coherence tomography and Fluorescein angiography (Silveira A C et al. (2010) VISION RESEARCH 50(7):698-715; DeAngelis et al. (2007) ARCH. OPHTHALMOL 125: 49-54). A total of 139 wet AMD cases, 68 early and intermediate dry AMD cases, and 213 controls with normal macula were available after excluding patients with geographic atrophy. The NHS-HPFS comprised 1,070 controls, 164 wet AMD cases, and 293 dry AMD cases. Two different definitions were used for affection status, wet AMD and dry AMD, after excluding patients with geographic atrophy (Schaumberg et al. (2010) ARCH. OPHTHALMOL 128: 1462-1471).

TABLE 9
Description of Datasets

| | AMD | | |
|---|---|---|---|
| Study and Description | Controls | Wet AMD | Dry AMD |
| NESC | | | |
| Total, N | 198 | 352 | 106 |
| Average age at exam (SD) | 75.40 (8.25) | 73.80 (7.77) | 76.65 (12.32) |
| Gender (% of female) | 56.1% | 59.4% | 65.1% |
| Greek | | | |
| Total, N | 213 | 139 | 68 |
| Average age at exam | 73.78 (7.25) | 76.33 (7.49) | 74.44 (7.99) |
| (years) | | | |
| Gender (% of female) | 53.1% | 58.8% | 54.7% |
| NHS/HPFS | | | |
| Total, N | 1070 | 164 | 293 |
| Average age at exam | 60.21 (5.9) | 61.07 (6.0) | 59.53 (5.7) |
| (years) | | | |
| Gender (% of female) | 63.6% | 54.3% | 70.7% |

Abbreviations: SD, standard deviation; NESC, New England Sibling Cohort; Greek, central Greece cohort; NHS/HPFS, Nurses' Health Study (NHS) and Health Professionals Follow-up Study (HPFS).

Initially, genotyping was performed with tagging single nucleotide polymorphisms (SNPs) from the ROBO1 gene. To assess variation within this gene, tag SNPs were chosen to span the ROBO1 gene using data from the HapMap (www.hapmap.org/) after applying for the following criteria: 1) minor allele frequency was greater than 10%, 2) linkage disequilibrium (LD; r²) was at least 0.8, and 3) tagged for at least 6 other SNPs. These SNPs were genotyped using a combination of Sequenom and TaqMan. For the SNPs genotyped via Sequenom, multiplex PCR assays were designed using Sequenom SpectroDESIGNER software (version 3.0.0.3) (Sequenom, San Diego, Calif.) by inputting sequence containing the SNP site and 100 base pair (bp) of flanking sequence on either side of the SNP. Briefly, 10 ng of genomic DNA was amplified in a 5 uL reaction containing 1× HotStar Taq PCR buffer (Qiagen, Valencia, Calif.), 1.625 mM MgCl2, 500 uM each dNTP, 100 nM each PCR primer, 0.5 U HotStar Taq (Qiagen). The reaction was incubated at 94° C. for 15 minutes followed by 45 cycles of 94° C. for 20 seconds, 56° C. for 30 seconds, 72° C. for 1 minute, followed by 3 minutes at 72° C. Excess dNTPs were then removed from the reaction by incubation with 0.3 U shrimp alkaline phosphatase (USB, Cleveland, Ohio) at 37° C. for 40 minutes followed by 5 minutes at 85° C. to deactivate the enzyme. Single primer extension over the SNP was carried out in a final concentration of between 0.625 uM and 1.5 uM for each extension primer (depending on the mass of the probe), iPLEX termination mix (Sequenom) and 1.35 U iPLEX enzyme (Sequenom) and cycled using a two-step 200 short cycles program; 94° C. for 30 seconds followed by 40 cycles of 94° C. for 5 seconds, 5 cycles of 52° C. for 5 seconds, and 80° C. for 5 seconds, then 72° C. for 3 minutes. The reaction was then desalted by addition of 6 mg cation exchange resin followed by mixing and centrifugation to settle the contents of the tube. The extension product was then spotted onto a 384 well SpectroCHIP before being flown in the MALDI-TOF mass spectrometer. Data was collected, real time, using SpectroTYPER Analyzer 3.3.0.15, SpectraAQUIRE 3.3.1.1 and SpectroCALLER 3.3.0.14 (Sequenom). Additionally, to ensure data quality, genotypes for each subject was also checked manually. For the SNPs genotyped via TaqMan, either TaqMan Pre-Designed SNP Genotyping Assays or Custom TaqMan SNP Genotyping Assays (Applied Biosystems) kits were ordered (for listing of SNPs and probes, see Table 10). The 40× stock of the probes were diluted to 16× with 0.5× tris-EDTA and stored at −20° C. The amplification reaction was carried out in a total reaction volume of 16.25 μL containing 2.5 μL DNA (10 ng), 1.25 μL of 16× probe, and 12.5 μL of TaqMan Genotyping Master Mix. Sample DNA was amplified using the following reaction: 1 min at 60° C., 10 min at 95° C., and 40 cycles of 15 sec. at 92° C. and 1 min at 60° C. The amplification reaction is carried out on thermocyclers and then fluorescence is measured on the ABI 7500 Real-Time PCR System by which the genotypes are analyzed with the accompanying software, or, in some cases, manually.

TABLE 10

| SNP | Probe Name |
|---|---|
| rs9832405 | C 11523693_10 |
| rs7622444 | C 29805155_20 |
| rs6548621 | C 11523723_10 |
| rs7615149 | C 409099_10 |
| rs4513416 | C 307534_10 |
| rs59931439 | C 25632225_10 |
| rs1387665 | AHX0JQB |

All genotyped SNPs met quality control thresholds of call rate of at least 90% and being in Hardy-Weinberg equilibrium (HWE) (P>0.01). LD among ROBO1 SNPs was evaluated using the HapMap CEU reference population. At least one SNP from each haplotype block, delineated on the basis of pairwise estimates of LD (r²)>0.5, was further analyzed under different genetic models and in the interaction analyses. This SNP selection scheme both sufficiently accounts for the potential contribution of ROBO1 individually and through interaction with RORA to AMD risk, and minimizes the penalty of multiple testing.

Based on the location of the significant SNPs found in the initial screen of ROBO1, direct sequencing was also performed on the promoter and exons 1, 2, and 3 in order to discover novel variation. For these reactions, oligonucleotide primers were selected using the Primer3 program (found at the website “primer3.sourceforge.net/”) to encompass the SNP and flanking intronic sequences. All PCR assays were performed using genomic DNA fragments from 20 ng of leukocyte DNA in a solution of 10 PCR buffer containing 25 mM of MgCl2, 0.2 mM each of dATP, dTTP, dGTP, and dCTP, and 0.5 U of Taq DNA polymerase (USB Corporation). Five molar betaine was added to the reaction mix for rs2414687 (Sigma-Aldrich, St. Louis, Mo.). The temperatures used during the polymerase chain reaction were as follows: 95° C. for 5 min followed by 35 cycles of 58° C. for 30 s, 72° C. for 30 s and 95° C. for 30 s, with a final annealing at 58° C. for 1.5 min and extension of 72° C. for 5 min. For sequencing reactions, PCR products were digested according to manufacturer's protocol with ExoSAP-IT (USB Corporation) then were subjected to a cycle sequencing reaction using the Big Dye Terminator v 3.1 Cycle Sequencing kit (Applied Biosystems, Foster City, Calif.) according to manufacturer's protocol. Products were purified with Performa DTR Ultra 96-well plates (Edge Biosystems, Gaithersburg, Md.) in order to remove excess dye terminators. Samples were sequenced on an ABI Prism 3100 DNA sequencer (Applied Biosystems). Electropherograms generated from the ABI Prism 3100 were analyzed using the Lasergene DNA and protein analysis software (DNASTAR, Inc., Madison, Wis.). Electropherograms were read independently by two evaluators without knowledge of the subject's disease status. All patients were sequenced in the forward direction (5′-3′), unless variants or polymorphisms were identified, in which case confirmation was obtained in some cases by sequencing in the reverse direction. Sequence notation throughout this example corresponds to the NCBI B36 assembly of dbSNP b126.

Linkage disequilibrium (LD) among the genotyped SNPs was determined using Haploview (version 4.2; www.broadinstitute.org/scientific-community/science/programs/medical-and-population-genetics/haploview/haploview). ROBO1 SNPs were tested for association with wet and dry AMD classification groups in the discovery cohorts using a logistic regression approach under an additive model including age and sex as covariates. Generalized Estimating Equations (GEE) were used in the analysis of the family dataset to account for familial correlations (Chen et al. (2010) BIOINFORMATICS 26: 580-581) and a generalized linear model approach was used for the unrelated cohorts. All analyses were performed using the R package (R2.2.1; www.r-project.org/). Haplotype analysis was performed using UNPHASED (version 3.1.4; found at website “homepages.lshtm.ac.uk/frankdudbridge/software/unphased/”) (Dudbridge (2003) GENET. EPIDEMIOL 25: 115-121; Dudbridge (2008) HUM. HERED 66: 87-98) which can account for family-based data. Association results obtained from individual datasets were combined by meta-analysis using the inverse variance method implemented in the software package METAL (www.sph.umich.edu/csg/abecasis/Metal/) (Willer et al. (2010) BIOINFORMATICS 26: 2190-2191). Effect sizes were determined by summing the regression coefficients weighted by the inverse variance of the coefficients. Significant findings from the combined discovery cohorts were evaluated for association in the replication sample. Results from the three cohorts were combined by meta-analysis. SNPs with nominally significant P values (<0.05) in the combined sample (meta P) were further tested under dominant and recessive models.

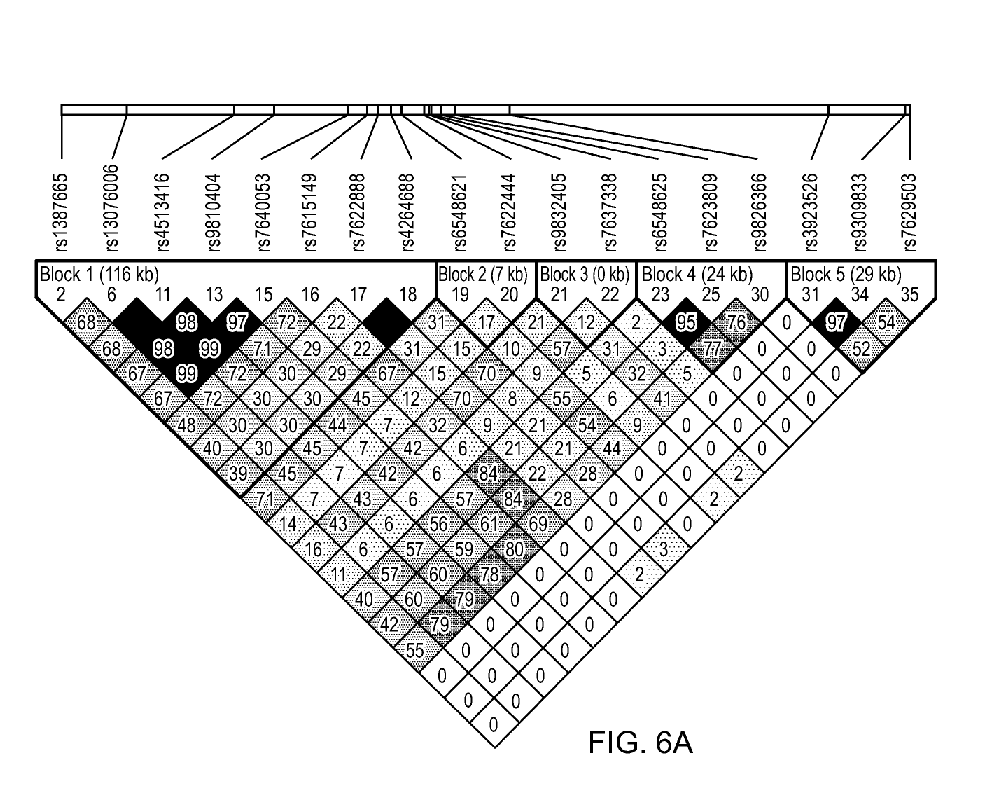

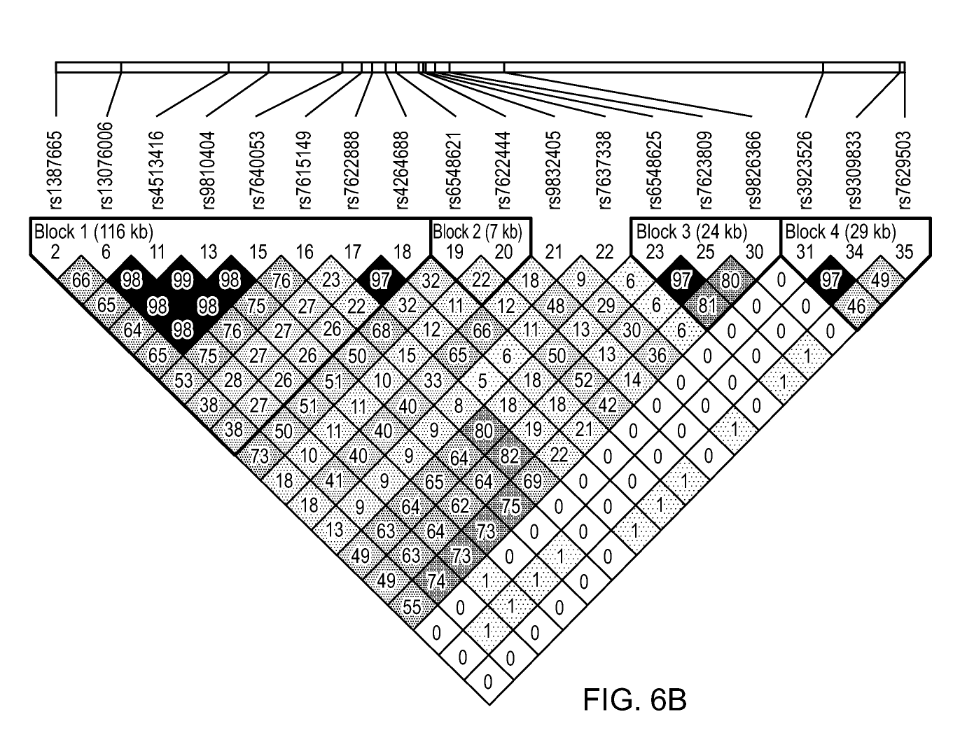

The analysis separated two subtypes of AMD (wet and dry) from all AMD or advanced AMD, to investigate multiple variants that may be involved in the early/intermediate or advanced/severe neovascular AMD subtype. Analysis of linkage disequilibrium (LD) among ROBO1 SNPs revealed a minimum of three distinct haplotype blocks (FIG. 6): the first block encompassing the region between rs1387665 and rs4264688, the second between rs6548621 to rs9826366, and the third block (identified as block 5 in FIG. 6A and block 4 in FIG. 6B) including rs3923526, rs9309833, and rs7629503.

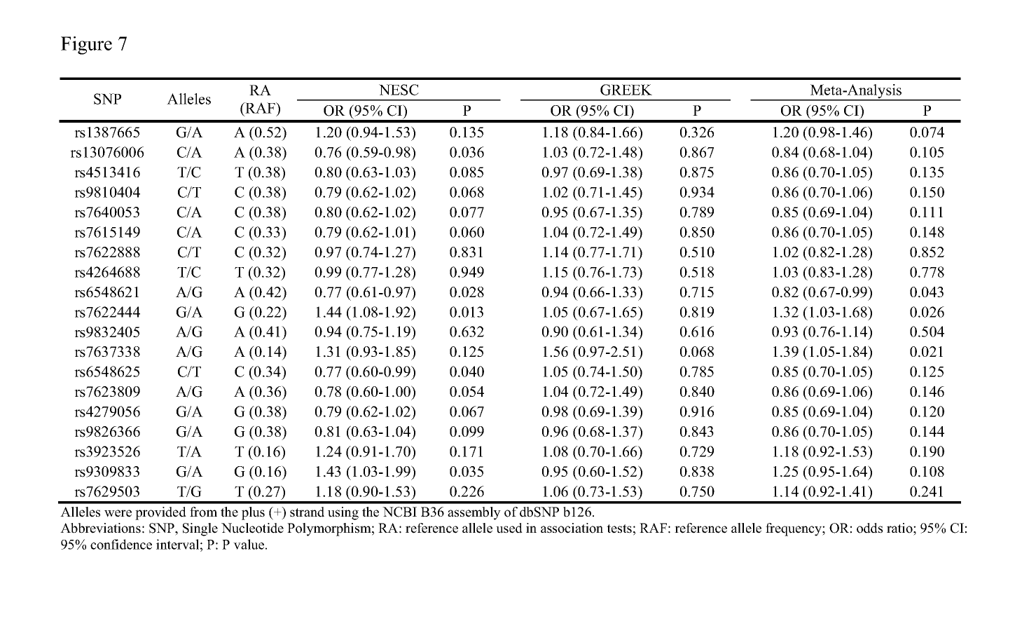

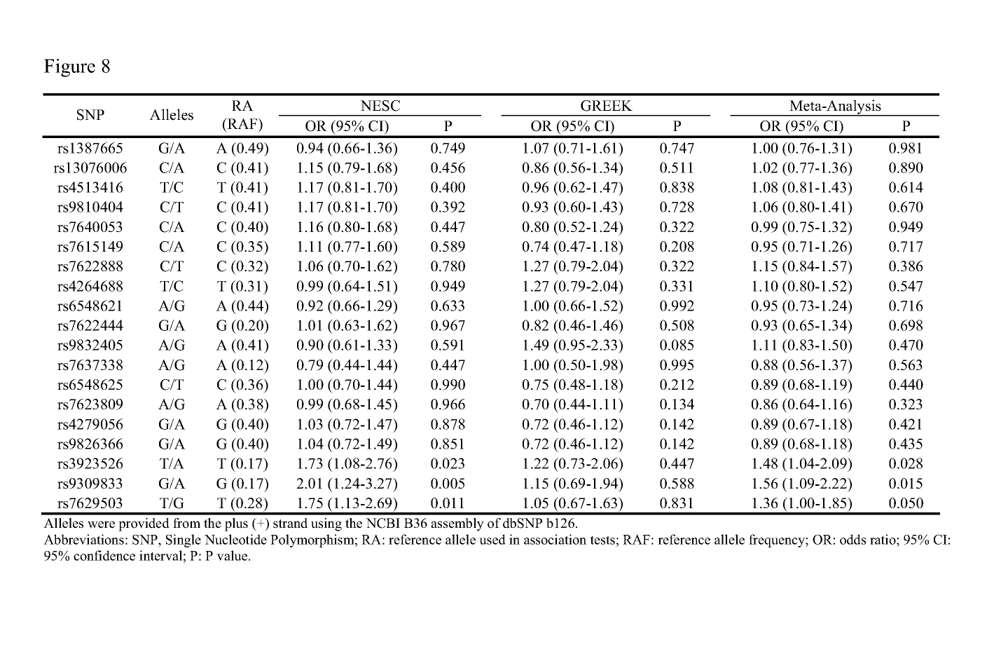

Of the 37 SNPs discussed in Example 2, 19 tag SNPs residing upstream of the isoform b and in intron 3 of the isoform a in the human sequence were chosen for further study (FIG. 7). Association with the neovascular (wet) form of AMD and dry AMD (Age Related Eye Disease Study [AREDS] category 2 and 3) was determined. In the Sibling Cohort, five of the 19 ROBO1 SNPs (rs13076006, rs6548621, rs7622444, rs6548625, rs9309833) were associated with wet AMD at a nominal significance level at P<0.05 (FIG. 7). None of these SNPs were significantly associated with wet AMD in the Greek Cohort (P>0.05). Meta-analysis of the two cohorts revealed three SNPs (rs6548621, rs7622444, and rs7637338) from the middle LD block showed mild association (most significant SNP: rs7637338 with P=0.021). The minor allele A of rs7637338 showed increased risk with an odds ratio (OR) of 1.39 (95% confidence interval [CI]=1.05-1.84). An odds ratio (OR) above 1 generally indicates that a variant is associated with risk and an OR below 1 generally indicates that a variant is protective. Three 5′ SNPs (rs3923526, rs9309833, and rs7629503) were moderately significant with dry AMD in the Sibling Cohort, of which rs9309833 was the most significant (P=0.005) (FIG. 8). Although these SNPs were not significant at P<0.05 in the Greek Cohort, the direction of effect was the same for each (FIG. 8) and the SNP rs9309833 remained significant in meta-analysis (meta P=0.015). The two most significant SNPs for wet AMD (rs7637338) and for dry AMD (rs9309833) are uncorrelated (FIG. 6) in both cohorts (r²<0.06), suggesting that these two signals are tagging independent causal variants in this gene.

These findings were extended to testing different genetic models with four SNPs covering each LD block and attempting to confirm the results in the NHS-NPFS replication cohort. Table 11 shows association results of ROBO1 SNPs for wet AMD or dry AMD in meta-analysis under the three different genetic models (additive, dominant, and recessive) from the combined dataset including the Sibling Cohort, the Greek Cohort, and the NHS-HPFS cohort. Association signals in the first block of ROBO1 for wet AMD were confirmed, with rs1387665 being the most significant under an additive model in meta-analysis of the three datasets (meta P=0.028; OR=1.18, CI=1.02-1.37). However, this SNP was not associated with dry AMD (meta P>0.14). In contrast, rs9309833 from the third block was more strongly associated with dry AMD (meta P=6×10⁻⁴; OR=2.54, CI=1.49-4.34) than with wet AMD (meta P=0.047; OR=1.88, CI=0.99-3.56) under a recessive model. The association signal with rs9309833 for dry AMD remained significant even after adjusting for testing multiple SNPs, models, and traits (threshold P=0.002 obtained with dividing 0.05 by 24 tests). There was no LD (r²=0) between rs1387665 and rs9309833 in all cohorts. These results suggest that there may be two or more independent causal variants residing in the different regions of the ROBO1, and the genetic models governing the effect of these variants may differ for wet and dry AMD.

TABLE 11

| | |
|---|---|
| Wet AMD | Dry AMD |

| SNP | Model | RA | OR (95% CI) | P | OR (95% CI) | P |
|---|---|---|---|---|---|---|
| rs1387665 | Add | A | 1.18 (1.02-1.37) | 0.0281 | 1.10 (0.95-1.28) | 0.2179 |
| | Dom | | 1.23 (0.96-1.58) | 0.1027 | 1.21 (0.94-1.55) | 0.1462 |
| | Rec | | 1.28 (1.00-1.64) | 0.0490 | 1.08 (0.84-1.38) | 0.5413 |
| rs4513416 | Add | T | 0.88 (0.75-1.02) | 0.0979 | 0.93 (0.80-1.09) | 0.3680 |
| | Dom | | 0.81 (0.64-1.02) | 0.0687 | 0.91 (0.73-1.14) | 0.4212 |
| | Rec | | 0.90 (0.67-1.19) | 0.4486 | 0.91 (0.68-1.22) | 0.5151 |
| rs7622444 | Add | G | 1.11 (0.91-1.36) | 0.2870 | 0.90 (0.73-1.11) | 0.3093 |
| | Dom | | 1.05 (0.83-1.32) | 0.6948 | 0.82 (0.64-1.04) | 0.0969 |
| | Rec | | 1.74 (0.95-3.19) | 0.0703 | 1.66 (0.91-3.02) | 0.0993 |
| rs9309833 | Add | G | 1.18 (0.96-1.44) | 0.1150 | 1.33 (1.09-1.61) | 0.0041 |
| | Dom | | 1.13 (0.90-1.43) | 0.3000 | 1.26 (1.01-1.59) | 0.0451 |
| | Rec | | 2.00 (1.01-3.96) | 0.0465 | 2.54 (1.49-4.34) | 6 × 10⁻⁴ |

Alleles were provided from the plus (+) strand using the NCBI B36 assembly of dbSNP b126.
Abbreviations:
SNP, Single Nucleotide Polymorphism;
RA: reference allele used in association tests;
OR: odds ratio;
95% CI: 95% confidence interval;
P: P value.

### Example 6

### ROBO1 Statistical Interaction with RORA and HTRA1 in Wet and/or Dry AMD

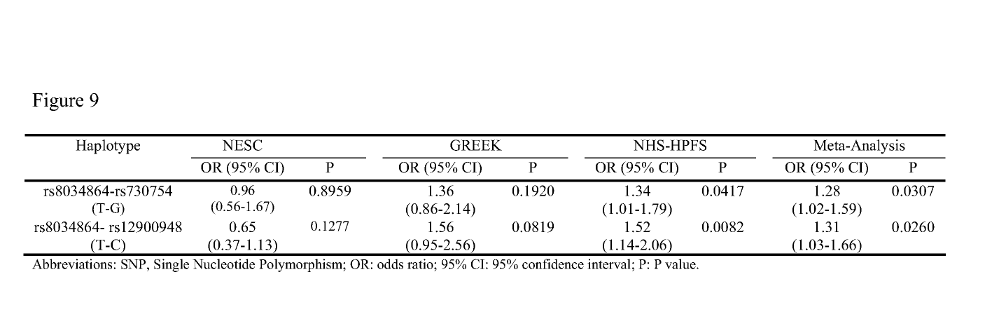

Further analysis of the interaction between ROBO1 an RORA was performed which included data from the NHS-NPFS cohort. In addition, the study separated two subtypes of AMD (wet and dry) from all AMD or advanced AMD, to investigate multiple variants that may be involved in the early/intermediate or advanced/severe neovascular AMD subtype. To perform the interaction analysis, four ROBO1 tagging SNPs (rs1387665, rs4513416, rs7622444, and rs9309833) in a region that likely harbors alternative splice sites were selected based on LD patterns in the region (FIG. 6). Association of RORA SNPs for wet AMD was confirmed using haplotype analysis using the UNPHASED program. Among the previously reported significant RORA SNPs for wet AMD (rs4335725 and rs8034864), haplotypes containing rs8034864 had the most consistent evidence of association in meta-analysis (FIG. 9). Therefore, additive models were constructed, including one of four significant ROBO1 SNPs, the RORA SNP (rs8034864), and an interaction term for the ROBO1 and RORA SNPs. In other words, interaction of each of four ROBO1 SNPs with a RORA SNP was assessed by comparing a baseline additive model, which includes an independent term for each SNP, to the full additive model which includes the SNP main effects plus an interaction term. Significant findings in the discovery datasets were tested for confirmation in the NHS-HPFS cohort. Using the estimates from the meta-analysis, probabilities from a full logistic model, Pₕ(X)=1/{1+exp[−(α+β₁SNP₁+[β₂SNP₂+β₃SNP₁×SNP₂)]}1/[1+e−(α+β₂SNP1+β₂SNP2+β₂SNP1×SNP2j], under the assumption of the same age and sex was calculated for each genotypic categories for wet and dry AMD and plotted against grouped genotypes from the two interacting SNPs. Other genetic models were not tested because of small sample sizes for many of the SNP×SNP genotype cells.

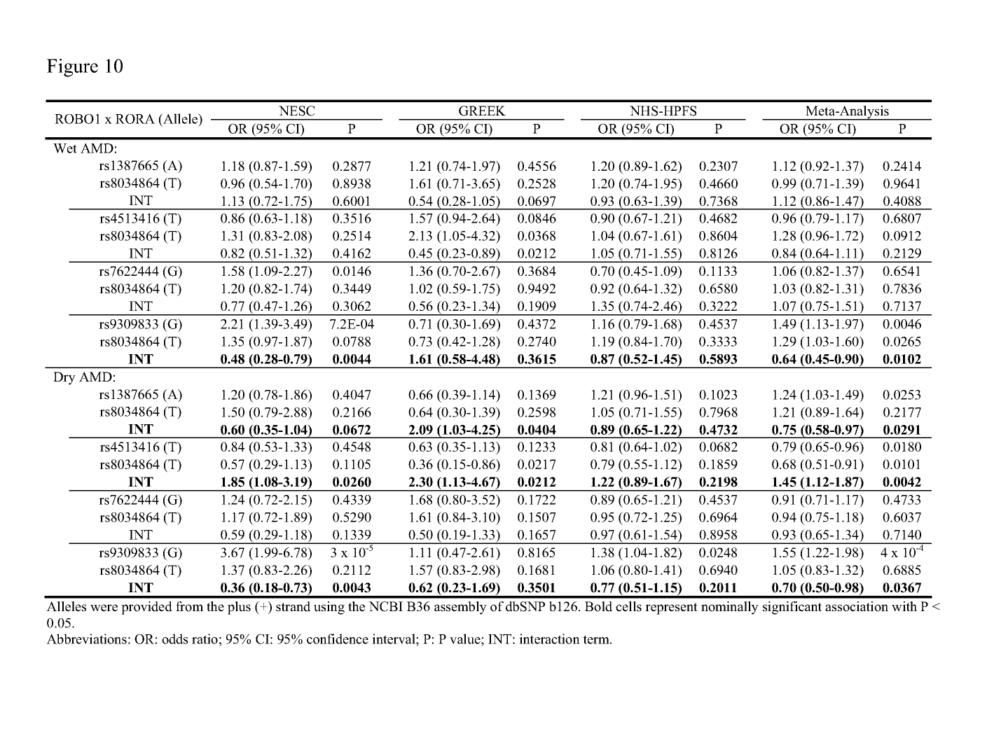

As shown in FIG. 10, interaction analysis was performed between RORA rs8034864 and each of four ROBO1 tagging SNPs (rs1387665, rs4513416, rs7622444, and rs9309833) for each cohort, for both wet and dry AMD. In addition, the data for all three cohorts was combined using meta-analysis for each combination of SNPs. Odds ratios (OR) and P values for each individual SNP as well as for the interaction are shown. An odds ratio (OR) above 1 generally indicates that a variant is associated with risk and an OR below 1 generally indicates that a variant is protective. A p-value <0.05 indicates a significant association. Rows showing significant associations are displayed in bold in FIG. 10. rs9309833 was shown to interact with RORA rs8034864 in both wet and dry AMD, and rs1387665 and rs4513416 were shown to interact with RORA rs8034864 in dry AMD, as discussed in more detail below.

Moderately significant interactions were found between RORA rs8034864 and ROBO1 SNPs for both wet and dry AMD (FIG. 10). The interaction of rs8034864 and rs4513416 from the ROBO1 gene remained significant (meta P for interaction=0.0042) after correction for testing eight interaction models (threshold P=0.006). There was also significant evidence of interaction between ROBO1 SNP rs9309833 and RORA SNP rs8034864 in affecting the risk of both wet (meta P for interaction=0.010) and early/intermediate dry AMD (meta P for interaction=0.037). The effect direction (i.e., whether associated with risk or with protection) of these significant SNPs and the pattern of their interactions for early/intermediate dry AMD were consistent in all datasets (FIG. 10).

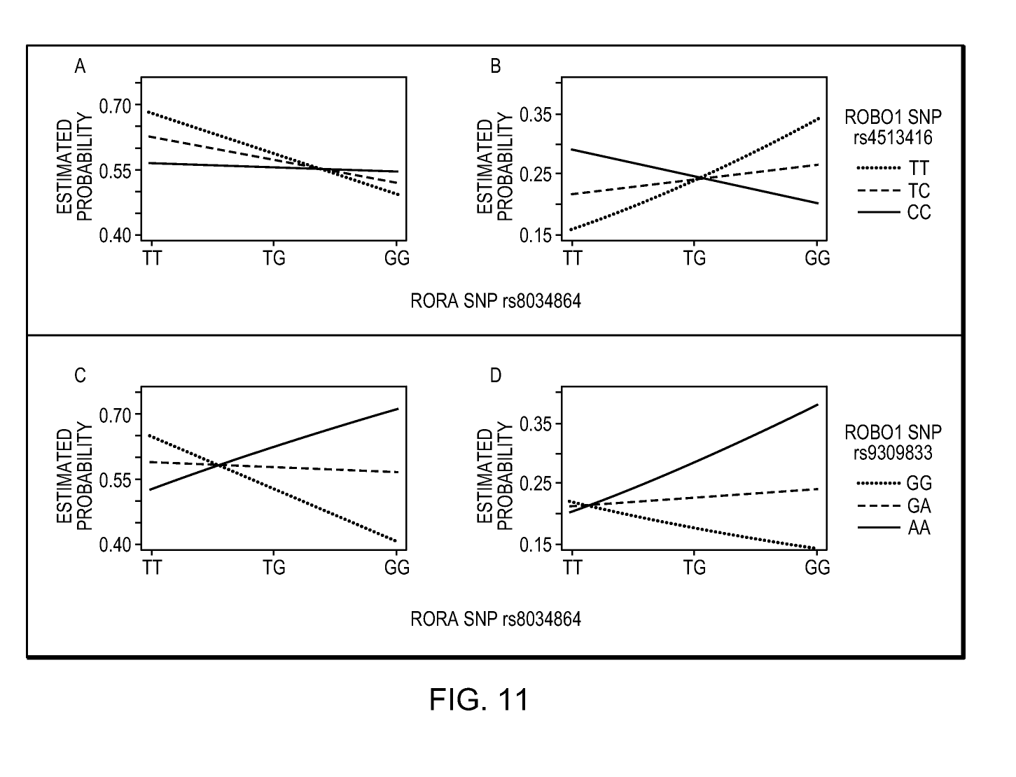

Analysis of the full logistic models (FIG. 11) revealed that comparing with the dosage effect of the rs4513416 C allele for wet AMD (FIG. 11A) that for early/intermediate dry AMD was modulated by the dose of the rs8034864 T allele (FIG. 11B). Interaction between ROBO1 SNP rs9309833 and RORA SNP rs8034864 was significant for both wet (FIG. 11C) and early/intermediate dry AMD (FIG. 11D) such that risk of AMD increased according to dose of the rs8034864 G allele among rs9309833 AA homozygotes, whereas AMD risk decreased according to dose of the rs8034864 G allele among rs9309833 GG homozygotes.

The study design is unique from others in that two subtypes of AMD were separated from all AMD or advanced AMD, to investigate multiple variants that may be involved in the early/intermediate or advanced/severe neovascular AMD subtype. This approach is supported by an illustration of a review (Hamdi et al. (2003) FRONT. BIOSCI 8: e305-314) that three different components of AMD, drusen formation, neovascularization, and RPE atrophy, have seen in many different complex diseases, implying that there may be independent underlying mechanisms to develop each of these components. A previous study also demonstrated that drusen formation may have both unique and shared underlying genetic mechanisms with intermediate or advanced AMD development (Jun et al. (2005) INVEST. OPHTHALMOL. VIS. SCI 46: 3081-3088). Specifically, this previous study showed that drusen formation as an intermediate stage of advanced AMD types identified previously known linkage signals for advanced AMD as well as novel peaks. One of the unique peaks for large drusen size is on chromosome 19q13.21, that is accounted for by the genotype of the APOE gene. This previous study further supports the results presented herein relating to differential association signals for wet and early/intermediate dry AMD. This hypothesis-driven, genomic convergent approach based on prior biological plausibility provided collective evidence from statistical tests and molecular experiments demonstrating another pathway underlying AMD pathogenesis.

### Example 7

### Gene Expression Profiling in Human Donor Eyes

To compare levels of expression of ROBO1 and RORA in AMD patients and controls, whole transcriptome expression profiles were obtained from 126 RPE-choroid and 118 retina punches (each 6 mm in diameter) obtained from the macular and extramacular regions of eyes from 66 human donors. These eyes were selected from a well-characterized repository including 3,903 donors collected over a 20 year period at the University of Iowa and St. Louis University by Dr. Hageman. Medical and ophthalmic histories, a family questionnaire, blood, and sera, were obtained from the majority of donors. Gross pathologic features, as well as the corresponding fundus photographs and angiograms, when available, of all eyes in this repository were read and classified by retinal specialists. Fundi and/or posterior poles were graded using a slightly modified version of two standardized classification systems, as published previously (Mullins et al. (2000) FASEB J 14: 835-846; Hageman et al. (2001) PROG RETIN EYE RES 20: 705-732; Chong et al. (2005) AM. J. PATHOL 166: 241-251; Anderson et al. (2002) AM. J. OPHTHALMOL 134: 411-431; Hageman et al. (2005) PROC. NATL. ACAD. SCI. U.S.A 102: 7227-7232). The ages of the donors ranged from 9 to 101 years; approximately 50% had documented clinical histories of AMD. RNA expression profiles were assessed using two-color, 44K Agilent Whole Genome in situ oligonucleotide microarray analysis and a universal reference RNA experimental design. The universal reference RNA consisted of a 1:1 pool of RPE-choroid and retina RNA generated from donors with and without AMD. After correcting for dye effects using LOWESS normalization, the net intensity values were determined and expressed as a percentage of the total array intensity. The ratios of the experimental and reference RNA signals were calculated, and then the normalized percent total of each experimental value was calculated by multiplication using the geometric mean of all determinations of each probe's reference RNA value. For those probes with replicates in the array, the average values were determined. Inter-array differences were further corrected by quantile normalization and probes that did not have net intensities values greater than six times the standard deviation of the background in at least 5% of the samples were omitted. This resulted in a final data set comprised of 28,127 unique probes. Expression of the ROBO1 and RORA genes was examined.

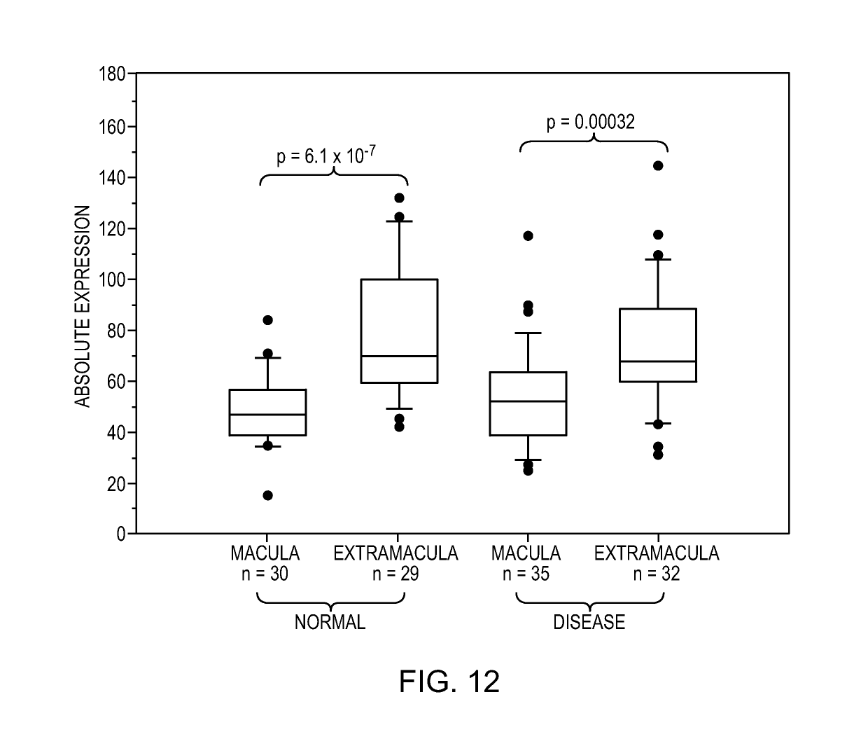

Expression of both ROBO1 and RORA was detected in the RPE-choroid and the retina. Of the genes examined in a whole transcriptome analysis of ocular tissues derived from 66 human donors, no significant association as a function of age was observed. Statistically significant differences in RORA expression were not observed (data not shown), but ROBO1 expression was significantly different between the macula and extramacula in both normal and AMD RPE-choroid (FIG. 12). This complements a previous finding in immortalized cell lines, which showed ROBO1 had decreased expression by at least two fold in index patients with neovascular AMD compared to their unaffected siblings (Silveira et al., (2010) VISION RESEARCH 50(7):698-715).

### INCORPORATION BY REFERENCE

The entire content of each patent and non-patent document disclosed herein is expressly incorporated herein by reference for all purposes, including Silveira et al., (2010) VISION RESEARCH 50(7):698-715.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method for determining a subject's risk of developing age-related macular degeneration, the method comprising detecting in a sample obtained from the subject the presence or absence of an allelic variant at a polymorphic site in the ROBO1 gene that is associated with risk of developing age-related macular degeneration.

2. The method of claim 1, comprising detecting the presence or absence of a risk variant at a polymorphic site in the ROBO1 gene, wherein, if the subject has the risk variant, the subject is more likely to develop age-related macular degeneration than a person without the risk variant.

3. The method of claim 2 wherein the polymorphic site comprises a site selected from the group consisting of rs9309833, rs4513416, rs1387665, rs7629503, rs3923526, rs7622444, and rs7637338.

4. The method of claim 1, wherein the polymorphic site is rs9309833.

5-6. (canceled)

7. The method of claim 1, wherein the polymorphic site is rs4513416.

8-9. (canceled)

10. The method of claim 1, wherein the polymorphic site is rs1387665.

11-12. (canceled)

13. The method of claim 1, wherein the polymorphic site is rs7629503.

14-15. (canceled)

16. The method of claim 1, wherein the polymorphic site is rs3923526.

17-18. (canceled)

19. The method of claim 1, wherein the polymorphic site is rs7622444.

20-21. (canceled)

22. The method of claim 1, wherein the polymorphic site is rs7637338.

23-24. (canceled)

25. The method of claim 1, comprising detecting the presence or absence of a protective variant at a polymorphic site in the ROBO1 gene, wherein, if the subject has the protective variant, the subject is less likely to develop age-related macular degeneration than a person without the protective variant.

26. The method of claim 25, wherein the polymorphic site comprises a site selected from the group consisting of rs7615149, rs6548621, rs59931439, rs13076006, and rs6548625.

27. The method of claim 1, wherein the polymorphic site is rs7615149.

28-29. (canceled)

30. The method of claim 1, wherein the polymorphic site is rs6548621.

31-32. (canceled)

33. The method of claim 1, wherein the polymorphic site is rs59931439.

34-35. (canceled)

36. The method of claim 1, wherein the polymorphic site is rs13076006.

37-38. (canceled)

39. The method of claim 1, wherein the polymorphic site is rs6548625.

40-41. (canceled)

42. The method of claim 1, comprising detecting the presence or absence of a variant at a polymorphic site in the ROBO1 gene, wherein, if the subject has the variant, the subject has an altered risk of developing age-related macular degeneration than a person without the variant.

43. The method of claim 25, wherein the polymorphic site comprises a site selected from the group consisting of ROBO1 Ser162Ser, rs10865579, rs1393370, rs7640053, rs13090440, rs4680962, rs4510348, rs9810404, rs7624099, rs9853257, rs4284943, rs13058752, rs4680960, rs1546037, rs4279056, rs9871445, rs9826366, rs9848827, rs9832405, rs723766, rs9873952, rs7626242, rs7622888, rs4264688, and rs7623809.

44. The method of claim 1, wherein the allelic variant defines a haplotype.

45. The method of claim 1, further comprising detecting the presence or absence of an allelic variant at a polymorphic site in a RORA gene.

46. The method of claim 45, wherein the polymorphic site in the RORA gene is rs8034864.

47. The method of claim 46, wherein the allelic variant defines a haplotype in the RORA gene.

48. The method of claim 47, wherein the haplotype in the RORA gene is defined by rs12900948, rs730754, and rs8034864.

49. The method of claim 48, further comprising detecting an adenine base or guanine base at rs12900948, an adenine or guanine base at rs730754, and a cytosine or adenine base at rs803486451.

50. The method of claim 47, wherein the haplotype in the RORA gene is defined by rs17237514 and rs4335725.

51. The method of claim 50, further comprising detecting an adenine base or guanine base at rs17237514 and an adenine or guanine base at rs4335725.

52-82. (canceled)


SEQUENCE LISTING (42 sequences)

SEQ ID NO: 1 - DNA, 6895 bp, Homo sapiens
cccgacttcactctctccctatttccccactcttaggtttaaaagtctgtcacctttcgcttggtttaaactcggaaaggtctcagtgcacagcaaagttgcagggctgcgtctgcactacggagcctctagattgctgaaaacagtcttatggaaggataacacattgtctgtcactggctggttgtaatgcaaggaagggacaaagatgaaatggaaacatgttccttttttggtcatgatatcactcctcagcttatccccaaatcacctgtttctggcccagcttattccagaccctgaagatgtagagagggggaacgaccacgggacgccaatccccacctctgataacgatgacaattcgctgggctatacaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtcccgtcttttaccttcacaccaacagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagctgattgtatagcaaattataacaaccaactggataacaaacaaacaaatctgatgctccctgagtcaactgtttatggtgatgtggaccttagtaacaaaatcaatgagatgaaaaccttcaatagcccaaatctgaaggatgggcgttttgtcaatccatcagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctgaagacaaccaagaggcttatgagatctaatgtgaaaatcatcactcaagatgcctcctgtcagatgacacatgacgccagataaaatgttcagtgcaatcagagtgtacaaattgtcgtttttattcctcttattgggatatcattttaaaaactttattgggtttttattgttgttgtttgatccctaaccctacaaagagccttcctattcccctcgctgttggagcaaaccattataccttacttccagcaagcaaagtgctttgacttcttgcttcagtcatcagccagcaagagggaacaaaactgttcttttgcattttgccgctgagatatggcattgcactgcttatatgccaagctaatttatagcaagatattgatcaaatatagaaagttgatattcaacctcacaagggctctcaaagtataatctttctatagccaactgctaatgcaaattaaaacatatttcattttaacatgatttcaaaatcagtttttcatactaccctttgctggaagaaactaaaaatatagcaaatgcagaaccacaaacaattcgaatggggtagaaacattgtaaatatttactctttgcaaaccctggtggtattttattttggcttcatttcaatcattgaagtatattcttattggaaatgtacttttggataagtagggctaagccagttggatctctggttgtctagtcattgtcataagtaaacctagtaaaaccttgttctatttttcaatcatcaaaaagtaattataaatacgtattacaaacaagtggatgtttttaatgaccaattgagtaagaacatccctgtcttaactggcctaaatttcttctggtagtgtcagttcaactttcagaagtgccacttaaggaagtttgatttttgtttttgtaatgcactgtttttaatctctctctctttttttttttttttttggttttaaaagcacaatcactaaactttatttgtaaaccattgtaactattaaccttttttgtcttattgaaaaaaaaaatgttgagaagcgtttttaacctgttttgttaatgctctatgtttgtatttggaatatttgaataatgacagatggtgaagtaacatgcatactttattgtgggccatgaaccaaatggttcttacttttcctggacttaaagaaaaaaagaggtttaagtttgttgtggccaatgtcgaaacctacaagatttccttaaaatctctaatagaggcattacttgctttcaattgacaaatgatgccctctgactagtagatttctatgatccttttttgtcattttatgaatatcattgattttataattggtgctatttgaagaaaaaaatgtacatttattcatagatagataagtatcaggtctgaccccagtggaaaacaaagccaaacaaaactgaaccacaaaaaaaaaggctggtgttcaccaaaaccaaacttgttcatttagataatttgaaaaagttccatagaaaaggcgtgcagtactaagggaacaatccatgtgattaatgttttcattatgttcatgtaagaagccccttatttttagccataattttgcatactgaaaatccaataatcagaaaagtaattttgtcacattatttattaaaaatgttctcaaatacataaaaaaaaaaaaaaaaaaaaaaaaaaaaaaaaaaaaaaaaaaaaaaaaaaaaaaaa

SEQ ID NO: 2 - DNA, 7550 bp, Homo sapiens
aattgagctggagaggaggcagcgtgagagcagaaacttcagacgccgctgatccgggaggagctggggtgagccgcggcggccgtctctcccacccgcagcagcatcctctctgcccttctctgccaccccggggagagccgggagctgcctctttacagcttccacgagccaggggtgcaggcagctgcccccaggaagtttgggcttctgcgtagtttaggggtgcctgcgagcgccccagagggcgaggggccgagggcgatgttgggcgccgcgcggggctgggggcgcccagaagacgtgcgagtgtccgcggtcctgctgctgtctccagtaccctccgcatcccccaagtgatgggaacaagggcccgcccaggcagccgctgtcgccgcaccgccccctcgctcgctctctgcgcgcggagtcacccagtcacactcccggcaccccgagcccttcctccggagctgctgcttctactttggctgctatcgccgccgccgcgggtggcccgctgctgactgggctcgccgggagacggagaagcactttttggccctccctcagcagctctcacaccccaactttgccgccgccgccgcgcctgccctcgcagcggcgctcggccgcacattgtgggggcgcacgccgggaggctccgcaagaccgtggaggcaggaaacggcactactgcgcttctgcctcggctctttgttgttcgctttggatggttcttgaaagtgtctgagcctcctcggaaatcctggggccggagaagacaaaccttggaattcttcctctgcaaaagtctctgagatactgacaagcgtccggaaaggtcgacgagtaattgccctgaaaactcttggctaattgacccacgttgcttatattaagcctttgtgtgtggtgtgtggcttcatacatttggggaccctatttccactccctcctcttggcatgagactgtatacaggatccacccgaggacaatgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagttgggtctgaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagctgattgtatagcaaattataacaaccaactggataacaaacaaacaaatctgatgctccctgagtcaactgtttatggtgatgtggaccttagtaacaaaatcaatgagatgaaaaccttcaatagcccaaatctgaaggatgggcgttttgtcaatccatcagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctgaagacaaccaagaggcttatgagatctaatgtgaaaatcatcactcaagatgcctcctgtcagatgacacatgacgccagataaaatgttcagtgcaatcagagtgtacaaattgtcgtttttattcctcttattgggatatcattttaaaaactttattgggtttttattgttgttgtttgatccctaaccctacaaagagccttcctattcccctcgctgttggagcaaaccattataccttacttccagcaagcaaagtgctttgacttcttgcttcagtcatcagccagcaagagggaacaaaactgttcttttgcattttgccgctgagatatggcattgcactgcttatatgccaagctaatttatagcaagatattgatcaaatatagaaagttgatattcaacctcacaagggctctcaaagtataatctttctatagccaactgctaatgcaaattaaaacatatttcattttaacatgatttcaaaatcagtttttcatactaccctttgctggaagaaactaaaaatatagcaaatgcagaaccacaaacaattcgaatggggtagaaacattgtaaatatttactctttgcaaaccctggtggtattttattttggcttcatttcaatcattgaagtatattcttattggaaatgtacttttggataagtagggctaagccagttggatctctggttgtctagtcattgtcataagtaaacctagtaaaaccttgttctatttttcaatcatcaaaaagtaattataaatacgtattacaaacaagtggatgtttttaatgaccaattgagtaagaacatccctgtcttaactggcctaaatttcttctggtagtgtcagttcaactttcagaagtgccacttaaggaagtttgatttttgtttttgtaatgcactgtttttaatctctctctctttttttttttttttttggttttaaaagcacaatcactaaactttatttgtaaaccattgtaactattaaccttttttgtcttattgaaaaaaaaaatgttgagaagcgtttttaacctgttttgttaatgctctatgtttgtatttggaatatttgaataatgacagatggtgaagtaacatgcatactttattgtgggccatgaaccaaatggttcttacttttcctggacttaaagaaaaaaagaggtttaagtttgttgtggccaatgtcgaaacctacaagatttccttaaaatctctaatagaggcattacttgctttcaattgacaaatgatgccctctgactagtagatttctatgatccttttttgtcattttatgaatatcattgattttataattggtgctatttgaagaaaaaaatgtacatttattcatagatagataagtatcaggtctgaccccagtggaaaacaaagccaaacaaaactgaaccacaaaaaaaaaggctggtgttcaccaaaaccaaacttgttcatttagataatttgaaaaagttccatagaaaaggcgtgcagtactaagggaacaatccatgtgattaatgttttcattatgttcatgtaagaagccccttatttttagccataattttgcatactgaaaatccaataatcagaaaagtaattttgtcacattatttattaaaaatgttctcaaatacataaaaaaaaaaaaaaaaaaaaaaaaaaaaaaaaaaaaaaaaaaaaaaaaaaaaaaaa

SEQ ID NO: 3 - DNA, 7385 bp, Homo sapiens
aattgagctggagaggaggcagcgtgagagcagaaacttcagacgccgctgatccgggaggagctggggtgagccgcggcggccgtctctcccacccgcagcagcatcctctctgcccttctctgccaccccggggagagccgggagctgcctctttacagcttccacgagccaggggtgcaggcagctgcccccaggaagtttgggcttctgcgtagtttaggggtgcctgcgagcgccccagagggcgaggggccgagggcgatgttgggcgccgcgcggggctgggggcgcccagaagacgtgcgagtgtccgcggtcctgctgctgtctccagtaccctccgcatcccccaagtgatgggaacaagggcccgcccaggcagccgctgtcgccgcaccgccccctcgctcgctctctgcgcgcggagtcacccagtcacactcccggcaccccgagcccttcctccggagctgctgcttctactttggctgctatcgccgccgccgcgggtggcccgctgctgactgggctcgccgggagacggagaagcactttttggccctccctcagcagctctcacaccccaactttgccgccgccgccgcgcctgccctcgcagcggcgctcggccgcacattgtgggggcgcacgccgggaggctccgcaagaccgtggaggcaggaaacggcactactgcgcttctgcctcggctctttgttgttcgctttggatggttcttgaaagtgtctgagcctcctcggaaatcctggggccggagaagacaaaccttggaattcttcctctgcaaaagtctctgagatactgacaagcgtccggaaaggtcgacgagtaattgccctgaaaactcttggctaattgacccacgttgcttatattaagcctttgtgtgtggtgtgtggcttcatacatttggggaccctatttccactccctcctcttggcatgagactgtatacaggatccacccgaggacaatgattgcggagcccgctcacttttacctgtttggattaatatgtctctgttcaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagttgggtctgaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctgaagacaaccaagaggcttatgagatctaatgtgaaaatcatcactcaagatgcctcctgtcagatgacacatgacgccagataaaatgttcagtgcaatcagagtgtacaaattgtcgtttttattcctcttattgggatatcattttaaaaactttattgggtttttattgttgttgtttgatccctaaccctacaaagagccttcctattcccctcgctgttggagcaaaccattataccttacttccagcaagcaaagtgctttgacttcttgcttcagtcatcagccagcaagagggaacaaaactgttcttttgcattttgccgctgagatatggcattgcactgcttatatgccaagctaatttatagcaagatattgatcaaatatagaaagttgatattcaacctcacaagggctctcaaagtataatctttctatagccaactgctaatgcaaattaaaacatatttcattttaacatgatttcaaaatcagtttttcatactaccctttgctggaagaaactaaaaatatagcaaatgcagaaccacaaacaattcgaatggggtagaaacattgtaaatatttactctttgcaaaccctggtggtattttattttggcttcatttcaatcattgaagtatattcttattggaaatgtacttttggataagtagggctaagccagttggatctctggttgtctagtcattgtcataagtaaacctagtaaaaccttgttctatttttcaatcatcaaaaagtaattataaatacgtattacaaacaagtggatgtttttaatgaccaattgagtaagaacatccctgtcttaactggcctaaatttcttctggtagtgtcagttcaactttcagaagtgccacttaaggaagtttgatttttgtttttgtaatgcactgtttttaatctctctctctttttttttttttttttggttttaaaagcacaatcactaaactttatttgtaaaccattgtaactattaaccttttttgtcttattgaaaaaaaaaatgttgagaagcgtttttaacctgttttgttaatgctctatgtttgtatttggaatatttgaataatgacagatggtgaagtaacatgcatactttattgtgggccatgaaccaaatggttcttacttttcctggacttaaagaaaaaaagaggtttaagtttgttgtggccaatgtcgaaacctacaagatttccttaaaatctctaatagaggcattacttgctttcaattgacaaatgatgccctctgactagtagatttctatgatccttttttgtcattttatgaatatcattgattttataattggtgctatttgaagaaaaaaatgtacatttattcatagatagataagtatcaggtctgaccccagtggaaaacaaagccaaacaaaactgaaccacaaaaaaaaaggctggtgttcaccaaaaccaaacttgttcatttagataatttgaaaaagttccatagaaaaggcgtgcagtactaagggaacaatccatgtgattaatgttttcattatgttcatgtaagaagccccttatttttagccataattttgcatactgaaaatccaataatcagaaaagtaattttgtcacattatttattaaaaatgttctcaaatacataaaaaaaaaaaaaaaaaaaaaaaaaaaaaaaaaaaaaaaaaaaaaaaaaaaaaaaa

SEQ ID NO: 4 - protein, 1651 aa, Homo sapiens
MKWKHVPFLVMISLLSLSPNHLFLAQLIPDPEDVERGNDHGTPIPTSDNDDNSLGYTGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVPSFTFTPTVTYQRGGEAVSSGGRPGLLNISEPAAQPWLADTWPNTGNNHNDCSISCCTAGNGNSDSNLTTYSRPADCIANYNNQLDNKQTNLMLPESTVYGDVDLSNKINEMKTFNSPNLKDGRFVNPSGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 5 - protein, 1606 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQVGSEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVTYQRGGEAVSSGGRPGLLNISEPAAQPWLADTWPNTGNNHNDCSISCCTAGNGNSDSNLTTYSRPADCIANYNNQLDNKQTNLMLPESTVYGDVDLSNKINEMKTFNSPNLKDGRFVNPSGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 6 - protein, 1551 aa, Homo sapiens
MIAEPAHFYLFGLICLCSGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQVGSEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVTYQRGGEAVSSGGRPGLLNISEPAAQPWLADTWPNTGNNHNDCSISCCTAGNGNSDSNLTTYSRPGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 7 - DNA, 1847 bp, Homo sapiens
ggtaccatagagttgctctgaaaacagaagatagagggagtctcggagctcgccatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 8 - DNA, 2020 bp, Homo sapiens
gcagattcacagggcctctgagcattatcccccatactcctccccatcattctccacccagctgttggagccatctgtctgatcaccttggactccatagtacactggggcaaagcacagccccagtttctggaggcagatgggtaaccaggaaaaggcatgaatgagggggccccaggagacagtgacttagagactgaggcaagagtgccgtggtcaatcatgggtcattgtcttcgaactggacaggccagaatgtctgccacacccacacctgcaggtgaaggagccagaagggatgaactttttgggattctccaaatactccatcagtgtatcctgtcttcaggtgatgcttttgttcttactggcgtctgttgttcctggaggcagaatggcaagccaccatattcacaaaaggaagataaggaagtacaaactggatacatgaatgctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 9 - DNA, 1996 bp, Homo sapiens
gcagattcacagggcctctgagcattatcccccatactcctccccatcattctccacccagctgttggagccatctgtctgatcaccttggactccatagtacactggggcaaagcacagccccagtttctggaggcagatgggtaaccaggaaaaggcatgaatgagggggccccaggagacagtgacttagagactgaggcaagagtgccgtggtcaatcatgggtcattgtcttcgaactggacaggccagaatgtctgccacacccacacctgcaggtgaaggagccagaagctcttcaacctgtagctccctgagcaggctgttctggtctcaacttgagcacataaactgggatggagccacagccaagaactttattaatttaagggagttcttctcttttctgctccctgcattgagaaaagctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 10 - DNA, 1687 bp, Homo sapiens
tgtggctcgggcggcggcggcgcggcggcggcagagggggctccggggtcggaccatccgctctccctgcgctctccgcaccgcgcttaaatgatgtattttgtgatcgcagcgatgaaagctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 11 - protein, 523 aa, Homo sapiens
MESAPAAPDPAASEPGSSGADAAAGSRETPLNQESARKSEPPAPVRRQSYSSTSRGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 12 - protein, 556 aa, Homo sapiens
MNEGAPGDSDLETEARVPWSIMGHCLRTGQARMSATPTPAGEGARRDELFGILQILHQCILSSGDAFVLTGVCCSWRQNGKPPYSQKEDKEVQTGYMNAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 13 - protein, 548 aa, Homo sapiens
MNEGAPGDSDLETEARVPWSIMGHCLRTGQARMSATPTPAGEGARSSSTCSSLSRLFWSQLEHINWDGATAKNFINLREFFSFLLPALRKAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 14 - protein, 468 aa, Homo sapiens
MMYFVIAAMKAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 15 - DNA, 221 bp, Homo sapiens
tagactcatataaccataacacaacccaagaatattaatatcagagagtatttataagtgaaaaagatgtcaattttcctaatgagtttgaaaatattgtatggtataatkctgagacagcaattcagatttttaaaaatcataccatagacgagtactttggtttttatgatttctattctttttattggtcacagttgttttatcacacactggaaatt

SEQ ID NO: 16 - DNA, 221 bp, Homo sapiens
aatttccagtgtgtgataaaacaactgtgaccaataaaaagaatagaaatcataaaaaccaaagtactcgtctatggtatgatttttaaaaatctgaattgctgtctcagmattataccatacaatattttcaaactcattaggaaaattgacatctttttcacttataaatactctctgatattaatattcttgggttgtgttatggttatatgagtcta

SEQ ID NO: 17 - DNA, 221 bp, Homo sapiens
gtgaaaaagtcattgaggtggtgcttcgtgaactagttaagaaaataaaaattctgtagggcagaggtaggcaaacattggctagactttgaggaccatccattctctgtyactacatctcaaaaaccatagaacagcaacattttgaaaataatacagccatagtcaatagataaacaaatgagtgtgatagttttccaataaaaaatgacttataaaaa

SEQ ID NO: 18 - DNA, 221 bp, Homo sapiens
tttttataagtcattttttattggaaaactatcacactcatttgtttatctattgactatggctgtattattttcaaaatgttgctgttctatggtttttgagatgtagtracagagaatggatggtcctcaaagtctagccaatgtttgcctacctctgccctacagaatttttattttcttaactagttcacgaagcaccacctcaatgactttttcac

SEQ ID NO: 19 - DNA, 221 bp, Homo sapiens
tgtagtcaaggcggacaccagaaagattgttagtaaatagggtaggaaggctaggccaatgttatgcagtgtttaaatagtaatggttaagccaatgctttaaaaataagygattaactgttttcaagtgatatacgaagatattttgtgaattcttctgcaggctcccgtcttcgtcaggaagattttccacctcgcattgttgaacacccttcagacct

SEQ ID NO: 20 - DNA, 221 bp, Homo sapiens
aggtctgaagggtgttcaacaatgcgaggtggaaaatcttcctgacgaagacgggagcctgcagaagaattcacaaaatatcttcgtatatcacttgaaaacagttaatcrcttatttttaaagcattggcttaaccattactatttaaacactgcataacattggcctagccttcctaccctatttactaacaatctttctggtgtccgccttgactaca

SEQ ID NO: 21 - DNA, 221 bp, Homo sapiens
aatacaatgtctttgaaaaagaaacgatgtccaattttactgttctttagtccttcttagaaactacctattatttgccatttgaaattgttcctacgttacagaactgtkaaaaatktatgtgttagaactcagttagttttggacagcataatgatgtagaacagtgtgtctgaggaaatatggtgatgaatatatcactgctataacttgtccaaaat

SEQ ID NO: 22 - DNA, 221 bp, Homo sapiens
attttggacaagttatagcagtgatatattcatcaccatatttcctcagacacactgttctacatcattatgctgtccaaaactaactgagttctaacacatamatttttmacagttctgtaacgtaggaacaatttcaaatggcaaataataggtagtttctaagaaggactaaagaacagtaaaattggacatcgtttctttttcaaagacattgtatt

SEQ ID NO: 23 - DNA, 221 bp, Homo sapiens
agtaaaatatgtgattccatatttgtaaaatrttctaaatgttgaaattcttttgatagacagcaaaggtactttaagaacaaaagcatgtttccttagattccataaaarttcaatgagtagttcataatacttaagtgtttattttaaatgtgttcattttagtgtctgtgtttgaayttgctgaatgtatrcattaagctacaattttatggaaaaca

SEQ ID NO: 24 - DNA, 221 bp, Homo sapiens
tgttttccataaaattgtagcttaatgyatacattcagcaarttcaaacacagacactaaaatgaacacatttaaaataaacacttaagtattatgaactactcattgaayttttatggaatctaaggaaacatgcttttgttcttaaagtacctttgctgtctatcaaaagaatttcaacatttagaayattttacaaatatggaatcacatattttact

SEQ ID NO: 25 - DNA, 221 bp, Homo sapiens
cagaattactccatggctaatggttggctgagggaattgactaggctgatatggtttgttctgctgaaaaagatctcccatcctgcagcaggtagccctagctccttgggrttccaaagaacggtaacagagcaagcccctaagcacaaccttttccagcttcttatatcaagttttccaatatttccttggcaaaactaagtcttatggccaactcaaaa

SEQ ID NO: 26 - DNA, 221 bp, Homo sapiens
ttttgagttggccataagacttagttttgccaaggaaatattggaaaacttgatataagaagctggaaaaggttgtgcttaggggcttgctctgttaccgttctttggaaycccaaggagctagggctacctgctgcaggatgggagatctttttcagcagaacaaaccatatcagcctagtcaattccctcagccaaccattagccatggagtaattctg

SEQ ID NO: 27 - DNA, 221 bp, Homo sapiens
ctataggaaattgaggtcctagaaggctaactgactaattcaaaactacataggataaaactgtagaaacagtgttagtcaccgtacctgcaatagatatttcacttaatmcccacataaccctttcaaagtaggctttattagatgtctacaacacatgaagagaatgaagctcagagagtttaaggaaaatagacatgactattcagccaaaaaggggc

SEQ ID NO: 28 - DNA, 221 bp, Homo sapiens
gcccctttttggctgaatagtcatgtctattttccttaaactctctgagcttcattctcttcatgtgttgtagacatctaataaagcctactttgaaagggttatgtgggkattaagtgaaatatctattgcaggtacggtgactaacactgtttctacagttttatcctatgtagttttgaattagtcagttagccttctaggacctcaatttcctatag

SEQ ID NO: 29 - DNA, 221 bp, Homo sapiens
acttgcattttcttaaacactcaggatgtttcattcctctcggcttttgtgtgtgtgtgtgtgtgtgtgtttgtccagaattctgccccaaatggttctcactttcttatytttttagcgatgtttgaaaacacaaaacaagtgtcacttcttctgtgaagaccttcatgttaagaaaataggtttaagtattcctccctttctgatcatttaataatgcc

SEQ ID NO: 30 - DNA, 221 bp, Homo sapiens
ggcattattaaatgatcagaaagggaggaatacttaaacctattttcttaacatgaaggtcttcacagaagaagtgacacttgttttgtgttttcaaacatcgctaaaaarataagaaagtgagaaccatttggggcagaattctggacaaacacacacacacacacacacacaaaagccgagaggaatgaaacatcctgagtgtttaagaaaatgcaagt

SEQ ID NO: 31 - DNA, 221 bp, Homo sapiens
gaggtaatgtctaagtggtcattcattcacacatgtaattcacatattccattctgtatcattagaaaatggattttaatgcaagaaggggttgttacgattcagagcacwggctctcaaactttgctacgtgttagaatcaccaagggaactttaacaatttcaataaccaggtagcatccagacaaattaaaacaatctccaaaaatgcccagggttag

SEQ ID NO: 32 - DNA, 221 bp, Homo sapiens
ctaaccctgggcatttttggagattgttttaatttgtctggatgctacctggttattgaaattgttaaagttcccttggtgattctaacacgtagcaaagtttgagagccwgtgctctgaatcgtaacaaccccttcttgcattaaaatccattttctaatgatacagaatggaatatgtgaattacatgtgtgaatgaatgaccacttagacattacctc

SEQ ID NO: 33 - DNA, 221 bp, Homo sapiens
aactaaacaattatatgccaataaagcccacatattataaatgtttgtctacagaataagagaataatgtgtaattaacttgaccagcctccaacaaaacccatgctaaayagaagaaggtcacttattttgatgagcagactctaattgcttcatttatatttttgattttttctcagagataattagaaaacggatgccrgatcctgcattctgtttta

SEQ ID NO: 34 - DNA, 221 bp, Homo sapiens
taaaacagaatgcaggatcyggcatccgttttctaattatctctgagaaaaaatcaaaaatataaatgaagcaattagagtctgctcatcaaaataagtgaccttcttctrtttagcatgggttttgttggaggctggtcaagttaattacacattattctcttattctgtagacaaacatttataatatgtgggctttattggcatataattgtttagtt

SEQ ID NO: 35 - DNA, 221 bp, Homo sapiens
tttaagctctatggccaacctgttgarctaggtgtcctatctacagactgagtgtatgaatgggtggaaacaagatgatgaaaattacagagagaactgaattagacaacyagttatttgaaaatgcatatccttcgagaatagtagaaagtaagtagagaaatttactaatatatccatccaaaggaatccaaattttcttccttgagtgagtagagtat

SEQ ID NO: 36 - DNA, 221 bp, Homo sapiens
atactctactcactcaaggaagaaaatttggattcctttggatggatatattagtaaatttctctacttactttctactattctcgaaggatatgcattttcaaataactrgttgtctaattcagttctctctgtaattttcatcatcttgtttccacccattcatacactcagtctgtagataggacacctagytcaacaggttggccatagagcttaaa

SEQ ID NO: 37 - DNA, 221 bp, Homo sapiens
cttacactaacactctgcagactctagaaaatgagattcgtttttttcctttgacacactgtttgtggaagtgcccctgagtcatatcattatatctaagatgaccaattrctttttctgaggatagaaattcaagatgaagttatttgaaggactaaggagagtaatgatgaatttttcatatgytcttattctattttctcgctgtaaaaaatgtataa

SEQ ID NO: 38 - DNA, 221 bp, Homo sapiens
ttatacattttttacagcgagaaaatagaataagarcatatgaaaaattcatcattactctccttagtccttcaaataacttcatcttgaatttctatcctcagaaaaagyaattggtcatcttagatataatgatatgactcaggggcacttccacaaacagtgtgtcaaaggaaaaaaacgaatctcattttctagagtctgcagagtgttagtgtaag

SEQ ID NO: 39 - DNA, 221 bp, Homo sapiens
tcacaaggccagcctagatttaagggatgggaaaatggacttcggctcttgatgggagcagtctcagtcgcattggrtaggacacaacatagggaagtcattaattcggaygatcagtggaatcaatctaccatattttcaaataatatggtagattatgayattaatctaccatattaaawtaaaattttgctaacctaagaaaaggttagcaaaatgca

SEQ ID NO: 40 - DNA, 221 bp, Homo sapiens
tgcattttgctaaccttttcttaggttagcaaaattttawtttaatatggtagattaatrtcataatctaccatattatttgaaaatatggtagattgattccactgatcrtccgaattaatgacttccctatgttgtgtcctayccaatgcgactgagactgctcccatcaagagccgaagtccattttcccatcccttaaatctaggctggccttgtga

SEQ ID NO: 41 - DNA, 221 bp, Homo sapiens
tcccccatcagaattactacaatagaatatatgggggtggggcacttgagtccacatattaacagaatctattccaggtgtaactaggaacagggagtttatcacaacaaytgctctccaattcagtcagatcaatatggcacttaatttagcatttgggggaggagccatttgcaaagctttttagatcttattttgtgtcttcccagattaccgtgctt

SEQ ID NO: 42 - DNA, 221 bp, Homo sapiens
aagcacggtaatctgggaagacacaaaataagatctaaaaagctttgcaaatggctcctcccccaaatgctaaattaagtgccatattgatctgactgaattggagagcaraagcacggtaatctgggaagacacaaaataagatctaaaaagctttgcaaatggctcctcccccaaatgctaaattaagtgccatattgatctgactgaattggagagca